SEQUENCE LISTING (63 sequences)

SEQ ID NO: 1 - protein, 21 aa, Homo sapiens
EEQYNSTYRVVSVLTVLHQDW

SEQ ID NO: 2 - protein, 26 aa, Homo sapiens
PAVLQSSGLYSLSSVVTVPSSSLGTQ

SEQ ID NO: 3 - protein, 15 aa, Homo sapiens
PGLVRPSQTLSLTCT

SEQ ID NO: 4 - protein, 21 aa, Homo sapiens
GGLVQPGGSLRLSCAASGFTF

SEQ ID NO: 5 - protein, 21 aa, Homo sapiens
GGLVQPGRSLRLSCAASGFTF

SEQ ID NO: 6 - protein, 15 aa, Homo sapiens
GASVKVSCKASGYTF

SEQ ID NO: 7 - protein, 15 aa, Homo sapiens
WSWVRQPPGRGLEWI

SEQ ID NO: 8 - protein, 15 aa, Homo sapiens
WSWIRQPPGKGLEWI

SEQ ID NO: 9 - protein, 15 aa, Homo sapiens
MHWVRQAPGKGLEWV

SEQ ID NO: 10 - protein, 15 aa, Homo sapiens
MHWVRQAPGQGLEWM

SEQ ID NO: 11 - protein, 21 aa, Homo sapiens
VDTSKNQFSLRLSSVTAADTA

SEQ ID NO: 12 - protein, 21 aa, Homo sapiens
NTLYLQMNSLRAEDTAVYYCA

SEQ ID NO: 13 - protein, 14 aa, Homo sapiens
FQHWGQGTLVTVSS

SEQ ID NO: 14 - protein, 14 aa, Homo sapiens
FDLWGRGTLVTVSS

SEQ ID NO: 15 - protein, 14 aa, Homo sapiens
FDIWGQGTMVTVSS

SEQ ID NO: 16 - protein, 14 aa, Homo sapiens
FDYWGQGTLVTVSS

SEQ ID NO: 17 - protein, 14 aa, Homo sapiens
FDPWGQGTLVTVSS

SEQ ID NO: 18 - protein, 14 aa, Homo sapiens
MDVWGQGTLVTVSS

SEQ ID NO: 19 - protein, 14 aa, Homo sapiens
MDVWGQGTTVTVSS

SEQ ID NO: 20 - protein, 24 aa, Homo sapiens
LNNFYPREAKVQWKVDNALQSGNS

SEQ ID NO: 21 - protein, 14 aa, Homo sapiens
KVYACEVTHQGLSS

SEQ ID NO: 22 - protein, 13 aa, Homo sapiens
DIQMTQSPSSLSA

SEQ ID NO: 23 - protein, 13 aa, Homo sapiens
EIVLTQSFGTLSL

SEQ ID NO: 24 - protein, 15 aa, Homo sapiens
GDRVTITCRASQGIS

SEQ ID NO: 25 - protein, 14 aa, Homo sapiens
LAWYQQKPGKAPKL

SEQ ID NO: 26 - protein, 14 aa, Homo sapiens
LAWYQQKPGQAPRL

SEQ ID NO: 27 - protein, 15 aa, Homo sapiens
LLIYGASSRATGIPD

SEQ ID NO: 28 - protein, 15 aa, Homo sapiens
GTDFTLTISSLQPED

SEQ ID NO: 29 - protein, 13 aa, Homo sapiens
SYELTQPPSVSVS

SEQ ID NO: 30 - protein, 15 aa, Homo sapiens
GQSITISCTGTSSDV

SEQ ID NO: 31 - protein, 14 aa, Homo sapiens
VSWYQQHPGKAPKL

SEQ ID NO: 32 - protein, 14 aa, Homo sapiens
VHWYQQKPGQAPVL

SEQ ID NO: 33 - protein, 14 aa, Homo sapiens
VSWYQQLPGTAPKL

SEQ ID NO: 34 - protein, 15 aa, Homo sapiens
LMIYEVSNRPSGVPD

SEQ ID NO: 35 - protein, 19 aa, Homo sapiens
LKKYLYEIARRHPYFYAPE

SEQ ID NO: 36 - protein, 22 aa, Homo sapiens
APELLFFAKRYKAAFTECCQAA

SEQ ID NO: 37 - protein, 21 aa, Homo sapiens
HPDYSVVLLLRLAKTYETTLE

SEQ ID NO: 38 - protein, 15 aa, Homo sapiens
HPDYSVYLLLRLAKT

SEQ ID NO: 39 - protein, 14 aa, Homo sapiens
LLLRLAKTYETTLE

SEQ ID NO: 40 - protein, 25 aa, Homo sapiens
LGEYKFQNALLVRYTKKVPQVSTPT

SEQ ID NO: 41 - protein, 22 aa, Homo sapiens
PADVAIQLTFLRLMSTEASQNI

SEQ ID NO: 42 - protein, 19 aa, Homo sapiens
TGNLKKALLLQGSNEIEIR

SEQ ID NO: 43 - protein, 16 aa, Homo sapiens
DGDFYRADQPRSAPSL

SEQ ID NO: 44 - protein, 25 aa, Homo sapiens
SKEMATQLAFMRLLANYASQNITYH

SEQ ID NO: 45 - protein, 19 aa, Homo sapiens
VQHIQLLQKNVRAQLVDMK

SEQ ID NO: 46 - protein, 23 aa, Homo sapiens
GEFWLGNDYLHLLTQRQSVLRVE

SEQ ID NO: 47 - protein, 21 aa, Homo sapiens
QSGLYFIKPLKANQQFLVYCE

SEQ ID NO: 48 - protein, 21 aa, Homo sapiens
TEFWLGNEKIHLISTQSAIPY

SEQ ID NO: 49 - protein, 25 aa, Homo sapiens
NANFKFTDHLKYVMLPVADQDQCIR

SEQ ID NO: 50 - protein, 26 aa, Homo sapiens
GSEVVKRPRRYLYQWLGAPVPYPDPL

SEQ ID NO: 51 - protein, 16 aa, Homo sapiens
PCQWWRPTTTSTRCCT

SEQ ID NO: 52 - protein, 22 aa, Homo sapiens
PGEDFRMATLYSRTQTPRAELK

SEQ ID NO: 53 - protein, 18 aa, Homo sapiens
DGSLWRYRAGLAASLAGP

SEQ ID NO: 54 - protein, 21 aa, Homo sapiens
VTGVVLFRQLAPRAKLDAFFA

SEQ ID NO: 55 - protein, 19 aa, Homo sapiens
KASYLDCIRAIAANEADAV

SEQ ID NO: 56 - protein, 15 aa, Homo sapiens
LQPLALEGSLQKRGI

SEQ ID NO: 57 - protein, 15 aa, Homo sapiens
SHLVEALYLVCGERG

SEQ ID NO: 58 - protein, 12 aa, Homo sapiens
SHLVEALYLVCG

SEQ ID NO: 59 - protein, 15 aa, Homo sapiens
LCGSHLVEALYLVCG

SEQ ID NO: 60 - protein, 15 aa, Homo sapiens
AAAFVNQHLCGSHLV

SEQ ID NO: 61 - protein, 20 aa, Homo sapiens
GAGSLQPLALEGSLQKRGIV

SEQ ID NO: 62 - protein, 18 aa, Homo sapiens
GSLQPLALEGSLQKRGIV

SEQ ID NO: 63 - protein, 18 aa, Homo sapiens
RGFFYTPKTRREAEDLQV

(19) United States
(12) Patent Application Publication
(10) Pub. No.: US 2023/0158165 A1
(43) Pub. Date: May 25, 2023

(54) TREGITOPE CONSTRUCTS USEFUL IN THE PREVENTION AND TREATMENT OF TYPE 1 DIABETES

(71) Applicant: EPIVAX, INC., Providence, RI (US)

(72) Inventors: Anne De Groot, Providence, RI (US); William Martin, Providence, RI (US)

(21) Appl. No.: 17/906,817

(22) PCT Filed: Mar. 26, 2021
(86) PCT No.: PCT/US2021/024377

§ 371 Date: Sep. 20, 2022

Related U.S. Application Data
(60) Provisional application No. 63/000,590, filed on Mar. 27, 2020.

(51) Int. Cl. A61K 47/64; A61K 47/54; A61P 37/06; A61P 3/10

(52) U.S. Cl. CPC A61K 47/643; A61K 47/545; A61P 37/06; A61P 3/10

(57) ABSTRACT

The present disclosure generally relates to novel Tregitope-blood component conjugates and modified polypeptides comprising Tregitopes, with said modified peptides being capable of reacting with blood components to form such Tregitope-blood component conjugates. In aspects, the Tregitope-blood component conjugates include a blood component which acts as a carrier protein (e.g., albumin), and further include a modified polypeptide comprising one or more regulatory T cell epitopes (termed “Tregitopes”), the polypeptide having been modified by attaching a reactive moiety to the polypeptide that is capable of forming a bond (e.g., a covalent linkage) with a reactive functionality on the blood component. The present disclosure also relates to methods of using said Tregitope-blood component conjugates and modified polypeptides comprising Tregitopes in the treatment and prevent of autoimmune disorders, such as type 1 diabetes.

### CROSS-REFERENCE TO RELATED APPLICATION

This application depends from and claims priority to U.S. Provisional Application No. 63/000,590 filed Mar. 27, 2020, the entire contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Mar. 24, 2021, is named EPV0025WO_ST25.txt and is 14 KB in size.

### FIELD OF THE INVENTION

The present disclosure generally relates to novel Tregitope-blood component conjugates and modified polypeptides comprising Tregitopes, with said modified peptides being capable of reacting with blood components to form such Tregitope-blood component conjugates. In aspects, the Tregitope-blood component conjugates include a blood component which acts as a carrier protein (e.g., albumin), and further include a modified polypeptide comprising one or more regulatory T cell epitopes (termed “Tregitopes”), the polypeptide having been modified by attaching a reactive moiety to the polypeptide that is capable of forming a bond (e.g., a covalent linkage) with a reactive functionality on the blood component. In aspects, the modified polypeptide further comprises one or more antigen peptides associated with immunogenicity in diabetes (“T1Dgen” peptides; e.g., PPI-derived peptides), which may be optionally separated from one or more Tregitopes by a cleavage site (e.g., a lysosomal cleavage site). The present disclosure also relates to methods of using said Tregitope-blood component conjugates and modified polypeptides comprising Tregitopes in the treatment and prevent of autoimmune disorders, such as type 1 diabetes.

### BACKGROUND

Type 1 diabetes (T1D) is an autoimmune disease affecting 1,250,000 Americans that is caused by the destruction of insulin-producing pancreatic islet cells and increasing 2-3% per year worldwide, especially among children. Autoimmune T1D may be induced by environmental factors and accelerated by defects in the regulation of T cell response by antigen-specific regulatory T cells (Tregs). In the absence of effective regulation, CD8+ and CD4+ auto-reactive T cells target pancreatic islet cell antigens presented by human leukocyte antigen (HLA) molecules. Gradual destruction of pancreatic islet cells by T cells leads to glucose intolerance and life-long dependence on insulin replacement therapies.

Reducing islet cell destruction and preserving islet cell function is believed to be critically important to developing a cure for T1D. Tregs protect islet cell damage by T effector cells (Teff). As a result, preservation, expansion, and/or activation of islet antigen-specific Tregs to restore tolerance is a key area of T1D research. Both adoptive Treg therapy and monoclonal antibodies that drive Treg expansion are being explored, but neither approach is antigen-specific, nor have they met critical efficacy endpoints. We propose that Tregitope will improve outcomes of antigen-specific therapies by adding the power of natural Treg induction to the antigen-specificity of selected PPI peptides. As such, there is need in the art for compositions containing such Tregitopes and T1D-related antigens (e.g., peptides), and for methods related to their preparation and use in the treatment of T1D.

### SUMMARY

Accordingly, the aim of the present disclosure is to provide novel Tregitope-blood component conjugates and modified polypeptides comprising Tregitopes, with said modified peptides being capable of reacting with blood components to form such Tregitope-blood component conjugates. Conjugation of a polypeptide comprising a Tregitope with a blood component, such as albumin, can be useful as a carrier protein for a Tregitope payload. Tregitope-blood component conjugates can extend the half-life of the modified polypeptides comprising Tregitopes in vivo, protect the modified polypeptides comprising Tregitopes from rapid proteolytic degradation, protect the modified polypeptides comprising Tregitopes from rapid clearance from circulation and/or rapid kidney excretion, allow for wide distribution of Tregitope-blood component conjugates throughout the body of a subject, aid in delivery of modified polypeptides comprising Tregitopes to appropriate immune cells (such as macrophages and APCs), allow the modified polypeptides comprising Tregitopes to be processed by the endocytic pathway of certain immune cells (such as macrophages and APCs), and aid in the presentation of modified polypeptides comprising Tregitopes as an antigen by said immune cells.

The selective engagement and activation of naturally occurring Tregs (in aspects, including natural Tregs and/or adaptive Tregs) through the use of Tregitope-blood component conjugates and modified polypeptides comprising Tregitopes as disclosed herein, is therapeutically valuable as a means of treatment for a disease or condition marked by the presence of an unwanted immune response, for example autoimmune disease such as type 1 diabetes. As such, the present disclosure also relates to methods of using said Tregitope-blood component conjugates and modified polypeptides comprising Tregitopes in the treatment and prevention of autoimmune disorders, such as type 1 diabetes.

In aspects, the Tregitope-blood component conjugates comprise a blood component which acts as a carrier protein (e.g., albumin), and further comprise a modified polypeptide, said modified polypeptide comprising one or more regulatory T cell epitopes (termed “Tregitopes”). The modified polypeptide comprises a reactive moiety that is attached to the polypeptide, with the reactive moiety being capable of forming a bond (e.g., a covalent linkage) with a reactive functionality on the blood component. In aspects, the modified polypeptide further comprises one or more antigen peptides associated with immunogenicity in diabetes (“T1Dgen” peptides; e.g., PPI-derived peptides), which may be optionally separated from one or more Tregitopes by a linker and/or cleavage site (e.g., a lysosomal cleavage site). Tregitope-blood component conjugates may be formed by modifying a polypeptide comprising a Tregitope by attaching a reactive moiety to the polypeptide to create a modified polypeptide, then forming a bond between reactive moiety of the modified polypeptide with a reactive functionality on a blood component, as disclosed in U.S. Pat. Nos. 6,849,714, 6,887,470, 7,256,253, and 7,307,148, herein incorporated by reference in their entireties. In aspects of above-described Tregitope-blood component conjugates and modified polypeptides comprising Tregitopes, the Tregitope-blood component conjugates and modified polypeptides comprising Tregitopes may be isolated, synthetic, or recombinant.

In aspects, the blood components of the Tregitope-blood component conjugates may be either fixed or mobile, as disclosed in U.S. Pat. Nos. 6,849,714, 6,887,470, 7,256,253, and 7,307,148. Fixed blood components are non-mobile blood components and include tissues, membrane receptors, interstitial proteins, fibrin proteins, collagens, platelets, endothelial cells, epithelial cells and their associated membrane and membranous receptors, somatic body cells, skeletal and smooth muscle cells, neuronal components, osteocytes and osteoclasts and all body tissues, especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1 μg/ml. Mobile blood components include serum albumin, transferrin, ferritin and immunoglobulins such as IgM and IgG. The half-life of mobile blood components is at least about 12 hours. In aspects of the Tregitope-blood component conjugates, the blood component is albumin, such as serum albumin, human serum albumin, recombinant albumin, and recombinant human serum albumin. Albumin is a preferred blood component because it contains an Fc neonatal binding domain that will carry the Tregitope-albumin conjugate into the appropriate cells, such as macrophages and APCs. Further, albumin contains a cysteine at amino acid 34 (Cys34) (the location of the amino acid in the amino acid sequence of human serine albumin), containing a free thiol with a pKa of approximately 5, which may serve as a preferred reactive functionality of albumin. Cys34 of albumin is capable of forming a stable thioester bond with maleimidopropionamido (MPA), which is a preferred reactive moiety of a modified Tregitope peptide.

In aspects, reactive functionalities on the blood component of the Tregitope-blood component conjugates or on the blood components that are capable of forming a conjugate with the instantly-disclosed modified polypeptides are groups on blood components, including mobile and fixed proteins, to which reactive groups on modified therapeutic peptides react to form covalent bonds. As disclosed in U.S. Pat. Nos. 6,849,714, 6,887,470, 7,256,253, and 7,307,148, such functionalities usually include hydroxyl groups for bonding to ester reactive groups, thiol groups for bonding to maleimides, imidates and thioester groups; amino groups for bonding to activated carboxyl, phosphoryl or any other acyl groups on reactive groups.

In aspects, the modified polypeptides of the Tregitope-blood component conjugates and the modified polypeptides used to form the Tregitope-blood component conjugates comprise a reactive moiety that is attached to the polypeptide, with the reactive moiety being capable of forming a bond (e.g., a covalent linkage) with a reactive functionality on the blood component. In aspects, the reactive group is capable of reacting with an amino group, a hydroxyl group, or a thiol group on blood component to form a covalent bond therewith. In aspects, the reactive group is placed at a site such that when the modified polypeptide is bonded to the blood component, the modified peptide retains a substantial proportion of the parent compound's activity. In aspects, the reactive moiety may be a succinimidyl or maleimido group. In aspects, the reactive moiety may be attached to an amino acid positioned in the less therapeutically active region of amino acids of the polypeptide to be modified. In aspects, the reactive moiety is attached to the amino terminal amino acid of the modified polypeptide. In aspects, the reactive moiety is attached to the carboxy terminal amino acid of the modified polypeptide. In aspects, the reactive moiety is attached to an amino acid positioned between the amino terminal amino acid and the carboxy terminal amino acid of the modified polypeptide. In aspects, the reactive group may be attached to the polypeptide (to be modified) either via a linking group, or optionally without using a linking group. Further, one or more additional amino acids (e.g., one or more lysines) may be added to the polypeptide to facilitate the attachment of the reactive group. Linking groups are chemical moieties that link or connect reactive groups of blood components to polypeptides comprising one or more Tregitopes. Linking groups may comprise one or more alkyl groups, alkoxy group, alkenyl group, alkynyl group or amino group substituted by alkyl groups, cycloalkyl group, polycyclic group, aryl groups, polyaryl groups, substituted aryl groups, heterocyclic groups, and substituted heterocyclic groups. Linking groups may also comprise poly ethoxy aminoacids such as AEA ((2-amino)ethoxy acetic acid) or a preferred linking group AEEA ([2-(2-amino)ethoxy)]ethoxy acetic acid). In aspects, linking groups may comprise a polyethylene glycol linker (e.g. but not limited to, PEG2 or PEG12).

As should be understood, modified polypeptides may be administered in vivo such that conjugation with blood components occurs in vivo, or they may be first conjugated to blood components in vitro and the resulting peptidase stabilized polypeptide administered in vivo. Further, as disclosed in in U.S. Pat. Nos. 6,849,714, 6,887,470, 7,256,253, and 7,307,148, a peptidase-stabilized polypeptide is a modified polypeptide that has been conjugated to a blood component via a covalent bond formed between the reactive group of the modified peptide and the functionalities of the blood component, with or without a linking group. Such reaction is preferably established by covalent bonding of a polypeptide modified with a maleimide link (e.g. prepared from GMBS, MPA, or other maleimides) to a thiol group on a mobile blood protein such as serum albumin or IgG. Peptidase-stabilized polypeptides are more stable in the presence of peptidases in vivo than a non-stabilized peptide. A peptidase stabilized therapeutic peptide generally has an increased half-life of at least 10-50% as compared to a non-stabilized peptide of identical sequence. Peptidase stability is determined by comparing the half-life of the unmodified therapeutic peptide in serum or blood to the half-life of a modified counterpart therapeutic peptide in serum or blood. Half-life is determined by sampling the serum or blood after administration of the modified and non-modified peptides and determining the activity of the peptide. In addition to determining the activity, the length of the therapeutic peptide may also be measured.

In aspects, the modified polypeptides of the Tregitope-blood component conjugates and the modified polypeptides used to form the Tregitope-blood component conjugates comprise one or more Tregitopes (which may be termed herein as “T_{reg} activating regulatory T-cell epitope”, “Tregitope”, or “T-cell epitope polypeptide”). In aspects, the one or more Tregitopes of the modified polypeptides have a sequence comprising, consisting of, or consisting essentially of one or more of SEQ ID NOS: 1-55 (and fragments and variants thereof). The phrase “consisting essentially of” is intended to mean that a Tregitope of the modified polypeptides according to the present disclosure, in addition to the sequence according to any of SEQ ID NOS: 1-55 (or a fragment or variant thereof), contains additional amino acids or residues that may be present at either terminus of the peptide and/or on a side chain that are not necessarily forming part of the peptide that functions as an MHC ligand and provided they do not substantially impair the activity of the peptide to function as a Tregitope. In aspects, the Tregitope of the modified polypeptides according to the present disclosure comprises, consists of, or consists essentially of one or more of SEQ ID NOS: 1 and 28. In aspects, the Tregitope of the modified polypeptides according to the present disclosure comprises, consists of, or consists essentially of the amino acid sequence of SEQ ID NO: 1. In aspects, the Tregitope of the modified polypeptides according to the present disclosure comprises, consists of, or consists essentially of the amino acid sequence of SEQ ID NO: 28. In aspects, the one or more Tregitopes of the modified polypeptide may optionally have one or more linkers, which may optionally be cleavage sensitive sites, adjacent to their N-terminal and/or C-terminal end. In such a modified polypeptide, two or more of the Tregitopes may have a cleavage sensitive site between them. Alternatively, two or more of the Tregitopes may be connected directly to one another or through a linker that is not a cleavage-sensitive site. The polypeptides of the present disclosure may be isolated, synthetic, and/or recombinant, and may comprise post-transcriptional modifications such as glycosylation, added chemical groups, etc. In aspects, the peptides or polypeptides can be either in neutral (uncharged) or salt forms, and may be either free of or include modifications such as glycosylation, side chain oxidation, or phosphorylation. In certain aspects, the Tregitopes can be capped with an N-terminal acetyl and/or C-terminal amino group. In aspects, the one or more Tregitopes included in the modified polypeptide can be capped with an N-terminal acetyl and/or C-terminal amino group.

In aspects, the one or more Tregitopes of the modified polypeptides of the Tregitope-blood component conjugates and the modified polypeptides used to form the Tregitope-blood component conjugates have a sequence comprising, consisting of, or consisting essentially of one or more of SEQ ID NOS: 1-55 and (and/or fragments or variants thereof), and optionally 1 to 12 additional amino acids distributed in any ratio on the N terminus and/or C-terminus of the polypeptide of SEQ ID NOS: 1-55. In aspects, the one or more Tregitopes have a core amino acid sequence comprising, consisting of, or consisting essentially of one or more peptides or polypeptides having an amino acid sequence of SEQ ID NOS: 1-55, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, one or more Tregitopes have a core sequence comprising, consisting of, or consisting essentially of one or more peptides or polypeptides having an amino acid sequence of SEQ ID NOS: 1-55 (and/or fragments and variants thereof), optionally with extensions of 1 to 12 amino acids on the C-terminal and/or the N-terminal of the core sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the Tregitope with the flanking amino acids is still able to bind to the same HLA molecule (i.e., retain MHC binding propensity) as said polypeptide core sequence without said flanking amino acids. In aspects, said Tregitope with the flanking amino acids is still able to bind to the same HLA molecule (i.e., retain MHC binding propensity) and/or retain the same TCR specificity as said Tregitope core sequence without said flanking amino acids. In aspects, said polypeptide with the flanking amino acids is still able to bind to a same HLA molecule (i.e., retain MHC binding propensity) and/or retain the same TCR specificity, and/or retain Tregitope activity, as said polypeptide core sequence without said flanking amino acids. In aspects, said flanking amino acid sequences are those that also flank the peptides or polypeptides included therein in the naturally occurring protein, e.g., in an IgG antibody. In aspects, the extension(s) may serve and be designed to improve the biochemical properties of the peptides or polypeptides (e.g., but not limited to, solubility or stability) or to improve the likelihood for efficient proteasomal processing of the peptide. In aspects, said flanking amino acid sequences as described herein may serve as a MHC stabilizing region. The use of a longer peptide may allow endogenous processing by patient cells and may lead to more effective antigen presentation and induction of T cell responses. In aspects, the one or more Tregitopes of the modified polypeptide may optionally have one or more linkers, which may optionally be cleavage sensitive sites, adjacent to their N-terminal and/or C-terminal end. In such a modified polypeptide, two or more of the Tregitopes may have a cleavage sensitive site between them. Alternatively, two or more of the Tregitopes may be connected directly to one another or through a linker that is not a cleavage sensitive site. The polypeptides may be isolated, synthetic, and/or recombinant. In aspects, the modified polypeptide comprising the one or more Tregitopes and/or the Tregitopes contained therein can be in either neutral (uncharged) or salt forms, and may be either free of or include modifications such as glycosylation, side chain oxidation, or phosphorylation. In certain aspects, the modified polypeptide comprising the one or more Tregitopes peptides or polypeptides can be capped with an N-terminal acetyl and/or C-terminal amino group.

In aspects, the modified polypeptides of the Tregitope-blood component conjugates and the modified polypeptides used to form the Tregitope-blood component conjugates further comprise one or more T1Dgen peptides (e.g., PPI-derived peptides). In aspects, said one or more T1Dgen peptides may be optionally separated from one or more Tregitopes by a cleavage site (e.g., a lysosomal cleavage site). In aspects, said one or more T1Dgen peptides of the modified polypeptides comprise, consist, or consist essentially of an amino acid sequence of SEQ ID NOS: 56-63 (and/or fragments or variants thereof). The phrase “consisting essentially of” is intended to mean that a T1Dgen peptide of the modified polypeptides according to the present disclosure, in addition to the sequence according to any of SEQ ID NOS: 56-63 (or a fragment or variant thereof), contains additional amino acids or residues that may be present at either terminus of the peptide and/or on a side chain that are not necessarily forming part of the peptide that functions as an MHC ligand and provided they do not substantially impair the activity of the peptide to function as a an antigen. In aspects, the one or more T1Dgen peptides comprises, consists of, or consists essentially of the amino acid sequence of SEQ ID NO: 63. In aspects, the one or more T1Dgen peptides of the modified polypeptide may optionally have one or more linkers, which may optionally be cleavage sensitive sites, adjacent to their N-terminal and/or C-terminal end. In such a modified polypeptide, two or more of the T1Dgen peptides may have a cleavage sensitive site between them. Alternatively two or more of the T1Dgen peptides may be connected directly to one another or through a linker that is not a cleavage sensitive site. Further, in aspects, a T1Dgen peptide and a Tregitope of the modified polypeptide may have a cleavage sensitive site between them or may be connected directly to one another or through a linker that is not a cleavage sensitive site.

In aspects, said one or more T1Dgen peptides of the modified polypeptides of the Tregitope-blood component conjugates and the modified polypeptides used to form the Tregitope-blood component conjugates comprise, consist, or consist essentially of an amino acid sequence of SEQ ID NOS: 56-63 (and/or fragments or variants thereof), and optionally 1 to 12 additional amino acids distributed in any ratio on the N-terminus and/or C-terminus of the polypeptide of SEQ ID NOS: 56-63. In aspects, said one or more T1Dgen peptides have a core amino acid sequence comprising, consisting of, or consisting essentially of one or more peptides or polypeptides having an amino acid sequence of SEQ ID NOS 56-63, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, said one or more T1Dgen peptides have a core sequence comprising, consisting of, or consisting essentially of one or more peptides or polypeptides having an amino acid sequence of SEQ ID NOS. 56-63 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the T1Dgen peptide with the flanking amino acids is still able to bind to an HLA molecule (i.e., retain MHC binding). In aspects, said T1Dgen peptide with the flanking amino acids is still able to bind to the same HLA molecule (i.e., retain MHC binding propensity) and/or retain the same TCR specificity as said T1Dgen peptide core sequence without said flanking amino acids. In aspects, said flanking amino acid sequences are those that also flank the said T1Dgen peptide included therein in the naturally occurring protein. For example, for a peptide or polypeptide have a core sequence comprising, consisting of, or consisting essentially of one or more peptides or polypeptides having an amino acid sequence of SEQ ID NOS: 56-63 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 56-63 in the amino acid sequence of preproinsulin. In aspects, the extension(s) may serve and be designed to improve the biochemical properties of the peptides or polypeptides (e.g., but not limited to, solubility or stability) or to improve the likelihood for efficient proteasomal processing of the peptide. In aspects, said flanking amino acid sequences as described herein may serve as a MHC stabilizing region. The use of a longer peptide may allow endogenous processing by patient cells and may lead to more effective antigen presentation and induction of T cell responses. In aspects, the one or more T1Dgen peptides can be in either neutral (uncharged) or salt forms, and may be either free of or include modifications such as glycosylation, side chain oxidation, or phosphorylation.

In aspects, the present disclosure is directed to a nucleic acid (e.g., DNA or RNA, including mRNA), which in aspects may be isolated, synthetic, or recombinant, encoding the instantly-disclosed Tregitope-blood component conjugates and/or the modified polypeptides used to form the Tregitope-blood component conjugates. In aspects, the present disclosure is directed to expression cassettes, plasmids, expression vectors, recombinant viruses, or cells comprising a nucleic acid as described herein. In aspects, the present disclosure is directed to a cell or vaccine comprising such a vector as described. In aspects, the present disclosure is directed to a cell comprising a vector of the present disclosure.

In aspects, the present disclosure is directed to a pharmaceutical composition or formulation comprising a compound or composition comprising the instantly disclosed Tregitope-blood component conjugates and/or the modified polypeptides used to form the Tregitope-blood component conjugates (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein), and a pharmaceutically acceptable carrier, excipient, and/or adjuvant.

In aspects, the present disclosure is directed to a method of stimulating, inducing, and/or expanding regulatory T-cells (in aspects, naturally occurring T_{Regs}, including natural T_{Regs} and/or adaptive T_{Regs}) in a subject in need thereof and/or suppressing an autoimmune response associated with T1D in a subject in need thereof by administering to the subject a therapeutically effect amount of compound or composition comprising the instantly disclosed Tregitope-blood component conjugates and/or the modified polypeptides used to form the Tregitope-blood component conjugates (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein).

In aspects, the present disclosure is directed to a method of treating or preventing a medical condition in a subject in need thereof comprising administering a therapeutically effect amount of the instantly disclosed Tregitope-blood component conjugates and/or the modified polypeptides used to form the Tregitope-blood component conjugates (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein). In aspects, the medical condition is selected from the group consisting of: an allergy, an autoimmune disease, a transplant related disorder, graft versus host disease, a blood clotting disorder, an enzyme or protein deficiency disorder, a hemostatic disorder, cancer, infertility; and a viral, bacterial or parasitic infection. In another embodiment, the medical condition is hemophilia A, B, or C. In aspects, the present disclosure is directed to a method of treating or preventing Type 1 Diabetes in a subject in need thereof comprising administering to the subject a therapeutically effect amount of the instantly-disclosed Tregitope-blood component conjugates and/or the modified polypeptides used to form the Tregitope-blood component conjugates, or a pharmaceutical composition or formulation containing such. In aspects, the subject is a human.

In aspects, the present disclosure is directed to a method of stimulating, inducing, and/or expanding regulatory T-cells (e.g., naturally occurring T_{Regs} (in aspects, including natural T_{Regs} and/or adaptive T_{Regs})) to suppress an autoimmune response in a subject in need thereof by administering to the subject a therapeutically effect amount of the instantly disclosed Tregitope-blood component conjugates and/or the modified polypeptides used to form the Tregitope-blood component conjugates (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein). In aspects, the immune response is the result of one or more therapeutic treatments with at least one therapeutic protein, treatment with a vaccine or treatment with at least one antigen. In aspects, the administration of such shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method for expanding a population of regulatory T cells, comprising: (a) providing a biological sample from a subject; and (b) isolating regulatory T-cells from the biological sample; (c) contacting the isolated regulatory T-cells with an effective amount of the instantly disclosed Tregitope-blood component conjugates and/or the modified polypeptides used to form the Tregitope-blood component conjugates (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein), under conditions wherein the T-regulatory cells increase in number to yield an expanded regulatory T-cell composition, thereby expanding the regulatory T-cells in the biological sample; and, additionally, (d) returning the sample to the subject in need of treatment.

In aspects, the present disclosure is directed to a method for stimulating regulatory T cells in a biological sample, comprising: (a) providing a biological sample from a subject; (b) isolating regulatory T-cells from the biological sample; (c) contacting the isolated regulatory T-cells with an effective amount of the instantly disclosed Tregitope-blood component conjugates and/or the modified polypeptides used to form the Tregitope-blood component conjugates (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein), under conditions wherein the T-regulatory cells are stimulated to alter one or more biological function, thereby stimulating the regulatory T-cells in the biological sample; and, additionally, (d) returning cells to the subject in need of treatment.

In aspects, the present disclosure is directed to a method for repressing/suppressing an immune response in a subject, comprising administering a therapeutically effective amount of the instantly-disclosed Tregitope-blood component conjugates and/or the modified polypeptides used to form the Tregitope-blood component conjugates (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein), wherein the administered composition represses/suppresses the immune response. In aspects, the administered composition represses/suppresses an innate immune response. In aspects, the administered composition represses/suppresses an adaptive immune response. In aspects, the administered composition represses/suppresses an effector T cell response. In aspects, the administered composition represses/suppresses a memory T cell response. In aspects, the administered composition represses/suppresses helper T cell response. In aspects, the administered composition represses/suppresses B cell response. In aspects, the administered composition represses/suppresses an nkT cell (natural killer T cell) response. In another aspect, the administration of a Tregitope compound or composition of the present disclosure shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method of suppressing an immune response, specifically an antigen specific immune response in a subject, through the administration of a therapeutically effective amount of the instantly disclosed Tregitope-blood component conjugates and/or the modified polypeptides used to form the Tregitope-blood component conjugates (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein), wherein said administered composition activates naturally occurring T_{Regs} (in aspects, including natural T_{Regs} and/or adaptive T_{Regs}, and in aspects CD4⁺/CD25⁺/FoxP3⁺ regulatory T-cells) or suppresses the activation of CD4⁺ T-cells, the proliferation of CD4⁺ and/or CD8⁺ T-cells, and/or suppresses the activation or proliferation of β-cells or nkT Cells. In aspects, a Tregitope-blood component conjugates and/or the modified polypeptides used to form the Tregitope-blood component conjugates of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein) may be either covalently bound, non-covalently bound, or in admixture with a specific target antigen. In aspects, the specific target antigen is a T1Dgen included in the modified polypeptide. In aspects, an administered Tregitope-blood component conjugates and/or the modified polypeptides used to form the Tregitope-blood component conjugates of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein) that is covalently bound, non-covalently bound, or in admixture with one or more specific target antigens results in the diminution of immune response against the target antigen. In aspects, the one or more specific target antigens comprises, consists of, or consists essentially of one or more peptides associated with immunogenicity in diabetes (termed “T1Dgens”) having a sequence comprising, consisting of, or consisting essentially of one or more of SEQ ID NOS: 56-63 as disclosed herein.

In aspects, the target antigen may be an autologous protein or protein fragment. In aspects, the target antigen may be allogenic protein or protein fragments. In aspects, the target antigen may be a biologic medicine or fragments thereof. In aspects, the target antigen is a preproinsulin or fragments thereof. In aspects, the target antigen comprises, consists of, or consists essentially of one or more of SEQ ID NOS: 56-63, or fragments and variants thereof as described herein. In aspects, the suppressive effect is mediated by natural T_{Regs}. In aspects, the suppressive effect is mediated by adaptive T_{Regs}. In aspects, the one or more Tregitopes of the presently disclosed modified polypeptides suppresses an innate immune response. In aspects, the one or more Tregitopes of the presently disclosed modified polypeptides suppresses an adaptive immune response. In aspects, the one or more Tregitopes of the presently disclosed modified polypeptides suppresses helper T cell response. In aspects, the one or more Tregitopes of the presently disclosed modified polypeptides suppresses a memory T cell response. In aspects, the one or more Tregitopes of the presently disclosed modified polypeptides suppresses B cell response. In aspects, the one or more Tregitopes of the presently disclosed modified polypeptides suppresses nkT cell response.

In aspects, the present disclosure is directed to a kit for preventing or treating a medical condition, in particular, for the suppression of an immune response associated with Type 1 Diabetes in a subject, wherein the kit comprises an instantly disclosed Tregitope-blood component conjugate and/or one or more instantly disclosed modified polypeptides used to form the Tregitope-blood component conjugates (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein). In aspects, the kit may further comprise an effective amount of an antigen or therapeutic agent, such as a replacement protein or peptide.

### BRIEF DESCRIPTION OF THE DRAWINGS

The present disclosure may be better understood with reference to the following figures. The patent or application file contains at least one drawing executed in color. Copies of this patent or patent application publication with color drawing(s) will be provided by the Office upon request and payment of the necessary fee.

FIG. 1 shows an exemplary Tregitope-blood component conjugate of the instant disclosure. HSA refers to human serum albumin.

FIG. 2 shows an exemplary Tregitope-blood component conjugate of the instant disclosure. HSA refers to human serum albumin.

FIG. 3 shows an experimental design for a TTBSA assay evaluating the efficacy of Tregitope-albumin delivery vehicles.

FIGS. 4A and B show the results of each of a number of available Tregitopes individually and in pairwise combinations for their potential to suppress CD4+ T cell proliferation in the TTBSA assay.

FIGS. 5A and B show the results of conjugating selected Tregitopes for their potential to suppress CD4+ T cell proliferation in the TTBSA assay.

### DETAILED DESCRIPTION OF THE INVENTION

### General

The adaptive immune cascade begins when soluble protein antigens are taken up by Antigen Presenting Cells (APCs) and processed through the Class II antigen presentation pathway. Protein antigens in the Class II presentation pathway are degraded by various proteases found in the Endoplasmic Reticulum. Some of the resulting protein fragments are bound to Class II MHC molecules. Peptide-loaded MHC molecules are trafficked to the cell surface where they are interrogated by CD4+ T cells. Peptide fragments that are capable of binding to an MHC molecule and mediating the cell-to-cell interaction between APC and circulating T cells are referred to as T cell epitopes. Recognition of these peptide-MHC complexes by CD4+ T cells can lead to either an immune activating or immune suppressive response based on the phenotype of the responding T cells and the local cytokine/chemokine milieu. In general, engagement between the MHC/peptide complex and the T cell receptor (TCR) of T effector cells leads to activation and the subsequent secretion of pro-inflammatory cytokines such as IL-4, and IFN-□. On the other hand, the activation of natural T regulatory cells (T_{Regs}) leads to the expression of the immune suppressive cytokines IL-10 and TGF-1, among others (Shevach E, (2002), Nat Rev Immunol, 2(6):389-400). These cytokines act directly on nearby effector T cells leading in some cases to anergy or apoptosis. In other cases, regulatory cytokines and chemokines convert effector T cells to T regulatory phenotypes; this process is referred here as “induced” or “adaptive” tolerance. T cell epitopes that are capable of binding to MHC molecules and engaging and/or activating circulating naturally occurring T_{Regs} (in aspects, including natural T_{Regs} and/or adaptive T_{Regs}), are referred to as Tregitopes. In aspects, the instantly disclosed Tregitopes are T cell epitope clusters, which are epitopes capable of binding to multiple MHC alleles and multiple TCRs.

Initial self/non-self discrimination occurs in the thymus during neonatal development where cortical and medullary epithelial cells express specific self-protein epitopes to immature T cells. T cells recognizing self-antigens with high affinity are deleted, but autoreactive T cells with moderate affinity sometimes avoid deletion and can be converted to natural regulatory T cells (T_{Reg}) cells. These natural T_{Reg} cells are exported to the periphery and help to control a latent autoimmune response. Natural regulatory T cells are a critical component of immune regulation and self-tolerance.

Self-tolerance is regulated by a complex interplay between T cells, B cells, cytokines and surface receptors. T regulatory immune responses counterbalance T effector immune response to protein antigens (whether self or foreign). A tilt of the balance toward the autoreactive side, either by increasing the number and/or function of autoreactive T effector cells or by diminishing the number and/or function of T regulatory cells, is manifested as autoimmunity.

A second form of tolerance occurs in the periphery where mature T cells are converted to an ‘adaptive’ T_{Reg} phenotype upon activation via their T cell receptor in the presence of IL-10 and TGF-□, usually supplied by bystander T regulatory cells. The possible roles for these ‘adaptive’ T_{Reg} cells include dampening immune response following the successful clearance of an invading pathogen, controlling excessive inflammation caused by an allergic reaction, controlling excessive inflammation caused by low level or chronic infection, or possibly controlling inflammatory response targeting beneficial symbiotic bacteria and viruses. ‘Adaptive’ T_{Regs} may also play a role in suppressing immune response targeting human antibodies that have undergone somatic hypermutation (Chaudhry A et al., (2011), Immunity, 34(4):566-78).

T_{Reg} cells are also instrumental in B cell tolerance. B cells express a single low-affinity Fc receptor, FcyRIIB on their cell surface (Ravetch J V et al., (1986), Science, 234(4777):718-25). This receptor contains the immunoreceptor tyrosine-based inhibition motif sequence (ITIM) in its cytoplasmic domain. Co-ligation of Fc□RIIB and the B-cell receptor (BCR) by immune complexes act to trigger the tyrosine phosphorylation of the ITIM leading to the recruitment of the inositol phosphatase, SHIP, which inhibits BCR-triggered proliferation by interfering with the activation of MAP kinases and blocks phagocytosis by the dissociation of Burton's tyrosine kinase (Btk) from the cell membrane, which inhibits calcium influx into the cell. Fc□RIIB can also induce apoptosis independent of the ITIM. Upon homo-aggregation of Fc□RIIB by ICs, the association of Btk with the cell membrane is enhanced, thereby triggering an apoptotic response (Pearse R, et al., (1999), Immunity, 10(6):753-60). Expression of Fc□RIIB is highly variable and cytokine dependent. IL-4 and IL-10, which are expressed by activated Th2 and T_{Reg} cells, have been shown to act synergistically to enhance Fc□RIIB expression (Joshi T et al., (2006), Mol Immuno., 43(7):839-50), thus aiding in the suppression of a humoral response.

It is possible to exploit Tregitope specific T_{Reg} cells to suppress unwanted immune responses, and also to induce adaptive T_{Reg} to co-delivered proteins. This discovery has implications for the design of therapeutic regimens and antigen-specific therapies for transplantation, protein therapeutics, allergy, chronic infection, autoimmunity and vaccine design. Administration of a drug, a protein, or an allergen in conjunction with Tregitopes, including novel Tregitope-blood component conjugates and modified polypeptides comprising Tregitopes of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein) can suppress an effector immune response and can be used to deliberately manipulate the immune system toward tolerance.

The Tregitope-blood component conjugates and modified polypeptides comprising Tregitopes of the present disclosure of the present disclosure are useful in the selective engagement and activation of regulatory T cells. It is demonstrated herein that certain naturally occurring T_{Regs} (in aspects, including natural T_{Regs} and/or adaptive T_{Regs}), can be engaged, activated, and/or applied to the suppression of unwanted immune responses in both systemic and limited, disease-specific contexts. For example, certain human proteins circulating in the blood steam, such as immunoglobulins, contain T cell epitopes that relate to naturally occurring populations of regulatory T cells (in aspects, including natural T_{Regs} and/or adaptive T_{Regs}). In the course of normal immune surveillance, these proteins are taken up by professional APCs, such as dendritic cells or macrophages, and degraded. During the degradation process, some of the epitopes contained in these proteins are bound to MHC molecules, transported to the cell surface presented to regulatory T cells. Those cells, once activated by the APC, release cytokines and chemokines help to suppress autoimmune responses that would otherwise hinder the function of the extra cellular proteins. In aspects, the Tregitope compositions of the present disclosure can be used to engage and activate pre-existing populations of regulatory T cells to suppress an autoimmune response associated with T1D. Suppression of an autoimmune response associated with T1D may involve suppression of CD8+ and/or CD4+ auto-reactive T cells targeting pancreatic islet cell antigens presented by human leukocyte antigen (HLA) molecules, suppression of the destruction of pancreatic islet cells by T cells, expansion and/or activation of pancreatic islet cell antigen-specific Tregs, restoration of tolerance of pancreatic islet cells, and/or inducing tolerance to protein associated with T1D and pancreatic islet cells, such as preproinsulin.

By using the novel Tregitope-blood component conjugates and modified polypeptides comprising Tregitopes of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein), it is herein shown that such compositions can be used to suppress a variety of unwanted immune responses. In its simplest form, systemic application of the Tregitope-blood component conjugates and modified polypeptides comprising Tregitopes of the present disclosure can be used as a generalized immune suppressant useful for controlling severe autoimmune reactions such as, for example, autoimmune T1D.

In a more controlled application, the novel Tregitope-blood component conjugates and modified polypeptides comprising Tregitopes of the present disclosure can be used to suppress localized autoimmune responses. In a targeted application, such as might be achieved through the linking or admixture of the Tregitope-blood component conjugates and modified polypeptides comprising Tregitopes of the present disclosure to certain other T cell epitopes, the instantly disclosed compositions can suppress highly specific immune reactions to the linked or admixed T cell epitopes while leaving the balance of the immune system intact. For example, through the delivery of a Tregitope-blood component conjugates and/or modified polypeptides, both comprising an autoimmune antigen such as preproinsulin or insulin (e.g., one or more T1Dgens having a sequence comprising, consisting of, or consisting essentially of one or more of SEQ ID NOS: 56-63), the immune system can be trained to “tolerate” the co-delivered antigen by, e.g., inducing naturally occurring T_{Regs} (in aspects, including natural T_{Regs} and/or adaptive T_{Regs}) and/or converting the phenotype of responding effector T cells to that of adaptive regulatory T cells that can suppress an immune response targeting the preproinsulin, insulin, or other proteins associated with pancreatic islet cells and/or T1D. Such immune reprogramming could reduce and/or eliminate immune response targeting pancreatic islet cells, insulin, preproinsulin, and other antigens associated with the autoimmune response in T1D, while leaving the balance of the immune system intact.

The Tregitopes of the present disclosure are derived from circulating extracellular proteins. To be useful, these Tregitopes should be true T cell epitopes (i.e., capable of binding to both MHC molecules and TCRs). In aspects, the Tregitopes should be related to a pre-existing population of regulatory T cells that is sufficiently large to have a therapeutic effect. T cell epitope clusters, which are epitopes capable of binding to multiple MHC alleles and multiple TCRs, are key to satisfying this latter qualification.

In their natural state, the Tregitopes of the present disclosure are capable of engaging and activating naturally occurring T_{Regs} (in aspects, including natural T_{Regs} and/or adaptive T_{Regs}) which prevent or terminate immune responses. Additionally, treatment with Tregitope-blood component conjugates and modified polypeptides comprising Tregitopes of the present disclosure can expand corresponding naturally occurring T_{Reg} populations (in aspects, including natural T_{Regs} and/or adaptive T_{Regs}), making them available to be activated by homologous peptides derived from insulin or preproinsulin, thereby suppressing effector response targeting insulin or preproinsulin. The instantly disclosed treatments provides the following advantages:

1. Treatment with the Tregitope compositions of the present disclosure is highly antigen-specific (e.g., treatment with the Tregitope compositions can, e.g., expand and/or stimulate corresponding naturally occurring T_{Reg} populations (in aspects, including natural T_{Regs} and/or adaptive T_{Regs}) in a highly antigen-specific manner);

2. An efficient and less expensive treatment regimen when compared to current antigen-specific therapies for T1D; and

3. Prevention or treatment of T1D, such that subjects maintain tolerance and/or become tolerant to glucose, and/or need not depend on insulin replacement therapies.

In aspects, the present disclosure provides novel Tregitope-blood component conjugates and modified polypeptides comprising Tregitopes, with said modified peptides being capable of reacting with blood components to form such Tregitope-blood component conjugates. Conjugation of a polypeptide comprising a Tregitope with a blood component, such as albumin, can be useful as a carrier protein for a Tregitope payload. Tregitope-blood component conjugates can extend the half-life of the modified polypeptides comprising Tregitopes in vivo, protect the modified polypeptides comprising Tregitopes from rapid proteolytic degradation, protect the modified polypeptides comprising Tregitopes from rapid clearance from circulation and/or rapid kidney excretion, allow for wide distribution of Tregitope-blood component conjugates throughout the body of a subject, aid in delivery of modified polypeptides comprising Tregitopes to appropriate immune cells (such as macrophages and APCs), allow the modified polypeptides comprising Tregitopes to be processed by the endocytic pathway of certain immune cells (such as macrophages and APCs), and/or aid in the presentation of modified polypeptides comprising Tregitopes as an antigen by said immune cells

### Definitions

To further facilitate an understanding of the present invention, a number of terms and phrases are defined below. Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction

As used herein, the term “biological sample” as refers to any sample of tissue, cells, or secretions from an organism.

As used herein, the term “transplantation” refers to the process of taking a cell, tissue, or organ, called a “transplant” or “graft” from one subject and placing it or them into a (usually) different subject. The subject who provides the transplant is called the “donor”, and the subject who received the transplant is called the “recipient”. An organ or graft transplanted between two genetically different subjects of the same species is called an “allograft”. A graft transplanted between subjects of different species is called a “xenograft”.

As used herein, the term “medical condition” includes, but is not limited to, any condition or disease manifested as one or more physical and/or psychological symptoms for which treatment and/or prevention is desirable, and includes previously and newly identified diseases and other disorders.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens, cells or tissues infected with pathogens, or, in cases of autoimmunity or pathological inflammation, normal human cells or tissues. In aspects, an immune response includes a measurable cytotoxic T lymphocyte (CTL) response (e.g., against a virus expressing an immunogenic polypeptide) or a measurable B cell response, such as the production of antibodies, (e.g., against an immunogenic polypeptide). One of ordinary skill would know various assays to determine whether an immune response against a peptide, polypeptide, or related composition was generated, including use of the experiments and assays as disclosed in the Examples herein. Various B lymphocyte and T lymphocyte assays are well known, such as ELISAs, EliSpot assays, cytotoxic T lymphocyte CTL assays, such as chromium release assays, proliferation assays using peripheral blood lymphocytes (PBL), tetramer assays, and other cytokine production assays. See Benjamini et al. (1991), hereby incorporated by reference.

As used herein, the term “effective amount”, “therapeutically effective amount”, or the like of a composition, including Tregitope-blood component conjugates and modified polypeptides comprising Tregitopes (with said modified peptides being capable of reacting with blood components to form such Tregitope-blood component conjugates) of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein) is a quantity sufficient to achieve a desired therapeutic and/or prophylactic effect, e.g., an amount that results in the prevention of, or a decrease in, the symptoms associated with a disease that is being treated. The amount of a composition of the present disclosure administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present invention can also be administered in combination with each other or with one or more additional therapeutic compounds.

As used herein, the term “regulatory T cell”, “Treg” or the like, means a subpopulation of T cells that suppress immune effector function, including the suppression or down regulation of CD4+ and/or CD8+ effector T cell (Teff) induction, proliferation, and/or cytokine production, through a variety of different mechanisms including cell-cell contact and suppressive cytokine production. In aspects, CD4+ Tregs are characterized by the presence of certain cell surface markers including but not limited to CD4, CD25, and FoxP3. In aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, CD8+ Tregs may also exert immune suppressive effects through direct killing of target cells, characterized by the expression upon activation of effector molecules including but not limited to granzyme B and/or perforin.

As used herein, the term “T cell epitope” means an MHC ligand or protein determinant, 7 to 30 amino acids in length, and capable of specific binding to human leukocyte antigen (HLA) molecules and interacting with specific T cell receptors (TCRs). As used herein, in the context of a T cell epitope that is known or determined (e.g. predicted) to engage a T cell, the terms “engage”, “engagement” or the like means that when bound to a MHC molecule (e.g. human leukocyte antigen (HLA) molecules), the T cell epitope is capable of interacting with the TCR of the T cell and activating the T cell. Generally, T cell epitopes are linear and do not express specific three-dimensional characteristics. T cell epitopes are not affected by the presence of denaturing solvents. The ability to interact with T cell epitopes can be predicted by in silico methods (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A R et al., (2003), Vaccine, 21(27-30):4486-504, all of which are herein incorporated by reference in their entirety).

As used herein, the term “T-cell epitope cluster” refers to polypeptide that contains between about 4 to about 40 MHC binding motifs. In particular embodiments, the T-cell epitope cluster contains between about 5 to about 35 MHC binding motifs, between about 8 and about 30 MHC binding motifs, or between about 10 and 20 MHC binding motifs.

As used herein, the term “regulatory T cell epitope” (“Tregitope”) refers to a “T cell epitope” that causes a tolerogenic response (Weber C A et al., (2009), Adv Drug Deliv, 61(11):965-76) and is capable of binding to MHC molecules and engaging (i.e. interacting with and activating) circulating naturally occurring Tregs (in aspects, including natural Tregs and/or adaptive Tregs). In aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin, leads to the expression of the immune suppressive cytokines including, but not limited to, IL-10 and TGF-β and TNF-α. In aspects, upon activation, regulatory CD8+ T cells secrete immune suppressive cytokines and chemokines including but not limited to IFNγ, IL-10, and/or TGFβ. In aspects, CD8+ Tregs may also exert immune suppressive effects through direct killing of target cells, characterized by the expression upon activation of effector molecules including but not limited to granzyme B and/or perforin. In aspects, the instantly disclosed Tregitopes are T cell epitope clusters, which are epitopes capable of binding to multiple MHC alleles and multiple TCRs.

As used herein, the term “immune stimulating T-cell epitope polypeptide” refers to a molecule capable of inducing an immune response, e.g., e.g., a humoral, T cell-based, or innate immune response. In aspects, an immune stimulating T-cell epitope polypeptide is human Coagulation Factor V or Coagulation Factor VIII.

As used herein, the term “B cell epitope” means a protein determinant capable of specific binding to an antibody. B cell epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the terms “the major histocompatibility complex (MHC)”, “MHC molecules”, “MHC proteins” or “HLA proteins” are to be understood as meaning, in particular, proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles. MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin. MHC molecules of class II consist of an α-chain and a β-chain and are capable of binding a peptide of about 12 to 25 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

As used herein, the term “MHC complex” refers to a protein complex capable of binding with a specific repertoire of polypeptides known as HLA ligands and transporting said ligands to the cell surface.

As used herein, the term “MHC Ligand” means a polypeptide capable of binding to one or more specific MHC alleles. The term “HLA ligand” is interchangeable with the term “MHC Ligand”. Cells expressing MHC/Ligand complexes on their surface are referred to as “Antigen Presenting Cells” (APCs). Similarly, as used herein, the term “MHC binding peptide” relates to a peptide which binds to an MHC class I and/or an MHC class II molecule. In the case of MHC class I/peptide complexes, the binding peptides are typically 8-10 amino acids long although longer or shorter peptides may be effective. In the case of MHC class II/peptide complexes, the binding peptides are typically 10-25 amino acids long and are in particular 13-18 amino acids long, whereas longer and shorter peptides may also be effective.

As used herein, the term “T Cell Receptor” or “TCR” refers to a protein complex expressed by T cells that is capable of engaging a specific repertoire of MHC/Ligand complexes as presented on the surface of cells, such as antigen presenting cells (APCs).

As used herein, the term “MHC Binding Motif” refers to a pattern of amino acids in a protein sequence that predicts binding to a particular MHC allele.

As used herein, the term “EpiBar™” refers to a single 9-mer frame that is predicted to bind to at least four different HLA alleles. Cluster scores higher than 10 are considered to be significant. All scores in the top 5% are considered “hits”.

As used herein, the term “Immune Synapse” means the protein complex formed by the simultaneous engagement of a given T cell epitope to both a cell surface MHC complex and TCR.

The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide (e.g., a polypeptide comprising, consisting of, or consisting essentially of one or more of SEQ ID NOS: 1-55 (and variants and fragments thereof as disclosed herein), one or more peptides associated with immunogenicity in diabetes (termed “T1Dgens”) having a sequence comprising, consisting of, or consisting essentially of one or more of SEQ ID NOS: 56-63 (and variants and fragments thereof as disclosed herein), however, can be joined to, linked to, or inserted into another polypeptide (e.g., a heterologous polypeptide) with which it is not normally associated in a cell and still be “isolated” or “purified.”

As used herein, the term “pharmaceutically acceptable” refers to approved or approvable by a regulatory agency of the Federal or a state government or listed in the U.S. Pharmacopeia or other generally recognized pharmacopeia for use in animals, including humans.

As used herein, the term “pharmaceutically acceptable excipient, carrier, or diluent” or the like refer to an excipient, carrier, or diluent that can be administered to a subject, together with an agent, and which does not destroy the pharmacological activity thereof and is nontoxic when administered in doses sufficient to deliver a therapeutic amount of the agent.

As used herein, a “free thiol” refers to a thiol side chain of an amino acid optionally in a polypeptide and/or protein, wherein the thiol contains a sulfhydryl group. For example, free thiols are not bound to the side chains of other amino acids through intramolecular or intermolecular disulfide bonds.

As used herein, “functionalities” are groups on blood components, including mobile and fixed proteins, to which reactive groups on modified therapeutic peptides react to form covalent bonds. Functionalities may include hydroxyl groups for bonding to ester reactive groups, thiol groups for bonding to maleimides, imidates and thioester groups; amino groups for bonding to activated carboxyl, phosphoryl or any other acyl groups on reactive groups.

As used herein, “blood components” may be either fixed or mobile. Fixed blood components are non-mobile blood components and include tissues, membrane receptors, interstitial proteins, fibrin proteins, collagens, platelets, endothelial cells, epithelial cells and their associated membrane and membranous receptors, somatic body cells, skeletal and smooth muscle cells, neuronal components, osteocytes and osteoclasts and all body tissues especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1 μg/ml. Mobile blood components include serum albumin, transferrin, ferritin and immunoglobulins such as IgM and IgG. The half-life of mobile blood components may be at least about 12 hours.

As used herein, the term “purpose built computer program” refers to a computer program designed to fulfill a specific purpose; typically to analyze a specific set of raw data and answer a specific scientific question.

As used herein, the term “z-score” indicates how many standard deviations an element is from the mean. A z-score can be calculated from the following formula. z=(X−μ)/σ, where z is the z-score, X is the value of the element, μ is the population mean, and σ is the standard deviation.

As used herein, the singular forms “a,” “an,” and “the” are intended to include the plural forms, including “at least one,” unless the content clearly indicates otherwise. “Or” means “and/or.” As used herein, the term “and/or” and “one or more” includes any and all combinations of the associated listed items. For example, the term “one or more” with respect to the “one or more of SEQ ID NOS: 1-55 of the present disclosure” includes any and all combinations of SEQ ID NOS: 1-55. The term “or a combination thereof” means a combination including at least one of the foregoing elements.

The following abbreviations and/or acronyms are used throughout this application:
APC antigen presenting cells
CEF cytomegalovirus, Epstein-Barr virus and influenza virus
CFSE dye carboxyfluorescein succinimidyl ester dye
DMSO dimethyl sulfoxide
DR antibody antigen D related antibody
ELISA enzyme-linked immunosorbent assay
FACS fluorescence-activated cell sortings
Fmoc 9-fluoronyl methoxy carbonyl
FV human coagulation Factor V
FVIII human coagulation Factor VIII
HLA human leukocyte antigen
HPLC high-performance liquid chromatography
IVIG intravenous purified Immunoglobulin G antibody
MFI mean fluorescence index
MHC major histocompatibility complex
PBMC peripheral blood mononuclear cell
PI proliferation index
RPMI Roswell Park Memorial Institute medium
Teff effector T cell
T_{Reg} regulatory T cell
TT tetanus toxoid
UV ultraviolet

As used herein, a “variant” peptide or polypeptide (including a variant Tregitope) can differ in amino acid sequence by one or more substitutions, deletions, insertions, inversions, fusions, and truncations or a combination of any of these. In aspects, a variant polypeptide can differ in amino acid sequence by one or more substitutions, deletions, insertions, inversions, fusions, and truncations or a combination of any of these, provided said variants retain MHC binding propensity and/or TCR specificity, and in aspects when the peptide or polypeptide comprises a Tregitope, and/or regulatory T cell stimulating or suppressive activity.

The present disclosure also includes polypeptide fragments of the Tregitopes and other peptides or polypeptides as described herein. The invention also encompasses fragments of the variants of the Tregitopes and other peptides or polypeptides described herein, provided said fragments and/or variants retain MHC binding propensity and/or TCR specificity and in aspects when the peptide or polypeptide comprises a Tregitope, and/or regulatory T cell stimulating or suppressive activity.

An “isolated” peptide or polypeptide (e.g., an isolated T_{reg} activating regulatory T-cell epitope, Tregitope, or T-cell epitope polypeptide) can be purified from cells that naturally express it, purified from cells that have been altered to express it (recombinant), or synthesized using known protein synthesis methods. In one embodiment, a Tregitope is produced by recombinant DNA or RNA techniques. For example, a nucleic acid molecule encoding the Tregitope is cloned into an expression vector, the expression vector introduced into a host cell and the polypeptide expressed in the host cell. The Tregitope can then be isolated from the cells by an appropriate purification scheme using standard protein purification techniques.

For the purposes of the present disclosure, Tregitopes and other peptides or polypeptides of the instant disclosure can include, for example, modified forms of naturally occurring amino acids such as D-stereoisomers, non-naturally occurring amino acids; amino acid analogs; and mimetics. Further, in aspects, Tregitopes and other peptides or polypeptides of the instant disclosure can include retro-inverso peptides, provided said retro-inverso peptides or polypeptides of the instant disclosure at least in part retain MHC binding propensity and/or TCR specificity, and in aspects when the peptide or polypeptide comprises a Tregitope, and/or regulatory T cell stimulating or suppressive activity.

Although any methods and materials similar or equivalent to those described herein can be used in the practice or testing of the present disclosure, the preferred methods and materials are described. Other features, objects, and advantages of the present disclosure will be apparent from the description and the Claims. In the Specification and the appended Claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entirety and for all purposes to the same extent as if each individual publication, patent, or patent application was specifically and individually indicated to be incorporated by reference in its entirety for all purposes.

### Tregitope-Blood Component Conjugates and Modified Polypeptides Comprising Tregitopes

The aim of the present disclosure is to provide novel Tregitope-blood component conjugates and modified polypeptides comprising Tregitopes, with said modified peptides being capable of reacting with blood components to form such Tregitope-blood component conjugates. Conjugation of a polypeptide comprising a Tregitope with a blood component, such as albumin, can be useful as a carrier protein for a Tregitope payload. Tregitope-blood component conjugates can extend the half-life of the modified polypeptides comprising Tregitopes in vivo, protect the modified polypeptides comprising Tregitopes from rapid proteolytic degradation, protect the modified polypeptides comprising Tregitopes from rapid clearance from circulation and/or rapid kidney excretion, allow for wide distribution of Tregitope-blood component conjugates throughout the body of a subject, aid in delivery of modified polypeptides comprising Tregitopes to appropriate immune cells (such as macrophages and APCs), allow the modified polypeptides comprising Tregitopes to be processed by the endocytic pathway of certain immune cells (such as macrophages and APCs), and/or aid in the presentation of modified polypeptides comprising Tregitopes as an antigen by said immune cells.

The selective engagement and activation of naturally occurring Tregs (in aspects, including natural Tregs and/or adaptive Tregs) through the use of Tregitope-blood component conjugates and modified polypeptides comprising Tregitopes as disclosed herein, is therapeutically valuable as a means of treatment for a disease or condition marked by the presence of an unwanted immune response, for example autoimmune disease such as type 1 diabetes. As such, the present disclosure also relates to methods of using said Tregitope-blood component conjugates and modified polypeptides comprising Tregitopes in the treatment and prevent of autoimmune disorders, such as type 1 diabetes.

In aspects, the Tregitope-blood component conjugates comprise a blood component which acts as a carrier protein (e.g., albumin), and further comprise a modified polypeptide, said modified polypeptide comprising one or more regulatory T cell epitopes (termed “Tregitopes”). The modified polypeptide comprises a reactive moiety that is attached to the polypeptide, with the reactive moiety being capable of forming a bond (e.g., a covalent linkage) with a reactive functionality on the blood component. In aspects, the modified polypeptide further comprises one or more antigen peptides associated with immunogenicity in diabetes (“T1Dgen” peptides; e.g., PPI-derived peptides), which may be optionally separated from one or more Tregitopes by a linker and/or cleavage site (e.g., a lysosomal cleavage site). Tregitope-blood component conjugates may be formed by modifying a polypeptide comprising a Tregitope by attaching a reactive moiety to the polypeptide to create a modified polypeptide, then forming a bond between reactive moiety of the modified polypeptide with a reactive functionality on a blood component, as disclosed in U.S. Pat. Nos. 6,849,714, 6,887,470, 7,256,253, and 7,307,148, herein incorporated by reference in their entireties. In aspects of instantly disclosed Tregitope-blood component conjugates and modified polypeptides comprising Tregitopes, the Tregitope-blood component conjugates and modified polypeptides comprising Tregitopes may be isolated, synthetic, or recombinant.

In aspects, the blood components of the Tregitope-blood component conjugates may be either fixed or mobile, as disclosed in U.S. Pat. Nos. 6,849,714, 6,887,470, 7,256,253, and 7,307,148. Fixed blood components are non-mobile blood components and include tissues, membrane receptors, interstitial proteins, fibrin proteins, collagens, platelets, endothelial cells, epithelial cells and their associated membrane and membranous receptors, somatic body cells, skeletal and smooth muscle cells, neuronal components, osteocytes and osteoclasts and all body tissues, especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1 μg/ml. Mobile blood components include serum albumin, transferrin, ferritin and immunoglobulins such as IgM and IgG. The half-life of mobile blood components is at least about 12 hours. In aspects of the Tregitope-blood component conjugates, the blood component is albumin, such as serum albumin, human serum albumin, recombinant albumin, and recombinant human serum albumin. Albumin is a preferred blood component because it contains an Fc neonatal binding domain that will carry the Tregitope-albumin conjugate into the appropriate cells, such as macrophages and APCs. Further, albumin contains a cysteine at amino acid 34 (Cys34) (the location of the amino acid in the amino acid sequence of human serine albumin), containing a free thiol with a pKa of approximately 5, which may serve as a preferred reactive functionality of albumin. Cys34 of albumin is capable of forming a stable thioester bond with maleimidopropionamido (MPA), which is a preferred reactive moiety of a modified Tregitope peptide.

In aspects, reactive functionalities on the blood component of the Tregitope-blood component conjugates or on the blood components that are capable of forming a conjugate with the instantly disclosed modified polypeptides are groups on blood components, including mobile and fixed proteins, to which reactive groups on modified therapeutic peptides react to form covalent bonds. As disclosed in U.S. Pat. Nos. 6,849,714, 6,887,470, 7,256,253, and 7,307,148, such functionalities usually include hydroxyl groups for bonding to ester reactive groups, thiol groups for bonding to maleimides, imidates and thioester groups; amino groups for bonding to activated carboxyl, phosphoryl or any other acyl groups on reactive groups. In aspects, the reactive functionality of the blood component is an amino group, a hydroxyl group, or a thiol group. In aspects, the reactive functionality of the blood component is a component of a side group of an amino acid in a polypeptide and/or protein, wherein the reactive functionality is near the surface of the polypeptide and/or protein. In aspects, the reactive functionality of the blood component is a thiol group of a free cysteine residue of a proteinaceous blood component. In aspects, the reactive functionality is a free thiol group of the cysteine at amino acid 34 (Cys³⁴) of serine albumin. In aspects, the reactive functionality of the blood component is a thiol with a pKa of approximately 5 in a physiological environment, such as plasma. In aspects, the reactive functionality of the blood component is a thiol with a pKa of approximately 5.5 in a physiological environment, such as plasma. In aspects, the reactive functionality of the blood component is a thiol with a pKa of 3-7 in a physiological environment, such as plasma. In aspects, the reactive functionality of the blood component is a thiolate anion. In aspects, the reactive functionality is a thiolate anion of the cysteine at amino acid 34 (Cys³⁴) of serine albumin.

In aspects, the modified polypeptides of the Tregitope-blood component conjugates and the modified polypeptides used to form the Tregitope-blood component conjugates comprise a reactive moiety that is attached to the polypeptide, with the reactive moiety being capable of forming a bond (e.g., a covalent linkage) with a reactive functionality on the blood component. In aspects, the reactive group is capable of reacting with an amino group, a hydroxyl group, or a thiol group on blood component to form a covalent bond therewith. In aspects, the reactive group is placed at a site such that when the modified polypeptide is bonded to the blood component, the modified peptide retains a substantial proportion of the parent compound's activity. In aspects, the reactive moiety may be a succinimidyl or maleimido group. In aspects, the reactive moiety may be attached to an amino acid positioned in the less therapeutically active region of amino acids of the polypeptide to be modified. In aspects, the reactive moiety is attached to the amino terminal amino acid of the modified polypeptide. In aspects, the reactive moiety is attached to the carboxy terminal amino acid of the modified polypeptide. In aspects, the reactive moiety is attached to an amino acid positioned between the amino terminal amino acid and the carboxy terminal amino acid of the modified polypeptide. In aspects, the reactive group may be attached to the polypeptide (to be modified) either via a linking group, or optionally without using a linking group. Further, one or more additional amino acids (e.g., one or more lysines) may be added to the polypeptide to facilitate the attachment of the reactive group. Linking groups are chemical moieties that link or connect reactive groups of blood components to polypeptides comprising one or more Tregitopes. Linking groups may comprise one or more alkyl groups, alkoxy group, alkenyl group, alkynyl group or amino group substituted by alkyl groups, cycloalkyl group, polycyclic group, aryl groups, polyaryl groups, substituted aryl groups, heterocyclic groups, and substituted heterocyclic groups. Linking groups may also comprise poly ethoxy aminoacids such as AEA ((2-amino)ethoxy acetic acid) or a preferred linking group AEEA ([2-(2-amino)ethoxy)]ethoxy acetic acid). In aspects, linking groups may comprise a polyethylene glycol linker (e.g. but not limited to, PEG2 or PEG12).

As should be understood, modified polypeptides may be administered in vivo such that conjugation with blood components occurs in vivo, or they may be first conjugated to blood components in vitro and the resulting peptidase-stabilized polypeptide administered in vivo. Further, as disclosed in in U.S. Pat. Nos. 6,849,714, 6,887,470, 7,256,253, and 7,307,148, a peptidase-stabilized polypeptide is a modified polypeptide that has been conjugated to a blood component via a covalent bond formed between the reactive group of the modified peptide and the functionalities of the blood component, with or without a linking group. Such reaction is preferably established by covalent bonding of a polypeptide modified with a maleimide link (e.g. prepared from GMBS, MPA or other maleimides) to a thiol group on a mobile blood protein such as serum albumin or IgG. Peptidase-stabilized polypeptides are more stable in the presence of peptidases in vivo than a non-stabilized peptide. A peptidase-stabilized therapeutic peptide generally has an increased half-life of at least 10-50% as compared to a non-stabilized peptide of identical sequence. Peptidase-stability is determined by comparing the half-life of the unmodified therapeutic peptide in serum or blood to the half-life of a modified counterpart therapeutic peptide in serum or blood. Half-life is determined by sampling the serum or blood after administration of the modified and non-modified peptides and determining the activity of the peptide. In addition to determining the activity, the length of the therapeutic peptide may also be measured.

In aspects, the modified polypeptides of the Tregitope-blood component conjugates and the modified polypeptides used to form the Tregitope-blood component conjugates comprise one or more Tregitopes (which may be termed herein as “Treg activating regulatory T-cell epitope”, “Tregitope”, or “T-cell epitope polypeptide”), which comprise a peptide or polypeptide chain derived from common human proteins. Tregitopes of the present disclosure are highly conserved among known variants of their source proteins (e.g., present in more than 10% of known variants). Tregitopes of the present disclosure comprise at least one putative T cell epitope as identified by EpiMatrix™ analysis. EpiMatrix™ is a proprietary computer algorithm developed by EpiVax (Providence, R.I.), which is used to screen protein sequences for the presence of putative T cell epitopes. Input sequences are parsed into overlapping 9-mer frames where each frame overlaps the last by 8 amino acids. Each of the resulting frames is then scored for predicted binding affinity with respect to a panel of eight common Class II HLA alleles (DRB1*0101, DRB1*0301, DRB1*0401, DRB1*0701, DRB1*0801, DRB1*1101, DRB1*1301, and DRB1*1501). Raw scores are normalized against the scores of a large sample of randomly generated peptides. The resulting “Z” score is reported. In aspects, any 9-mer peptide with an allele-specific EpiMatrix™ Z-score in excess of 1.64, theoretically the top 5% of any given sample, is considered a putative T cell epitope.

Peptides containing clusters of putative T cell epitopes are more likely to test positive in validating in vitro and in vivo assays. The results of the initial EpiMatrix™ analysis are further screened for the presence of putative T cell epitope “clusters” using a second proprietary algorithm known as Clustimer™ algorithm. The Clustimer™ algorithm identifies sub-regions contained within any given amino acid sequence that contains a statistically unusually high number of putative T cell epitopes. Typical T-cell epitope “clusters” range from about 9 to roughly 30 amino acids in length and, considering their affinity to multiple alleles and across multiple 9-mer frames, can contain anywhere from about 4 to about 40 putative T cell epitopes. Each epitope cluster identified an aggregate EpiMatrix™ score is calculated by summing the scores of the putative T cell epitopes and subtracting a correcting factor based on the length of the candidate epitope cluster and the expected score of a randomly generated cluster of the same length. EpiMatrix™ cluster scores in excess of +10 are considered significant. In aspects, the Tregitopes of the instant disclosure contain several putative T cell epitopes forming a pattern known as a T cell epitope cluster.

Many of the most reactive T cell epitope clusters contain a feature referred to as an “EpiBar™”. As previously described, an EpiBar™ is a single 9-mer frame that is predicted to be reactive to at least four different HLA alleles. In aspects, the Tregitopes of the present disclosure can comprise one or more EpiBars™.

The JanusMatrix system (EpiVax, Providence, R.I.) useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in the therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score above 3.0 indicate high tolerogenicity potential and as such may be very useful Tregitopes of the present disclosure.

In aspects, Tregitopes of the present disclosure bind to at least one and preferably two or more common HLA class II molecules with at least a moderate affinity (e.g., in aspects, <1000 μM IC50, <500 μM IC50, <400 μM IC50, <300 μM IC50, or <200 μM IC₅₀ in HLA binding assays based on soluble HLA molecules). In aspects, Tregitopes of the present disclosure are capable of being presented at the cell surface by APCs in the context of at least one and, in other aspects, two or more alleles of the HLA. In this context, the Tregitope HLA complex can be recognized by naturally occurring TRegs (in aspects, including natural TRegs and/or adaptive TRegs) having TCRs that are specific for the Tregitope HLA complex and circulating in normal control subjects. In aspects, the recognition of the Tregitope-HLA complex can cause the matching regulatory T cell to be activated and to secrete regulatory cytokines and chemokines.

In aspects, the one or more Tregitopes of the modified polypeptides have a sequence comprising, consisting of, or consisting essentially of one or more of SEQ ID NOS: 1-55 (and fragments and variants thereof). The phrase “consisting essentially of” is intended to mean that a Tregitope of the modified polypeptides according to the present disclosure, in addition to the sequence according to any of SEQ ID NOS: 1-55 (or a fragment or variant thereof), contains additional amino acids or residues that may be present at either terminus of the peptide and/or on a side chain that are not necessarily forming part of the peptide that functions as an MHC ligand and provided they do not substantially impair the activity of the peptide to function as a Tregitope. In aspects, the peptides or polypeptides of the instant disclosure can be either in neutral (uncharged) or salt forms, and may be either free of or include modifications such as glycosylation, side chain oxidation, or phosphorylation. In certain aspects, such polypeptides can be capped with an N-terminal acetyl and/or C-terminal amino group. In aspects, the Tregitope of the modified polypeptides according to the present disclosure comprises, consists of, or consists essentially of one or more of SEQ ID NOS: 1 and 28. In aspects, the Tregitope of the modified polypeptides according to the present disclosure comprises, consists of, or consists essentially of the amino acid sequence of SEQ ID NO: 1. In aspects, the Tregitope of the modified polypeptides according to the present disclosure comprises, consists of, or consists essentially of the amino acid sequence of SEQ ID NO: 28. In aspects, the one or more Tregitopes of the modified polypeptide may optionally have one or more linkers, which may optionally be cleavage sensitive sites, adjacent to their N-terminal and/or C-terminal end. In such a modified polypeptide, two or more of the Tregitopes may have a cleavage sensitive site between them. Alternatively, two or more of the Tregitopes may be connected directly to one another or through a linker that is not a cleavage sensitive site. In aspects, the modified polypeptides of the Tregitope-blood component conjugates and the modified polypeptides used to form the Tregitope-blood component conjugates include one or more of the following regulatory Tregitopes (as well as fragments thereof, variants thereof, and fragments of such variants, provided said fragments and/or variants retain MHC binding propensity and TCR specificity):

| | |
|---|---|
| SEQ ID NO: 1: | EEQYNSTYRVVSVLTVLHQDW |
| SEQ ID NO: 2: | PAVLQSSGLYSLSSVVTVPSSSLGTQ |
| SEQ ID NO: 3: | PGLVRPSQTLSLTCT |
| SEQ ID NO: 4: | GGLVQPGGSLRLSCAASGFTF |
| SEQ ID NO: 5: | GGLVQPGRSLRLSCAASGFTF |
| SEQ ID NO: 6: | GASVKVSCKASGYTF |
| SEQ ID NO: 7: | WSWVRQPPGRGLEWI |
| SEQ ID NO: 8: | WSWIRQPPGKGLEWI |
| SEQ ID NO: 9: | MHWVRQAPGKGLEWV |
| SEQ ID NO: 10: | MHWVRQAPGQGLEWM |
| SEQ ID NO: 11: | VDTSKNQFSLRLSSVTAADTA |
| SEQ ID NO: 12: | NTLYLQMNSLRAEDTAVYYCA |
| SEQ ID NO: 13: | FQHWGQGTLVTVSS |
| SEQ ID NO: 14: | FDLWGRGTLVTVSS |
| SEQ ID NO: 15: | FDIWGQGTMVTVSS |
| SEQ ID NO: 16: | FDYWGQGTLVTVSS |
| SEQ ID NO: 17: | FDPWGQGTLVTVSS |
| SEQ ID NO: 18: | MDVWGQGTLVTVSS |
| SEQ ID NO: 19: | MDVWGQGTTVTVSS |
| SEQ ID NO: 20: | LNNFYPREAKVQWKVDNALQSGNS |
| SEQ ID NO: 21: | KVYACEVTHQGLSS |
| SEQ ID NO: 22: | DIQMTQSPSSLSA |
| SEQ ID NO: 23: | EIVLTQSFGTLSL |
| SEQ ID NO: 24: | GDRVTITCRASQGIS |
| SEQ ID NO: 25: | LAWYQQKPGKAPKL |
| SEQ ID NO: 26: | LAWYQQKPGQAPRL |
| SEQ ID NO: 27: | LLIYGASSRATGIPD |
| SEQ ID NO: 28: | GTDFTLTISSLQPED |
| SEQ ID NO: 29: | SYELTQPPSVSVS |
| SEQ ID NO: 30: | GQSITISCTGTSSDV |
| SEQ ID NO: 31: | VSWYQQHPGKAPKL |
| SEQ ID NO: 32: | VHWYQQKPGQAPVL |
| SEQ ID NO: 33: | VSWYQQLPGTAPKL |
| SEQ ID NO: 34: | LMIYEVSNRPSGVPD |
| SEQ ID NO: 35: | LKKYLYEIARRHPYFYAPE |
| SEQ ID NO: 36: | APELLFFAKRYKAAFTECCQAA |
| SEQ ID NO: 37: | HPDYSVVLLLRLAKTYETTLE |
| SEQ ID NO: 38: | HPDYSVYLLLRLAKT |
| SEQ ID NO: 39: | LLLRLAKTYETTLE |
| SEQ ID NO: 40: | LGEYKFQNALLVRYTKKVPQVSTPT |
| SEQ ID NO: 41: | PADVAIQLTFLRLMSTEASQNI |
| SEQ ID NO: 42: | TGNLKKALLLQGSNEIEIR |
| SEQ ID NO: 43: | DGDFYRADQPRSAPSL |
| SEQ ID NO: 44: | SKEMATQLAFMRLLANYASQNITYH |
| SEQ ID NO: 45: | VQHIQLLQKNVRAQLVDMK |
| SEQ ID NO: 46: | GEFWLGNDYLHLLTQRQSVLRVE |
| SEQ ID NO: 47: | QSGLYFIKPLKANQQFLVYCE |
| SEQ ID NO: 48: | TEFWLGNEKIHLISTQSAIPY |
| SEQ ID NO: 49: | NANFKFTDHLKYVMLPVADQDQCIR |
| SEQ ID NO: 50: | GSEVVKRPRRYLYQWLGAPVPYPDPL |
| SEQ ID NO: 51: | PCQWWRPTTTSTRCCT |
| SEQ ID NO: 52: | PGEDFRMATLYSRTQTPRAELK |
| SEQ ID NO: 53: | DGSLWRYRAGLAASLAGP |
| SEQ ID NO: 54: | VTGVVLFRQLAPRAKLDAFFA |
| SEQ ID NO: 55: | KASYLDCIRAIAANEADAV |

In aspects, the one or more Tregitopes of the modified polypeptides of the Tregitope-blood component conjugates and the modified polypeptides used to form the Tregitope-blood component conjugates have a sequence comprising, consisting of, or consisting essentially of one or more of SEQ ID NOS: 1-55 and (and/or fragments or variants thereof), and optionally 1 to 12 additional amino acids distributed in any ratio on the N terminus and/or C-terminus of the polypeptide of SEQ ID NOS: 1-55. In aspects, the one or more Tregitopes have a core amino acid sequence comprising, consisting of, or consisting essentially of one or more peptides or polypeptides having an amino acid sequence of SEQ ID NOS: 1-55, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, one or more Tregitopes have a core sequence comprising, consisting of, or consisting essentially of one or more peptides or polypeptides having an amino acid sequence of SEQ ID NOS: 1-55 (and/or fragments and variants thereof), optionally with extensions of 1 to 12 amino acids on the C-terminal and/or the N-terminal of the core sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the Tregitope with the flanking amino acids is still able to bind to the same HLA molecule (i.e., retain MHC binding propensity) as said polypeptide core sequence without said flanking amino acids. In aspects, said Tregitope with the flanking amino acids is still able to bind to the same HLA molecule (i.e., retain MHC binding propensity) and/or retain the same TCR specificity, and/or retain regulatory T cell stimulating or suppressive activity, as said Tregitope core sequence without said flanking amino acids. In aspects, said flanking amino acid sequences are those that also flank the Tregitope included therein in the naturally occurring protein, e.g., in an IgG antibody. In aspects, the extension(s) may serve and be designed to improve the biochemical properties of the peptides or polypeptides (e.g., but not limited to, solubility or stability) or to improve the likelihood for efficient proteasomal processing of the peptide. In aspects, said flanking amino acid sequences as described herein may serve as a MHC stabilizing region. The use of a longer peptide may allow endogenous processing by patient cells and may lead to more effective antigen presentation and induction of T cell responses. In aspects, the one or more Tregitopes of the modified polypeptide may optionally have one or more linkers, which may optionally be cleavage sensitive sites, adjacent to their N-terminal and/or C-terminal end. In such a modified polypeptide, two or more of the Tregitopes may have a cleavage sensitive site between them. Alternatively, two or more of the Tregitopes may be connected directly to one another or through a linker that is not a cleavage sensitive site. Additional linkers will be described below. In aspects, the modified polypeptide comprising the one or more Tregitopes and/or the Tregitopes contained therein can be in either neutral (uncharged) or salt forms, and may be either free of or include modifications such as glycosylation, side chain oxidation, or phosphorylation. In certain aspects, the modified polypeptide comprising the one or more Tregitopes peptides or polypeptides can be capped with an N-terminal acetyl and/or C-terminal amino group. In aspects, the one or more Tregitopes included in the modified polypeptide can be capped with an N-terminal acetyl and/or C-terminal amino group.

In aspects, the instant disclosure is directed to a polypeptide comprising an amino acid sequence having at least 75%, 80%, 85%, 90%, or 95% homology to any one of SEQ ID NOS: 1-55 (and/or fragments thereof), wherein said polypeptide is still able to bind to a same HLA molecule (i.e., retain MHC binding propensity) and/or retain the same TCR specificity, and/or retain regulatory T cell stimulating or suppressive activity.

In aspects, the modified polypeptides of the Tregitope-blood component conjugates and the modified polypeptides used to form the Tregitope-blood component conjugates further comprise one or more T1Dgen peptides (e.g., PPI-derived peptides). In aspects, said one or more T1Dgen peptides may be optionally separated from one or more Tregitopes by a cleavage site (e.g., a lysosomal cleavage site). In aspects, said one or more T1Dgen peptides of the modified polypeptides comprise, consist, or consist essentially of an amino acid sequence of SEQ ID NOS: 56-63 (and/or fragments or variants thereof). The phrase “consisting essentially of” is intended to mean that a T1Dgen peptide of the modified polypeptides according to the present disclosure, in addition to the sequence according to any of SEQ ID NOS: 56-63 (or a fragment or variant thereof), contains additional amino acids or residues that may be present at either terminus of the peptide and/or on a side chain that are not necessarily forming part of the peptide that functions as an MHC ligand and provided they do not substantially impair the activity of the peptide to function as a an antigen. In aspects, the one or more T1Dgen peptides comprises, consists of, or consists essentially of the amino acid sequence of SEQ ID NO: 63. In aspects, the one or more T1Dgen peptides of the modified polypeptide may optionally have one or more linkers, which may optionally be cleavage sensitive sites, adjacent to their N-terminal and/or C-terminal end. In such a modified polypeptide, two or more of the T1Dgen peptides may have a cleavage sensitive site between them. Alternatively, two or more of the T1Dgen peptides may be connected directly to one another or through a linker that is not a cleavage sensitive site. Further, in aspects, a T1Dgen peptide and a Tregitope of the modified polypeptide may have a cleavage sensitive site between them or may be connected directly to one another or through a linker that is not a cleavage sensitive site. In aspects, the modified polypeptides of the Tregitope-blood component conjugates and the modified polypeptides used to form the Tregitope-blood component conjugates include one or more of the following T1Dgen, fragments thereof, variants thereof, and fragments of such variants:

| | |
|---|---|
| SEQ ID NO: 56: | LQPLALEGSLQKRGI |
| SEQ ID NO: 57: | SHLVEALYLVCGERG |
| SEQ ID NO: 58: | SHLVEALYLVCG |
| SEQ ID NO: 59: | LCGSHLVEALYLVCG |
| SEQ ID NO: 60: | AAAFVNQHLCGSHLV |
| SEQ ID NO: 61: | GAGSLQPLALEGSLQKRGIV |
| SEQ ID NO: 62: | GSLQPLALEGSLQKRGIV |
| SEQ ID NO: 63: | RGFFYTPKTRREAEDLQV |

In aspects, said one or more T1Dgen peptides of the modified polypeptides of the Tregitope-blood component conjugates and the modified polypeptides used to form the Tregitope-blood component conjugates comprise, consist, or consist essentially of an amino acid sequence of SEQ ID NOS: 56-63 (and/or fragments or variants thereof), and optionally 1 to 12 additional amino acids distributed in any ratio on the N-terminus and/or C-terminus of the polypeptide of SEQ ID NOS: 56-63. In aspects, said one or more T1Dgen peptides have a core amino acid sequence comprising, consisting of, or consisting essentially of one or more peptides or polypeptides having an amino acid sequence of SEQ ID NOS 56-63, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, said one or more T1Dgen peptides have a core sequence comprising, consisting of, or consisting essentially of one or more peptides or polypeptides having an amino acid sequence of SEQ ID NOS. 56-63 (and/or fragments and variants thereof), optionally with extensions of 1 to 12 amino acids on the C-terminus and/or the N-terminus, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the T1Dgen peptide with the flanking amino acids is still able to bind to an HLA molecule (i.e., retain MHC binding). In aspects, said T1Dgen peptide with the flanking amino acids is still able to bind to the same HLA molecule (i.e., retain MHC binding propensity) and retain the same TCR specificity as said T1Dgen peptide core sequence without said flanking amino acids. In aspects, said flanking amino acid sequences are those that also flank the said T1Dgen peptide included therein in the naturally occurring protein. For example, for a peptide or polypeptide have a core sequence comprising, consisting of, or consisting essentially of one or more peptides or polypeptides having an amino acid sequence of SEQ ID NOS: 56-63 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 56-63 in the amino acid sequence of preproinsulin. In aspects, the extension(s) may serve and be designed to improve the biochemical properties of the peptides or polypeptides (e.g., but not limited to, solubility or stability) or to improve the likelihood for efficient proteasomal processing of the peptide. In aspects, said flanking amino acid sequences as described herein may serve as a MHC stabilizing region. The use of a longer peptide may allow endogenous processing by patient cells and may lead to more effective antigen presentation and induction of T cell responses. In aspects, the one or more T1Dgen peptides can be in either neutral (uncharged) or salt forms, and may be either free of or include modifications such as glycosylation, side chain oxidation, or phosphorylation.

The manner of producing the modified polypeptides of the Tregitope-blood component conjugates and the modified polypeptides used to form the Tregitope-blood component conjugates of the present disclosure will vary widely, depending upon the nature of the various elements comprising the molecule. The synthetic procedures may be selected so as to be simple, provide for high yields, and allow for a highly purified stable product. Normally, the reactive moiety will be created as the last stage, for example, with a carboxyl group, esterification to form an active ester will be the last step of the synthesis.

In aspects, the present disclosure is also directed to a method of synthesizing the modified polypeptides of the Tregitope-blood component conjugates and the modified polypeptides used to form the Tregitope-blood component conjugates, as disclosed in U.S. Pat. Nos. 6,849,714, 6,887,470, 7,256,253, and 7,307,148. In aspects, the method comprises the following steps. In the first step, the one or more Tregitope sequences of the polypeptide can be synthesized as essentially disclosed herein; and optionally the polypeptide sequence additionally comprises one or more T1Dgen sequences as disclosed herein. In the second step, if the polypeptide does not contain a cysteine, then the polypeptide may be synthesized from the carboxy terminal amino acid and the reactive moiety is added to the carboxy terminal amino acid. Alternatively, a terminal lysine (or one or more lysines) may added to the carboxy terminal amino acid and the reactive moiety is added to the terminal lysine. In the third step, if the polypeptide contains only one cysteine, then the cysteine is reacted with a protective group prior to addition of the reactive moiety to an amino acid in a less therapeutically active region of the polypeptide. In the fourth step, if the polypeptide contains two cysteines as a disulfide bridge, then the two cysteines are oxidized and the reactive moiety is added to the amino terminal amino acid, or to the carboxy terminal amino acid, or to an amino acid positioned between the carboxy terminal amino acid and the amino terminal amino acid of the polypeptide. In the fifth step, if the polypeptide contains more than two cysteines as disulfide bridges, the cysteines are sequentially oxidized in the disulfide bridges and the peptide is purified prior to the addition of the reactive moieties to the carboxy terminal amino acid.

In aspects, the present disclosure is also directed to a method of synthesizing the Tregitope-blood component conjugate. In a first step, reactive maleimidopropionamido (MPA) is added via an N-terminal lysine on the polypeptide comprising one or more Tregitopes to create a modified polypeptide. In aspects, one or more lysines are present on the N-terminus of the polypeptide, optionally present at the N-terminus of a Tregitope sequence selected from the group of SEQ. ID NOS: 1-55 as disclosed herein. Optionally, polyethylene glycol linker, such as PEG2 or PEG12, is present between the one or more lysines and a Tregitope sequence, or at the N-terminus of a Tregitope sequence. In aspects, a lysosomal cleavage site, such as a Cathepsin B site, optionally consisting (sequentially from N-terminus to C-terminus) of valine and citrulline, is present between the PEG2 or PEG12 moiety and the Tregitope sequence. The lysosomal cleavage site (such as Cathepsin B site) may be incorporated to provide a lysosomal protease site, allowing the Tregitope to be released into the lysosomal compartment. In aspects, lysosomal cleavage site (such as Cathepsin B site) is present to provide a lysosomal protease site, allowing the Tregitope to be released into cells, preferably into the early endosome. In a preferred embodiment, the lysosomal cleavage site (such as Cathepsin B site) is present to provide a lysosomal protease site, allowing the Tregitope to be released into cells, such as into a membrane-enclosed vesicle (such as the early endosome, late endosome, or lysosome), such that the Tregitope may be processed for antigen presentation. In aspects, the Tregitope is presented as antigen by immune cells, such as macrophages or antigen-presenting cells, preferably presented as an MHC class II antigen. In aspects, a lysosomal cleavage site, such as a Cathepsin B site, optionally consisting (sequentially from N-terminus to C-terminus) of valine and citrulline, is present between the PEG2 or PEG12 moiety and the Tregitope sequence, and/or between one or more Tregitopes. In aspects, one or more Tregitopes may be present on the construct, optionally more proximate to the C-terminus than the linker. In aspects, one or more lysosomal cleavage sites are present between multiple Tregitopes (for example, such that a single lysosomal cleavage site separates two Tregitopes, or such that one lysosomal cleavage site is present between a first and second Tregitope, and another lysosomal cleavage site is present between a second and third Tregitope, and so on). In aspects, one or more T1Dgen peptides (e.g., PPI-derived peptides) as disclosed herein may be present on the construct, optionally separated from one or more Tregitopes by a lysosomal cleavage site. In aspects, one or more lysosomal cleavages sites are present between multiple T1Dgen peptides (for example, such that a single lysosomal cleavage site separates two T1Dgen peptides, or such that one lysosomal cleavage site is present between a first and second T1Dgen peptides, and another lysosomal cleavage site is present between a second and third T1Dgen peptides, and so on). In aspects, a norleucine (Nle) residue is present at the C-terminus as a means to quantitate the amount of Tregitope peptide incorporated into the final Tregitope-blood component conjugate, for example for evaluation by mass spectrometry. In aspects, the C-terminus of the polypeptide is capped with a C-terminal amino group. In a second step, a maleimide-based chemistry is used to covalently link the modified polypeptide to a blood component, preferably serum albumin, in a 1:1 molar ratio. The second step may be performed in vivo or ex vivo, as described further below and in the examples of the present disclosure.

In aspects, the formation of the Tregitope-blood component conjugate protects the Tregitope, when present in vivo, from rapid degradation by peptidases, rapid clearance from circulation, and/or rapid kidney excretion. In aspects, the formation of the Tregitope-blood component conjugate significantly extends the half-life of the Tregitope in vivo. In aspects, the formation of the Tregitope-blood component conjugate allows wide distribution of the Tregitope-blood component conjugate throughout the body of a subject. In aspects, the Tregitope-blood component conjugate does not cross the blood-brain barrier when present in the plasma of a subject. In aspects, the Tregitope-blood component conjugate aid in delivery of Tregitopes to appropriate immune cells, such as macrophages and/or antigen-presenting cells (APCs). In aspects, upon delivery of Tregitopes to appropriate immune cells, such as macrophages and/or APCs, the Tregitopes are encompassed in a membrane-bound vesicle, preferably a vesicle in the endocytic pathway such as an early endosome, late endosome, or lysosome. In aspects, the Tregitopes, once processed by the appropriate immune cells, such as macrophages and/or APCs, are presented as MHC class II antigens.

In aspects, the Tregitope in the Tregitope-blood component conjugate has a plasma half-life in vivo of up to 12 hours. In aspects, the Tregitope in the Tregitope-blood component conjugate has a plasma half-life in vivo of up to 1 day. In aspects, the Tregitope in the Tregitope-blood component conjugate has a plasma half-life in vivo of up to 40-48 hours. In aspects, the Tregitope in the Tregitope-blood component conjugate has a plasma half-life in vivo of up to 60 hours. In aspects, the Tregitope in the Tregitope-blood component conjugate has a plasma half-life in vivo of up to 15 days.

In aspects, the modified polypeptide comprising one or more Tregitopes is administered to a subject, wherein upon administration, the modified polypeptide reacts in vivo with a reactive functionality of a circulating blood component. In aspects, the peptide is administered to a human subject, and the blood component is human albumin, preferably the circulating albumin of the human subject.

In aspects, the modified polypeptides used to form the Tregitope-blood component conjugates is capable of forming a bond ex vivo with a reactive functionality on a blood component, wherein upon formation of a bond between the reactive moiety of the modified polypeptide and the reactive functionality on the blood component, a Tregitope-blood component conjugate is formed, as disclosed in U.S. Pat. Nos. 6,849,714, 6,887,470, 7,256,253, and 7,307,148. In aspects, the modified polypeptide as disclosed herein is configured to covalently attach to a reactive functionality of a blood component outside of the body. In aspects, the blood component is albumin. In aspects, the blood component is selected from the group of recombinant albumin, human recombinant albumin, and albumin from a genomic source.

In aspects, the present disclosure is also directed to an ex vivo method of synthesizing the modified Tregitope peptide and the Tregitope-blood component conjugate, as disclosed in U.S. Pat. Nos. 6,849,714, 6,887,470, 7,256,253, and 7,307,148. In aspects, the modified polypeptide as disclosed herein is added to blood, serum or saline solution containing human serum albumin to permit covalent bond formation between the modified therapeutic peptide and the blood component. In aspects, the polypeptide comprising one or more Tregitopes as disclosed herein is modified with maleimide and it is reacted with serum albumin in saline solution. In aspects, once the modified polypeptide has reacted with the blood component, to form a Tregitope-blood component conjugate, the conjugate may be administered to the subject. In aspects, after the modified polypeptide has reacted with the blood component to form the conjugate, but before the conjugate is administered to the subject, the conjugate may be separated from non-conjugated blood components in the reaction mixture. In aspects, conjugate may be separated from non-conjugated blood components in the reaction mixture by separating substances on the basis of their varying strengths of hydrophobic interactions with hydrophobic ligands immobilized to an uncharged matrix. In aspects, the uncharged matrix may be a hydrophobic solid support, wherein the support comprises a column containing a hydrophobic resin such as, but not limited to, octyl sepharose, phenyl sepharose and butyl sepharose. In aspects, this technique may be performed with moderately high concentrations of salts (˜1M) in the start buffer (salt promoted adsorption). Elution is achieved by a linear or stepwise decrease in salt concentration. The type of ligand, the degree of substitution, the pH and the type and concentration of salt used during the adsorption stage have a profound effect on the overall performance (e.g. selectivity and capacity) of a HIC matrix (Hydrophobic Interaction Chromatography matrix).

The solvent is one of the most important parameters that influence capacity and selectivity in HIC (Hydrophobic Interaction Chromatography). In general, the adsorption process is more selective than the desorption process. It is therefore important to optimize the start buffer with respect to pH, type of solvent, type of salt, and concentration of salt. The addition of various “salting-out” salts to the sample promotes ligand-protein interactions in HIC. As the concentration of salt is increased, the amount of bound protein increases up to the precipitation point for the protein. Each type of salt differs in its ability to promote hydrophobic interactions.

Increasing the salting-out effect strengthens the hydrophobic interactions, whereas increasing the chaotropic effect weakens them. Examples of salts with high salting-out effects, in order from greater salting-out effect to smaller salting-out effect, include: PO₄³⁻, SO₄²⁻, CH₃COO⁻, Cl⁻, Br⁻, NO₃⁻, ClO₄⁻, I⁻, and SCN⁻. Examples of salts with high chaotropic effects, in order from greater chaotropic effect to smaller chaotropic effect, include: NH₄⁺, Rb⁺, K⁺, Na⁺, Cs⁺, Li⁺, Mg₂⁺, and Ba²⁺. The most commonly used salts for HIC are ammonium sulfate ((NH₄)₂SO₄), sodium sulfate ((Na)₂SO₄)), magnesium sulfate (MgSO₄), sodium chloride (NaCl), potassium chloride (KCl), and ammonium acetate (CH₃COONH₄).

Protein binding to HIC adsorbents is promoted by moderate to high concentrations of “salting-out” salts, most of which also have a stabilizing influence on protein structure due to their preferential exclusion from native globular proteins, i.e., the interaction between the salt and the protein surface is thermodynamically unfavorable. The salt concentration should be high enough (e.g. 500-1000 mM) to promote ligand-protein interactions yet below that which causes precipitation of the protein in the sample. In the case of albumin, the salt concentration should be kept below 3M (moles per liter). The principle mechanism of salting-out consists of the salt-induced increase of the surface tension of water (Melander and Horvath, 1977). Thus, a compact structure becomes energetically more favorable because it corresponds to smaller protein-solution interfacial area. Under these conditions, for example buffer composed of SO₄²⁻, PO₄²⁻ or CH₃COO⁻ with any counter ion, these salts exhibit their salting-out effect upon essentially all conjugated albumin described herein in a manner different to non-conjugated albumin (i.e. mercaptalbumin and albumin capped with cysteine), thus enabling a consistent chromatographic separation between conjugated albumin versus non-conjugated albumin. Thus, lower concentrations of salt are required to promote interactions between ligand and conjugated albumin than between ligand and non-conjugated albumin. This chromatographic separation is essentially independent of (a) the sequence of albumin (e.g. human, mouse, rat, etc.) (b) the source of albumin (i.e. plasma derived or recombinant) (c) the molecular weight of the conjugated modified Tregitope, (d) the position of the reactive moiety within the structure of the molecule, (e) the peptide sequence or chemical structure of the molecule, and (f) the three-dimensional structure of the conjugated molecule, e.g., linear versus loop structure.

In aspects, the salt of the aqueous buffer has a sufficient salting-out effect. In aspects, for providing a sufficient salting out effect, the salt may be phosphate, sulfate and acetate. In aspects, the selection of the cation of the buffer is can be selected, without limitation, from the group consisting of NH₄⁺, Rb⁺, K⁺, Na⁺, Cs⁺, Li⁺, Mg²⁺ and Ba²⁺. In aspects, the aqueous buffer may be selected from the group of ammonium phosphate, ammonium sulfate and magnesium phosphate. In aspects, the buffer pH is between 3.0 and 9.0; more preferably between 6.0 and 8.0, and even more preferably, the pH is 7.0. In aspects, the buffer and the hydrophobic solid support are at room temperature (about 25° C.) or at 4° C. or in between.

In aspects, the Tregitope-blood component conjugates and modified polypeptides comprising Tregitopes, with said modified peptides being capable of reacting with blood components to form such Tregitope-blood component conjugates, of the present disclosure can be purified to homogeneity or partially purified. It is understood, however, that preparations in which such compositions are not purified to homogeneity are useful. The critical feature is that the preparation allows for the desired function of the Tregitope and the optional T1Dgen, even in the presence of considerable amounts of other components. Thus, the present disclosure encompasses various degrees of purity. In one embodiment, the language “substantially free of cellular material” includes preparations of the Tregitope-blood component conjugates and modified polypeptides comprising Tregitopes having less than about 30% (by dry weight) other proteins (e.g., contaminating protein; “other proteins” do not include T1Dgen peptides or carrier proteins, such as albumin), less than about 20% other proteins, less than about 10% other proteins, less than about 5% other proteins, less than about 4% other proteins, less than about 3% other proteins, less than about 2% other proteins, less than about 1% other proteins, or any value or range therebetween.

In aspects, when a Tregitope-blood component conjugates or a modified polypeptide comprising Tregitopes of the present disclosure is recombinantly produced, said Tregitope composition can also be substantially free of culture medium, for example, culture medium represents less than about 20%, less than about 10%, or less than about 5% of the volume of the Tregitope, T1Dgen, carrier protein, nucleic acid, or chimeric or fusion polypeptide preparation. The language “substantially free of chemical precursors or other chemicals” includes preparations of the polypeptide, nucleic acid, or chimeric or fusion polypeptide in which it is separated from chemical precursors or other chemicals that are involved in the Tregitope composition's synthesis. The language “substantially free of chemical precursors or other chemicals” can include, for example, preparations of the Tregitope, T1Dgen, carrier protein, nucleic acid, or chimeric polypeptides having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 1% chemical precursors or other chemicals.

As used herein, two polypeptides (or a region of the polypeptides) are substantially homologous or identical when the amino acid sequences are at least about 45-55%, typically at least about 70-75%, more typically at least about 80-85%, more typically greater than about 90%, and more typically greater than 95% or more homologous or identical. To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As is known in the art, the percent identity between the two sequences is a function of the number of identical positions shared by the sequences, taking into account the number of gaps, and the length of each gap, which need to be introduced for optimal alignment of the two sequences. Sequence homology for polypeptides is typically measured using sequence analysis software. As used herein, amino acid or nucleic acid “homology” is equivalent to amino acid or nucleic acid “identity”. In aspects, the percent homology between the two sequences is a function of the number of identical positions shared by the sequences (e.g., percent homology equals the number of identical positions/total number of positions ×100).

In aspects, the present disclosure also encompasses polypeptides (e.g., Tregitopes and T1Dgens as disclosed herein) having a lower degree of identity but having sufficient similarity so as to perform one or more of the same functions performed by a polypeptide of the present disclosure, particularly that any such variants retain MHC binding propensity and/or TCR specificity. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found (Bowie J U et al., (1990), Science, 247(4948):130610, which is herein incorporated by reference in its entirety).

In aspects, a variant polypeptide (e.g., a variant Tregitope or T1Dgen) can differ in amino acid sequence by one or more substitutions, deletions, insertions, inversions, fusions, and truncations or a combination of any of these. Variant polypeptides can be fully functional (e.g., retain MHC binding propensity and/or TCR specificity, and in aspects in which the variant polypeptide is a Tregitope, and/or retain regulatory T cell stimulating or suppressive activity) or can lack function in one or more activities. Fully functional variants typically contain only conservative variation or variation in non-critical residues or in non-critical regions; in this case, typically MHC contact residues provided MHC binding is preserved. Functional variants can also contain substitution of similar amino acids that result in no change or an insignificant change in function (e.g., retain MHC binding propensity and/or TCR specificity, and in aspects in which the variant polypeptide is a Tregitope, and/or retain regulatory T cell stimulating or suppressive activity). Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues. In aspects, a variant and/or a homologous Tregitope polypeptide retains the desired regulatory T cell stimulating or suppressive activity of the instant disclosure. Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues. In aspects, functional variants of a polypeptide having a sequence (or a core sequence) comprising, consisting of, or consisting essentially of one or more of SEQ ID NOS: 1-55 as disclosed herein may contain one or more conservative substitutions, and in aspects one or more non-conservative substitutions, at amino acid residues which are not believed to be essential for functioning (with amino acid residues considered being essential for functioning, including, e.g., retain MHC binding propensity and/or TCR specificity, and/or retain regulatory T cell stimulating or suppressive activity) of the instantly-disclosed polypeptides. For example, in aspects, a variant polypeptide having a sequence (or a core sequence) comprising, consisting of, or consisting essentially of one or more of SEQ ID NOS: 1-55, or fragments thereof as disclosed herein, may contain one or more conservative substitutions (and in aspects, a nonconservative substitution) in one or more HLA contact residues, provided HLA binding is preserved. MHC binding assays are well known in the art. In aspects, such assays may include the testing of binding affinity with respect to MHC class I and class II alleles in in vitro binding assays, with such binding assays as are known in the art. Examples include, e.g., the soluble binding assays as disclosed in U.S. Pat. No. 7,884,184 or PCT/US2020/020089, both of which are herein incorporated by reference in their entireties. Additionally, in aspects, a fully functional variant polypeptide having a sequence (or a core sequence) comprising, consisting of, or consisting essentially of one or more of SEQ ID NOS: 1-55 as disclosed herein do not contain mutations at one or more critical residues or regions, such as TCR contact residues.

In aspects, the TCR-binding epitope (which can be referred to as TCR binding residues, TCR facing epitope, TCR facing residues, or TCR contacts) for a 9-mer identified epitope (which may be a 9-mer fragment of one or more of SEQ ID NOS: 1-55 as disclosed herein or a 9-mer fragment of a concatemeric peptide as disclosed herein) that bind to a MHC class II molecule are at position 2, 3, 5, 7, and 8 of the identified epitope, while the MHC-binding agretope (which can be referred to as MHC contacts, MHC facing residues, MHC-binding residues, or MHC-binding face) for a 9-mer identified epitope (which may be a 9-mer fragment of one or more of SEQ ID NOS: 1-55 as disclosed herein or a 9-mer fragment of a concatemeric peptide as disclosed herein) that bind to a MHC class II molecule are at position 1, 4, 6, and 9, both as counted from the amino terminal.

In aspects, the TCR binding epitope for a 9-mer identified epitope (which may be a 9-mer fragment of one or more of SEQ ID NOS: 1-55 or as disclosed herein or a 9-mer fragment of a concatemeric peptide) that binds to a MHC class I molecule are at position 4, 5, 6, 7, and 8 of the identified epitope, while the MHC binding agretope for a 9-mer identified epitope (which may be a 9-mer fragment of one or more of SEQ ID NOS: 1-55 as disclosed herein or a 9-mer fragment of a concatemeric peptide as disclosed herein) that bind to a MHC class I molecule are at position 1, 2, 3, and 9, both as counted from the amino terminal.

In aspects, the TCR binding epitope for a 10-mer identified epitope that bind to a MHC class I molecule are at position 4, 5, 6, 7, 8, and 9 of the identified epitope (which may be a 10-mer fragment of one or more of SEQ ID NOS: 1-55 as disclosed herein, or a 10-mer fragment of a concatemeric peptide as disclosed herein, or a 10-mer peptide containing a 9-mer of one or more of SEQ ID NOS: 1-55), while the MHC binding agretope for a 10-mer identified epitope (which may be a 10-mer fragment of one or more of SEQ ID NOS: 1-124 as disclosed herein or a 10-mer fragment of a concatemeric peptide as disclosed herein, or a 10-mer peptide containing a 9-mer of one or more of SEQ ID NOS: 1-55) that bind to a MHC class I molecule are at position 1, 2, 3, 9, and 10, both as counted from the amino terminal.

In aspects, the TCR-binding epitope for a 9-mer identified epitope (which may be a 9-mer fragment of one or more of SEQ ID NOS: 1-55 as disclosed herein or a 9-mer fragment of a concatemeric peptide as disclosed herein) that bind to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) of the identified epitope, while the MHC binding agretope for a 9-mer identified epitope (which may be a 9-mer fragment of one or more of SEQ ID NOS: 1-55 as disclosed herein or a 9-mer fragment of a concatemeric peptide as disclosed herein) is the complementary face to the TCR facing residues, both as counted from the amino terminal.

In aspects, the TCR binding epitope for 9-mer identified epitope (which may be a 9-mer fragment of one or more of SEQ ID NOS: 1-55 as disclosed herein or a 9-mer fragment of a concatemeric peptide as disclosed herein) that bind to a MHC class I molecule are at positions 4, 5, 6, 7, and 8; 1, 4, 5, 6, 7 and 8; or 1, 3, 4, 5, 6, 7, and 8 of the identified epitope, while the MHC binding agretope for a 9-mer identified epitope (which may be a 9-mer fragment of one or more of SEQ ID NOS: 1-55 as disclosed herein or a 9-mer fragment of a concatemeric peptide as disclosed herein) is the complementary face to the TCR facing residues, both as counted from the amino terminal.

In aspects, the TCR-binding epitope for a 10-mer identified epitope (which may be a 10-mer fragment of one or more of SEQ ID NOS: 1-55 as disclosed herein, or a 10-mer fragment of a concatemeric peptide as disclosed herein, or a 10-mer peptide containing a 9-mer of one or more of SEQ ID NOS: 1-55) that bind to a MHC class I molecule are at any combination of residues at positions 1, 3, 4, 5, 6, 7, 8, and 9 of the identified epitope, while the MHC binding agretope for a 10-mer identified epitope (which may be a 10-mer fragment of one or more of SEQ ID NOS: 1-55 as disclosed herein or a 10-mer fragment of a concatemeric peptide as disclosed herein, or a 10-mer peptide containing a 9-mer of one or more of SEQ ID NOS: 1-55) is the complementary face to the TCR facing residues, both as counted from the amino terminal.

Based on the above, it should be understood that in aspects in which one or more 9-mers and/or 10-mer epitopes are contained within a longer polypeptide and are predicted to bind one or more Class I or Class II MHC molecules and are occurring in close proximity to each other in a naturally occurring sequence (e.g., wherein position 1 of each pair of binding 9-mers and/or 10-mers fall within, e.g., 3 amino acids of each other), such epitopes may be combined to form an epitope cluster. In a given cluster, any given amino acid may be, with respect to a given 9-mer epitope or 10-mer epitope, MHC facing and, with respect to another 9-mer epitope, TCR facing.

In aspects, the present disclosure also includes Tregitope-blood component conjugates and modified polypeptides comprising Tregitopes that include polypeptide fragments of the instantly disclosed Tregitopes and T1Dgens. In aspects, the present disclosure also encompasses fragments of the variants of the Tregitopes and T1Dgens described herein. In aspects, as used herein, a fragment comprises at least about nine contiguous amino acids. Useful fragments (and fragments of the variants of the polypeptides and concatemeric polypeptides described herein) include those that retain one or more of the biological activities, particularly: MHC binding propensity and/or TCR specificity, and/or retain regulatory T cell stimulating or suppressive activity. Biologically active fragments are, for example, about 9, 10, 11, 12, 1, 14, 15, 16, 20, 25, 30, 40, 50, 60, 70, 80, 90, 100 or more amino acids in length, including any value or range therebetween. Fragments can be discrete (not fused to other amino acids or polypeptides) or can be within a larger polypeptide. Several fragments can be comprised within a single larger polypeptide. In aspects, a fragment designed for expression in a host can have heterologous pre- and pro-polypeptide regions fused to the amino terminus of the polypeptide fragment and an additional region fused to the carboxyl terminus of the fragment.

In aspects, Tregitopes and/or T1Dgens of the present disclosure can include allelic or sequence variants (“mutants”) or analogs thereof, or can include chemical modifications (e.g., pegylation, glycosylation). In aspects, a mutant retains the same functions performed by a polypeptide encoded by a nucleic acid molecule of the present disclosure, particularly MHC binding propensity and/or TCR specificity, and in aspects in which the variant polypeptide is a Tregitope, and/or retain regulatory T cell stimulating or suppressive activity. In aspects, a mutant can provide for enhanced binding to MHC molecules. In aspects, a mutant can lead to enhanced binding to TCRs. In another instance, a mutant can lead to a decrease in binding to MHC molecules and/or TCRs. Also contemplated is a mutant that binds, but does not allow signaling via the TCR.

The manner of producing the polypeptides of the present disclosure will vary widely, depending upon the nature of the various elements comprising the molecule. For example, an isolated polypeptide can be purified from cells that naturally express it, purified from cells that have been altered to express it (recombinant), or synthesized using known protein synthesis methods. The synthetic procedures may be selected so as to be simple, provide for high yields, and allow for a highly purified stable product. For example, polypeptides of the instant disclosure can be produced either from a nucleic acid disclosed herein, or by the use of standard molecular biology techniques, such as recombinant techniques, mutagenesis, or other known means in the art. An isolated polypeptide can be purified from cells that naturally express it, purified from cells that have been altered to express it (recombinant), or synthesized using known protein synthesis techniques. In aspects, a polypeptide of the instant disclosure is produced by recombinant DNA or RNA techniques. In aspects, a polypeptide of the instant disclosure can be produced by expression of a recombinant nucleic acid of the instant disclosure in an appropriate host cell. For example, a nucleic acid molecule encoding the polypeptide is cloned into an expression cassette or expression vector, the expression cassette or expression vector introduced into a host cell and the polypeptide expressed in the host cell. The polypeptide can then be isolated from the cells by an appropriate purification scheme using standard protein purification techniques. Alternatively a polypeptide can be produced by a combination of ex vivo procedures, such as protease digestion and purification. Further, polypeptides of the instant disclosure can be produced using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, the polypeptides can be purified to homogeneity or partially purified. It is understood, however, that preparations in which the polypeptides are not purified to homogeneity are useful. The critical feature is that the preparation allows for the desired function of the composition, even in the presence of considerable amounts of other components. Thus, the present disclosure encompasses various degrees of purity. In one embodiment, the language “substantially free of cellular material” includes preparations of the polypeptides, concatemeric polypeptides, and chimeric or fusion polypeptides having less than about 30% (by dry weight) other proteins (e.g., contaminating protein), less than about 20% other proteins, less than about 10% other proteins, less than about 5% other proteins, less than about 4% other proteins, less than about 3% other proteins, less than about 2% other proteins, less than about 1% other proteins, or any value or range therebetween.

In aspects, when a polypeptide of the present disclosure is recombinantly produced, the composition can also be substantially free of culture medium, for example, culture medium represents less than about 20%, less than about 10%, or less than about 5% of the volume of the polypeptide preparation. The language “substantially free of chemical precursors or other chemicals” includes preparations of the polypeptides in which it is separated from chemical precursors or other chemicals that are involved in the polypeptides synthesis. The language “substantially free of chemical precursors or other chemicals” can include, for example, preparations of the polypeptides having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 10% chemical precursors or other chemicals.

In aspects, the present disclosure also includes pharmaceutically acceptable salts of the polypeptides disclosed herein. “Pharmaceutically acceptable salt” means a salt that is pharmaceutically acceptable and that possesses the desired pharmacological activity of the parent peptide or polypeptide. As used herein, “pharmaceutically acceptable salt” refers to derivative of the instantly-disclosed polypeptides, wherein such compounds are modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In aspects, the Tregitope-blood component conjugates and/or the modified polypeptides used to form the Tregitope-blood component conjugates of the present disclosure are combined in admixture with an antigen and/or a therapeutic protein. Such compositions are useful in methods of inducing tolerance to the antigen or a therapeutic protein in a subject in need thereof, wherein local delivery of the admixture with an antigen or a therapeutic protein results in increased tolerance to the antigen in the subject, and delivered with an appropriate excipient resulting in induced tolerance to the antigen or a therapeutic protein. This combination may be administered with the Tregitope-blood component conjugates and/or the modified polypeptides used to form the Tregitope-blood component conjugates of the present disclosure bound either covalently or non-covalently, or they may be administered as an admixture, or a branched or chemically-link preparation. Such compositions are useful in methods of inducing tolerance to an antigen, an allergen, and/or a therapeutic protein (e.g., but not limited to, Insulin and/or preproinsulin). For example, such composition are useful in a subject in need thereof, wherein local delivery of the admixture with an antigen and/or therapeutic protein results in increased tolerance to the antigen or therapeutic protein in the subject, and delivered with an appropriate excipient resulting in induced tolerance to the antigen or therapeutic protein. This combination may be administered with the Tregitope compositions of the present disclosure bound either covalently or non-covalently, or they may be administered as an admixture.

### Nucleic Acids

In aspects, the present disclosure is directed to a nucleic acid (e.g., DNAs (including cDNA, RNAs (such as, but limited to mRNA)), vectors, viruses, or hybrids thereof, all of which may be isolated, synthetic, or recombinant) encoding in whole or in part the instantly-disclosed Tregitope-blood component conjugates and/or the modified polypeptides used to form the Tregitope-blood component conjugates. In aspects, the nucleic acid further comprises, or is contained within, an expression cassette, a plasmid, and expression vector, or recombinant virus, wherein optionally the nucleic acid, or the expression cassette, plasmid, expression vector, or recombinant virus is contained within a cell, optionally a human cell or a non-human cell, and optionally the cell is transformed with the nucleic acid, or the expression cassette, plasmid, expression vector, or recombinant virus. In aspects, cells are transduced, transfected, or otherwise engineered to contain within one or more of e.g., Tregitope-blood component conjugates and/or the modified polypeptides used to form the Tregitope-blood component conjugates of the present disclosure; isolated, synthetic, or recombinant nucleic acids, expression cassettes, plasmids, expression vectors, or recombinant viruses as disclosed herein; and/or isolated, synthetic, or recombinant chimeric or fusion polypeptide compositions as disclosed herein. In aspects, the cell can be a mammalian cell, bacterial cell, insect cell, or yeast cell. In aspects, the nucleic acid molecules of the present disclosure can be inserted into vectors and used, for example, as expression vectors or gene therapy vectors. Gene therapy vectors can be delivered to a subject by, e.g., intravenous injection, local administration (U.S. Pat. No. 5,328,470) or by stereotactic injection (Chen S H et al., (1994), Proc Natl Acad Sci USA, 91(8):3054-7, which are herein incorporated by reference in their entirety). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. Such pharmaceutical compositions can be included in a container, pack, or dispenser together with instructions for administration. In aspects, the present disclosure is directed to a cell comprising a vector of the present disclosure. In aspects, the cell can be a mammalian cell, bacterial cell, insect cell, or yeast cell.

The nucleic acid of the instant disclosure may be DNAs (including but not limited to cDNA) or RNAs (including but not limited to mRNA), single- or double-stranded. The nucleic acid may be, e.g., DNA, cDNA, PNA, CNA, RNA, either single- and/or double-stranded, or native or stabilized forms of polynucleotides as are known in the art. The nucleic acid is typically DNA or RNA (including mRNA). The nucleic acid may be produced by techniques well known in the art, such as synthesis, or cloning, or amplification of the sequence encoding the immunogenic polypeptide; synthesis, or cloning, or amplification of the sequence encoding the cell membrane addressing sequence; ligation of the sequences and their cloning/amplification in appropriate vectors and cells. The nucleic acids provided herein (whether RNA, DNA, vectors, viruses or hybrids thereof) that encode in whole or in part Tregitope-blood component conjugates and/or the modified polypeptides used to form the Tregitope-blood component conjugates of the present disclosure can be isolated from a variety of sources, genetically engineered, amplified, synthetically produced, and/or expressed/generated recombinantly. Recombinant polypeptides generated from these nucleic acids can be individually isolated or cloned and tested for a desired activity. Any recombinant expression system can be used, including e.g. in vitro, bacterial, fungal, mammalian, yeast, insect or plant cell expression systems. In aspects nucleic acids provided herein are synthesized in vitro by well-known chemical synthesis techniques (as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066, all of which are herein incorporated by reference in their entirety). Further, techniques for the manipulation of nucleic acids provided herein, such as, e.g., subcloning, labeling probes (e.g., random-primer labeling using Klenow polymerase, nick translation, amplification), sequencing, hybridization, and the like are well-described in the scientific and patent literature (see, e.g., Sambrook, ed., Molecular Cloning: A Laboratory Manual (2^{ND} Ed.), Vols. 1-3, Cold Spring Harbor Laboratory, (1989); Current Protocols In Molecular Biology, Ausubel, ed. John Wiley & Sons, Inc., New York (1997); Laboratory Techniques In Biochemistry And Molecular Biology: Hybridization With Nucleic Acid Probes, Part I. Theory and Nucleic Acid Preparation, Tijssen, ed. Elsevier, N.Y. (1993), all of which are herein incorporated by reference in their entirety).

As previously mentioned, the nucleic acid molecules according to the present disclosure may be provided in the form of a nucleic acid molecule per se such as naked nucleic acid molecules; a plasmid, a vector; virus or host cell, etc., either from prokaryotic or eukaryotic origin. Vectors include expression vectors that contain a nucleic acid molecule of the invention. An expression vector capable of expressing a polypeptide can be prepared. Expression vectors for different cell types are well known in the art and can be selected without undue experimentation. Generally, the (e.g., cDNA, or RNA, including mRNA) is inserted into an expression vector, such as a plasmid, in proper orientation and correct reading frame for expression. If necessary, the DNA (e.g., cDNA, or RNA, including mRNA) may be linked to the appropriate transcriptional and translational regulatory control nucleotide sequences recognized by the desired host (e.g., bacteria), although such controls are generally available in the expression vector. The vector is then introduced into the host bacteria for cloning using standard techniques. The vectors of the present invention may, for example, comprise a transcriptional promoter, and/or a transcriptional terminator, wherein the promoter is operably linked with the nucleic acid molecule, and wherein the nucleic acid molecule is operably linked with the transcription terminator. One or more peptides or polypeptides of the present disclosure may be encoded by a single expression vector. Such nucleic acid molecules may act as vehicles for delivering peptides/polypeptides to the subject in need thereof, in vivo, in the form of, e.g., DNA/RNA vaccines.

In aspects, the vector may be a viral vector comprising a nucleic acid as defined above. The viral vector may be derived from different types of viruses, such as, Swinepox, Fowlpox, Pseudorabies, Aujezky's virus, salmonella, vaccinia virus, BHV (Bovine Herpes Virus), HVT (Herpes Virus of Turkey), adenovirus, TGEV (Transmissible Gastroenteritidis Coronavirus), Erythrovirus, and SIV (Simian Immunodeficiency Virus). Other expression systems and vectors may be used as well, such as plasmids that replicate and/or integrate in yeast cells.

The instant disclosure also relates to a method for preparing a the instantly-disclosed Tregitope-blood component conjugates and/or the modified polypeptides used to form the Tregitope-blood component conjugates of the instant disclosure, the method comprising culturing a host cell containing a nucleic acid or vector as defined above under conditions suitable for expression of the nucleic acid and recovering the Tregitope-blood component conjugates and/or the modified polypeptides used to form the Tregitope-blood component conjugates. As indicated above, the proteins and peptides may be purified according to techniques known per se in the art.

### Pharmaceutical Compositions and Formulations

In aspects, the Tregitope-blood component conjugates and/or the modified polypeptides used to form the Tregitope-blood component conjugates of the present disclosure (as well as nucleic acids as disclosed herein, including nucleic acids encoding such Tregitope-blood component conjugates and/or the modified polypeptides used to form the Tregitope-blood component conjugates, and/or expression cassettes, plasmids, expression vectors, recombinant viruses, cells as disclosed herein) may be comprised in a pharmaceutical composition or formulation. In aspects, pharmaceutical compositions or formulations generally comprise a Tregitope-blood component conjugates and/or the modified polypeptides used to form the Tregitope-blood component conjugates of the present disclosure and a pharmaceutically acceptable carrier, excipient, and/or adjuvant. In aspects, the instantly-disclosed pharmaceutical compositions or formulations may further comprise diluents, adjuvants, freeze drying stabilizers, wetting or emulsifying agents, pH buffering agents, gelling or viscosity enhancing additives, and preservatives, depending on the route of administration. In aspects, said pharmaceutical compositions are suitable for administration. Pharmaceutically acceptable carriers and/or excipients are determined in part by the particular composition being administered, as well as by the particular method used to administer the composition. Accordingly, there is a wide variety of suitable formulations of pharmaceutical compositions for administering the instantly-disclosed Tregitope compositions (see, e.g., Remington's Pharmaceutical Sciences, (18^{TH} Ed, 1990), Mack Publishing Co., Easton, Pa. Publ, which is herein incorporated by reference in its entirety). In aspects, the pharmaceutical compositions are generally formulated as sterile, substantially isotonic, and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, excipients, and reagents, are used interchangeably and represent that the materials are capable of administration to or upon a subject without the production of undesirable physiological effects to a degree that would prohibit administration of the composition. For example, “pharmaceutically-acceptable excipient” means, for example, an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. A person of ordinary skill in the art would be able to determine the appropriate timing, sequence and dosages of administration for particular Tregitope compositions of the present disclosure. The dosage of the compounds and compositions of the present disclosure will depend on the species, breed, age, size, treatment history, and health status of the animal (e.g., human) to be treated, as well as the route of administration, e.g., subcutaneous, intradermal, oral intramuscular or intravenous administration. The compounds and compositions of the instant disclosure can be administered as single doses or in repeated doses. The compounds and compositions of the instant disclosure can be administered alone, or can be administered simultaneously or sequentially administered with one or more further compositions, such as other porcine immunogenic or vaccine compositions. Where the compositions are administered at different times, the administrations may be separate from one another or overlapping in time.

Examples of pharmaceutically acceptable carriers, excipients or diluents include, but are not limited to demineralized or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methylcellulose, ethylcellulose, carboxymethylcellulose, carboxymethylcellulose sodium salt, or hydroxypropyl methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia; and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

In aspects, preferred examples of such carriers or diluents include, but are not limited to, water, saline, Ringer's solutions, dextrose solution, and 5% human serum albumin. Liposomes and non-aqueous vehicles such as fixed oils can also be used. The use of such media and compounds for pharmaceutically active substances is well known in the art. Except insofar as any conventional media or compound is incompatible with the Tregitope-blood component conjugates and/or the modified polypeptides used to form the Tregitope-blood component conjugates of the present disclosure and as previously described above, use thereof in the compositions is contemplated. Supplementary active compounds can also be incorporated into the compositions.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde. Further adjuvants may include, but are not limited to, poly-ICLC, 1018 ISS, aluminum salts, Amplivax, AS 15, BCG, CP-870,893, CpG7909, CyaA, dSLIM, GM-CSF, IC30, IC31, Imiquimod, ImuFact IMP321, IS Patch, ISS, ISCOMATRTX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In aspects of the pharmaceutical compositions or vaccines as disclosed herein, the adjuvant comprises poly-ICLC. The TLR9 agonist CpG and the synthetic double-stranded RNA (dsRNA) TLR3 ligand poly-ICLC are two of the most promising vaccine adjuvants currently in clinical development. In preclinical studies, poly-ICLC appears to be the most potent TLR adjuvant when compared to LPS and CpG. This appears due to its induction of pro-inflammatory cytokines and lack of stimulation of IL-10, as well as maintenance of high levels of co-stimulatory molecules in DCs. Poly-ICLC is a synthetically prepared double-stranded RNA consisting of polyI and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and mixed production of type I interferons, cytokines, and chemokines.

Examples of freeze-drying stabilizer may be for example carbohydrates such as sorbitol, mannitol, starch, sucrose, dextran or glucose, proteins such as albumin or casein, and derivatives thereof.

In aspects, the Tregitope-blood component conjugates and/or the modified polypeptides used to form the Tregitope-blood component conjugates of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein) are formulated to be compatible with their intended route of administration. The Tregitope-blood component conjugates and/or the modified polypeptides used to form the Tregitope-blood component conjugates of the present disclosure can be administered by parenteral, topical, intravenous, oral, subcutaneous, intra-arterial, intradermal, transdermal, rectal, intracranial, intrathecal, intraperitoneal, intranasal; vaginally; intramuscular route, or as inhalants. In aspects, the Tregitope-blood component conjugates and/or the modified polypeptides used to form the Tregitope-blood component conjugates of the present disclosure can be injected directly into a particular tissue where deposits have accumulated, e.g., intracranial injection. In other aspects, intramuscular injection or intravenous infusion may be used for administration of the Tregitope-blood component conjugates and/or the modified polypeptides used to form the Tregitope-blood component conjugates of the present disclosure. In some methods, the Tregitope-blood component conjugates and/or the modified polypeptides used to form the Tregitope-blood component conjugates of the present disclosure are injected directly into the cranium. In some methods, the Tregitope-blood component conjugates and/or the modified polypeptides used to form the Tregitope-blood component conjugates of the present disclosure are administered as a sustained release composition or device, such as but not limited to a Medipad™ device. In aspects, the compounds and compositions of the present disclosure are administered intradermally, e.g., by using a commercial needle-free high-pressure device such as Pulse NeedleFree technology (Pulse 50™ Micro Dose Injection System, Pulse NeedleFree Systems; Lenexa, Kans., USA). In aspects, said commercial needle-free high-pressure device (e.g., Pulse NeedleFree technology) confers one or more of the following benefits: non-invasive, reduces tissue trauma, reduces pain, requires a smaller opening in the dermal layer to deposit the composition in the subject (e.g., only requires a micro skin opening), instant dispersion of the composition, better absorption of the composition, greater dermal exposure to the composition, and/or reduced risk of sharps injury.

In aspects, the Tregitope-blood component conjugates and/or the modified polypeptides used to form the Tregitope-blood component conjugates of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein) can optionally be administered in combination with other agents that are at least partly effective in treating various medical conditions as described herein. For example, in the case of treatment and/or prevention of T1D in a subject, the Tregitope-blood component conjugates and/or the modified polypeptides used to form the Tregitope-blood component conjugates of the present disclosure can also be administered in conjunction with other agents provide temporary relief of T1D, such as insulin.

In aspects, solutions or suspensions used for parenteral, intradermal, or subcutaneous application can include, but are not limited to, the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. Examples of excipients can include starch, glucose, lactose, sucrose, gelatin, malt, rice, flour, chalk, silica gel, water, ethanol, DMSO, glycol, propylene, dried skim milk, and the like. The composition can also contain pH buffering reagents, and wetting or emulsifying agents.

In aspects, the parenteral preparation can be enclosed in ampoules, disposable syringes or multiple dose vials made of glass or plastic.

In aspects, pharmaceutical compositions or formulations suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition is sterile and should be fluid to the extent that easy syringeability exists. It is stable under the conditions of manufacture and storage and is preserved against the contaminating action of microorganisms such as bacteria and fungi. In aspects, Tregitopes formulations may include aggregates, fragments, breakdown products and post-translational modifications, to the extent these impurities bind HLA and present the same TCR face to cognate T cells they are expected to function in a similar fashion to pure Tregitopes. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as manitol, sorbitol, sodium chloride in the composition. Prolonged absorption of the injectable compositions can be brought about by including in the composition a compound that delays absorption, e.g., aluminum monostearate and gelatin.

In aspects, sterile injectable solutions can be prepared by incorporating the Tregitope-blood component conjugates and/or the modified polypeptides used to form the Tregitope-blood component conjugates of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein) in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Further, the Tregitope-blood component conjugates and/or the modified polypeptides used to form the Tregitope-blood component conjugates of the present disclosure can be administered in the form of a depot injection or implant preparation that can be formulated in such a manner as to permit a sustained or pulsatile release of the active ingredient.

In aspects, oral compositions generally include an inert diluent or an edible carrier and can be enclosed in gelatin capsules or compressed into tablets. In aspects, for the purpose of oral therapeutic administration, the binding agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. In aspects, the tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel or corn starch; a lubricant such as magnesium stearate or Sterotes; a glidant such as colloidal silicon dioxide; a sweetening compound such as sucrose or saccharin; or a flavoring compound such as peppermint, methyl salicylate or orange flavoring.

For administration by inhalation, the Tregitope-blood component conjugates and/or the modified polypeptides used to form the Tregitope-blood component conjugates of the present disclosure can be delivered in the form of an aerosol spray from pressured container or dispenser that contains a suitable propellant, e.g., a gas such as carbon dioxide, or a nebulizer.

In aspects, systemic administration of the Tregitope-blood component conjugates and/or the modified polypeptides used to form the Tregitope-blood component conjugates of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein) can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the Tregitope-blood component conjugates and/or the modified polypeptides used to form the Tregitope-blood component conjugates of the present disclosure may be formulated into ointments, salves, gels, or creams and applied either topically or through transdermal patch technology, as generally known in the art.

In aspects, the Tregitope-blood component conjugates and/or the modified polypeptides used to form the Tregitope-blood component conjugates of the present disclosure can also be prepared in the form of suppositories (e.g., with conventional suppository bases such as cocoa butter and other glycerides) or retention enemas for rectal delivery.

In aspects, the Tregitope-blood component conjugates and/or the modified polypeptides used to form the Tregitope-blood component conjugates of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein) are prepared with carriers that protect the Tregitope compositions against rapid elimination from the body, such as a controlled-release formulation, including implants and microencapsulated delivery systems. Biodegradable, biocompatible polymers can be used, such as, for example, ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art (U.S. Pat. No. 4,522,811, which is herein incorporated by reference in its entirety). In aspects, the Tregitope-blood component conjugates and/or the modified polypeptides used to form the Tregitope-blood component conjugates of the present disclosure can be implanted within or linked to a biopolymer solid support that allows for the slow release of the Tregitope compositions to the desired site.

In aspects, it is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of binding agent calculated to produce the desired therapeutic effect in association with the required pharmaceutical carrier. The specification for the dosage unit forms of the instant disclosure are dictated by and directly dependent on the unique characteristics of the binding agent and the particular therapeutic effect to be achieved, and the limitations inherent in the art of compounding such Tregitope-blood component conjugates and/or the modified polypeptides used to form the Tregitope-blood component conjugates of the present disclosure for the treatment of a subject.

### Methods of Use

Stimulating regulatory T cells with the Tregitope-blood component conjugates and/or the modified polypeptides used to form the Tregitope-blood component conjugates of the present disclosure (as well as nucleic acids as disclosed herein, including nucleic acids encoding such Tregitope-blood component conjugates and/or the modified polypeptides used to form the Tregitope-blood component conjugates, expression cassettes, plasmids, expression vectors, recombinant viruses, cells as disclosed herein, and/or pharmaceutical compositions or formulations as disclosed herein, referred to as “related compounds of the present disclosure” in this section) can stimulate, induce, and/or expand corresponding naturally occurring T_{Reg} populations (in aspects, including natural T_{Regs} and/or adaptive T_{Regs}) and in aspects results in increased secretion of one or more of the following cytokines and chemokines: IL-10, IL-35, TGF-β, TNF-α and MCP1. In aspects, stimulation can result in the increased expression of IL-2Rα by corresponding naturally occurring T_{Reg} populations (in aspects, including natural T_{Regs} and/or adaptive T_{Regs}) and deprivation of IL-2 to effector T cells. In further aspects, stimulation can result in increased perforin granzyme by corresponding naturally occurring T_{Reg} populations (in aspects, including natural T_{Regs} and/or adaptive T_{Regs}), which allows for such Treg populations to kill T effector cells and other immune stimulatory cells. In even further aspects, such stimulation can result in the generation of immune suppressive adenosine by corresponding naturally occurring T_{Reg} populations (in aspects, including natural T_{Regs} and/or adaptive T_{Regs}). In other aspects, such stimulation can result in corresponding naturally occurring T_{Reg} populations (in aspects, including natural T_{Regs} and/or adaptive T_{Regs}) binding to and removing costimulatory molecules on dendritic cells, resulting the inhibition of dendritic cell function. Further, in aspects, such stimulation can result in T_{Reg} induced upregulation of checkpoint molecules on dendritic cells and other cell populations, e.g. but not limited to endothelial cells, by corresponding naturally occurring T_{Reg} populations (in aspects, including natural T_{Regs} and/or adaptive T_{Regs}). In additional aspects, such stimulation can result in T_{reg} stimulation of B-regulatory cells. B-regulatory cells (“B-regs”) are cells that are responsible for the anti-inflammatory effect, which is characterized by the expression of CD1d, CD5, and the secretion of IL-10. B-regs are also identified by expression of Tim-1 and can be induced through Tim-1 ligation to promote tolerance. The ability of being B-regs was shown to be driven by many stimulatory factors such as toll-like receptors, CD40-ligand and others. However, full characterization of B-regs is ongoing. B-regs also express high levels of CD25, CD86, and TGF-β. The increased secretion of such regulatory cytokines and chemokines by regulatory T cells, as well as other activities described above, are hallmarks of regulatory T cells. In aspects, regulatory T cells activated by the Tregitope compositions of the present disclosure may express a CD4+CD25+FOXP3 phenotype. Regulatory T cells activated by the Tregitope-blood component conjugates and/or the modified polypeptides used to form the Tregitope-blood component conjugates of the present disclosure directly suppress T-effector immune responses ex vivo as measured by decreased antigen-specific Th1- or Th2-associated cytokine levels, principally INF-□, IL-4, and IL-5, and by decreased proliferation and/or effector function of antigen-specific T effector cells as measured by CFSE dilution and/or cytolytic activity. In aspects, regulatory T cells activated by the Tregitope-blood component conjugates and/or the modified polypeptides used to form the Tregitope-blood component conjugates of the present disclosure directly suppress T effector immune responses in vivo as measured by decreased antigen-specific Th1- or Th2-associated cytokine levels (as measured by Elisa assay), decreased antigen-specific T effector cell levels (as measured by EliSpot assay), decreased cytolytic activity, and/or decreased antibody titers for protein antigens.

In aspects, natural regulatory T cells activated by the Tregitope-blood component conjugates and/or the modified polypeptides used to form the Tregitope-blood component conjugates of the present disclosure stimulate the development of adaptive T_{Reg} cells. In aspects, co-incubating peripheral T cells with the Tregitope-blood component conjugates and/or the modified polypeptides used to form the Tregitope-blood component conjugates of the present disclosure in the presence of antigen results in the expansion of antigen-specific CD4+/CD25+ T cells, upregulates the expression of the Foxp3 gene or Foxp3 protein in those cells and suppresses the activation of antigen-specific T effector cells in vitro. In aspects, the Tregitope-blood component conjugates and/or the modified polypeptides used to form the Tregitope-blood component conjugates of the present disclosure may result in the activation and/or expansion of T regulatory type 1 (Tr1) cells. Tr1 cells have strong immunosuppressive capacity in several immune-mediated diseases (Roncarolo and Battaglia, 2007, Nat Rev Immunol 7, 585-598; Roncarolo et al., 2011, Immunol Rev 241, 145-163; Pot et al., 2011, Semin Immunol 23, 202-208). The secretion of high levels of IL-10, and the killing of myeloid antigen-presenting cells (APCs) via Granzyme B are the main mechanisms of Tr1-mediated suppression (Groux et al., 1997, Nature 389, 737-742; Magnani et al., 2011 Eur J Immunol 41, 1652-1662). Tr1 cells are distinguished from T helper (T_{H})1, T_{H}2, and T_{H}17 cells by their unique cytokine profile and the regulatory function. Tr1 cells have been shown secrete higher levels of IL-10 than IL-4 and IL-17, the hallmark cytokines of T_{H}2 and T_{H}17 cells, respectively. Tr1 cells can also secrete low levels of IL-2 and, depending on the local cytokine milieu, can produce variable levels of IFN-γ, together, the key T_{H}1 cytokines (Roncarolo et al., 2011, Immunol Rev 241, 145-163). FOXP3 is not a biomarker for Tr1 cells since its expression is low and transient upon activation. IL-10-producing Tr1 cells express ICOS (Haringer et al., 2009, J Exp Med 206, 1009-1017) and PD-1 (Akdis et al., 2004, J Exp Med 199, 1567-1575), but these markers are not specific (Maynard et al., 2007, Nat Immunol 8, 931-941). CD49b, the α2 integrin subunit of the very-late-activation antigen (VLA)-2, has been proposed as a marker for IL-10-producing T cells (Charbonnier et al., 2006, J Immunol 177, 3806-3813); but it is also expressed by human T_{H}17 cells (Boisvert et al., 2010, Eur J Immunol 40, 2710-2719). Moreover, murine CD49b⁺ T cells secrete IL-10 (Charbonnier et al., 2006, J Immunol 177, 3806-3813) but also pro-inflammatory cytokines (Kassiotis et al., 2006, J Immunol 177, 968-975). Lymphocyte activation gene-3 (LAG-3), a CD4 homolog that binds with high affinity to MHC class II molecules, is expressed by murine IL-10-producing CD4⁺ T cells (Okamura et al., 2009, Proc Natl Acad Sci USA 106, 13974-13979), but also by activated effector T cells (Workman and Vignali, 2005, J Immunol 174, 688-695; Bettini et al., 2011, J Immunol 187, 3493-3498; Bruniquel et al., 1998, Immunogenetics 48, 116-124; Lee et al., 2012, Nat Immunol 13, 991-999) and by FOXP3⁺ regulatory T cells (Tregs) (Camisaschi et al., 2010, J Immunol 184, 6545-6551). It was recently shown that human Tr1 cells express CD226 (DNAM-1), which is involved in the specific killing of myeloid APCs (Magnani et al., 2011 Eur J Immunol 41, 1652-1662). In further aspects, the Tregitope-blood component conjugates and/or the modified polypeptides used to form the Tregitope-blood component conjugates of the present disclosure (and related compounds of the present disclosure) may result in the activation and/or expansion of TGF-β secreting Th3 cells, regulatory NKT cells, regulatory CD8⁺ T cells, double negative regulatory T cells. “Th3 cells” refer to cells having the following phenotype CD4⁺FoxP3⁺ and capable of secreting high levels TGF-β upon activation, amounts of IL-4 and IL-10 and no IFN-γ or IL-2. These cells are TGF-β derived. “Regulatory NKT cells” refers to cells having the following phenotype at rest CD161⁺CD56⁺CD16⁺ and a Vα24/Vβ11 TCR. “Regulatory CD8⁺ T cells” refers to cells having the following phenotype at rest CD8⁺CD122⁺ and capable of secreting highs levels of IL-10 upon activation. “Double negative regulatory T cells” refers to cells having the following phenotype at rest TCRαβ⁺CD4⁻CD8⁻.

In aspects, the Tregitope-blood component conjugates and/or the modified polypeptides used to form the Tregitope-blood component conjugates of the present disclosure (and related compounds of the present disclosure) are useful for regulating immune response to monoclonal antibodies, protein therapeutics, self-antigens promoting autoimmune response, allergens, transplanted tissues, and in other applications where tolerance is the desired outcome.

In aspects, the Tregitopes of the Tregitope-blood component conjugates and/or the modified polypeptides used to form the Tregitope-blood component conjugates of the present disclosure can bind MHC class II molecules, engage TCR in context of MHC class II molecules and activate naturally occurring T_{Regs} (in aspects, including natural T_{Regs} and/or adaptive T_{Regs}).

Suppressing an Immune Response in a Subject in Need Thereof. In aspects, the present disclosure is directed to a method of stimulating, inducing, and/or expanding regulatory T-cells (in aspects, naturally occurring T_{Regs}, including natural T_{Regs} and/or adaptive T_{Regs}) in a subject in need thereof and/or suppressing an immune response in a subject in need thereof by administering to the subject a therapeutically effect amount of a Tregitope-blood component conjugate or a modified polypeptide used to form the Tregitope-blood component conjugates of the present disclosure (and related compounds of the present disclosure).

In aspects, the present disclosure is directed to a method of stimulating and/or inducing regulatory T-cells (e.g., naturally occurring T_{Regs} (in aspects, including natural T_{Regs} and/or adaptive T_{Regs})) to suppress an immune response in a subject in need thereof by administering to the subject a therapeutically effective amount of a Tregitope-blood component conjugate or modified polypeptide used to form the Tregitope-blood component conjugates of the present disclosure (and related compounds of the present disclosure). In aspects, the immune response is the result of one or more therapeutic treatments with at least one therapeutic protein, treatment with a vaccine (particularly in situations in which an adverse event results from the vaccination), or treatment with at least one antigen. In another aspect, the administration of a Tregitope composition of the present disclosure shifts one or more antigen presenting cells to a regulatory phenotype, one or more dendritic cells to a regulatory phenotype, decreases CD11c and HLA-DR expression in the dendritic cells and/or other antigen presenting cells

In aspects, the present disclosure is directed to a method for repressing and/or suppressing an immune response in a subject, comprising administering a therapeutically effective amount of a Tregitope-blood component conjugate or modified polypeptide used to form the Tregitope-blood component conjugates of the present disclosure (and related compounds of the present disclosure), wherein the Tregitope-blood component conjugate or modified polypeptide represses/suppresses the immune response. In aspects, the Tregitope-blood component conjugate or modified polypeptide represses and/or suppresses an innate immune response. In aspects, the Tregitope-blood component conjugate or modified polypeptide represses and/or suppresses an adaptive immune response. In aspects, the Tregitope-blood component conjugate or modified polypeptide represses and/or suppresses an effector T cell response. In aspects, the Tregitope-blood component conjugate or modified polypeptide represses and/or suppresses a memory T cell response. In aspects, the Tregitope-blood component conjugate or modified polypeptide represses and/or suppresses helper T cell response. In aspects, the Tregitope-blood component conjugate or modified polypeptide represses and/or suppresses B cell response. In aspects, the Tregitope-blood component conjugate or modified polypeptide represses and/or suppresses an nkT cell response.

In aspects, the present invention is directed to a method of suppressing an immune response, specifically an antigen specific immune response in a subject, through the administration of a therapeutically effective amount of a Tregitope-blood component conjugate or modified polypeptide (and related compounds of the present disclosure), wherein said Tregitope-blood component conjugate or modified polypeptide activates naturally occurring T_{Regs} (in aspects, including natural T_{Regs} and/or adaptive T_{Regs}, and in aspects CD4⁺/CD25⁺/FoxP3⁺ regulatory T-cells) or suppresses the activation of CD4⁺ T-cells, the proliferation of CD4⁺ and/or CD8⁺ T-cells, and/or suppresses the activation or proliferation of β-cells or nkT Cells. In aspects, a Tregitope-blood component conjugate or modified polypeptide of the present disclosure (and related compounds of the present disclosure) may be either covalently bound, non-covalently bound, or in admixture with a specific target antigen. In aspects, an administered Tregitope-blood component conjugate or modified polypeptide of the present disclosure that is covalently bound, non-covalently bound, or in admixture with a specific target antigen results in the diminution of immune response against the target antigen.

In aspects, the target antigen may be an autologous protein or protein fragment. In aspects, the target antigen may allogenic protein or protein fragments. In aspects, the target antigen may be a biologic medicine or fragments thereof. In aspects, the target antigen is a preproinsulin or fragments thereof. In aspects, the target antigen comprises, consists of, or consists essentially of one or more of SEQ ID NOS: 56-63. In aspects, the suppressive effect is mediated by natural T_{Regs}. In aspects, the suppressive effect is mediated by adaptive T_{Regs}.

### Methods of Preventing or Treating a Medical Condition

The present invention is directed to, for example methods of preventing or treating one or more medical conditions in a subject comprising administering a Tregitope-blood component conjugate or the modified polypeptide used to form the Tregitope-blood component conjugates as disclosed herein (and related compounds of the present disclosure), and preventing or treating the medical condition in a subject by said step of administering. In a preferred embodiment, the medical condition is Type 1 Diabetes.

The medical condition can be, for example, primary immunodeficiencies, immune-mediated thrombocytopenia, Kawasaki disease, hematopoietic stem cell transplantation in patients older than 20 years, chronic B-cell lymphocytic leukemia, and pediatric HIV type 1 infections. Specific examples include: (Hematology) aplastic anemia, pure red cell aplasia, Diamond-Blackfan anemia, autoimmune hemolytic anemia, hemolytic disease of the newborn, acquired factor VIII inhibitors, acquired von Willebrand disease, immune-mediated neutropenia, refractoriness to platelet transfusion, neonatal alloimmune/autoimmune thrombocytopenia, posttransfusion purpura, thrombotic thrombocytopenia purpura/hemolytic uremic syndrome; Infectious diseases, solid organ transplantation, surgery, trauma, burns, and HIV infection; (Neurology) epilepsy and pediatric intractable Guillain-Barré syndrome, chronic inflammatory demyelinating polyneuropathy, myasthenia gravis, Lambert-Eaton myasthenic syndrome, multifocal motor neuropathy, multiple sclerosis; (Obstetrics) recurrent pregnancy loss; (Pulmonology) asthma, chronic chest symptoms, rheumatology, rheumatoid arthritis (adult and juvenile), systemic lupus erythematosus, systemic vasculitides, dermatomyositis, polymyositis, inclusion-body myositis, wegener granulomatosis; (Miscellaneous) adrenoleukodystrophy, amyotrophic lateral sclerosis, Behçet syndrome, acute cardiomyopathy, chronic fatigue syndrome, congenital heart block, cystic fibrosis, autoimmune blistering dermatosis, diabetes mellitus, acute idiopathic dysautonomia, acute disseminated encephalomyelitis, endotoxemia, hemolytic transfusion reaction, hemophagocytic syndrome, acute lymphoblastic leukemia, lower motor neuron syndrome, multiple myeloma, human T cell lymphotrophic virus-1-associated myelopathy, nephritic syndrome, membranous nephropathy, nephrotic syndrome, euthyroid ophthalmopathy, opsoclonus-myoclonus, recurrent otitis media, paraneoplastic cerebellar degeneration, paraproteinemic neuropathy, parvovirus infection (general), polyneuropathy, organomegaly, endocrinopathy, M-protein, and skin changes (POEMS) syndrome, progressive lumbosacral plexopathy, lyme radiculoneuritis, Rasmussen syndrome, Reiter syndrome, acute renal failure, thrombocytopenia (nonimmune), streptococcal toxic shock syndrome, uveitis, and Vogt-Koyanagi-Harada syndrome.

In a particular aspect, the present invention is directed to, for example, methods of treating allergy, autoimmune disease, transplant-related disorders such as graft versus host disease, enzyme or protein deficiency disorders, hemostatic disorders (e.g., Hemophilia A, B, or C), cancers (particularly tumor associated autoimmunity), infertility, or infections (viral, bacterial, or parasitic). The Tregitope compounds or compositions of the present disclosure can be used with in conjunction with other proteins or compounds used for treating a subject with a medical condition in order to reduce adverse events or enhance the efficacy of the co-administered compound.

In a particular embodiment, the present disclosure is directed to, for example, methods of treating autoimmune disease, such as T1D. The Tregitope-blood component conjugates and/or the modified polypeptides used to form the Tregitope-blood component conjugates as disclosed herein (and related compounds of the present disclosure) can be used with in conjunction with other proteins or compounds used for treating a subject with a medical condition in order to reduce adverse events or enhance the efficacy of the co-administered compound.

Application to Allergy. Allergen-specific regulatory T cells play an important role in controlling the development of allergy and asthma. Naturally occurring TRegs (in aspects, including natural TRegs and/or adaptive TRegs, and in aspects CD4+/CD25+/FoxP3+ regulatory T-cells) have been shown to inhibit the inappropriate immune responses involved in allergic diseases. A number of recent studies indicate that regulatory T cells play an important role in controlling the overdevelopment of T-helper type 2 biased immune responses in susceptible individuals, not only in animal models, but in humans as well. Recent studies indicate that Tregs also suppress T cell co-stimulation by the secretion of TGF β and IL 10, suggesting an important role of Tregs in the regulation of allergic disorders. Impaired expansion of natural or adaptive regulatory T cells leads to the development of allergy, and treatment to induce allergen-specific Tregs would provide curative therapies for allergy and asthma. One strategy for both the prevention and therapy of asthma is the induction of Tregs. Animals can be protected from developing asthma by immune stimulation leading to Th1 or Treg responses. Accordingly, Tregitope compounds or compositions of the present disclosure are useful in methods for the prevention or treatment of allergy and/or asthma. As such, in aspects, the present disclosure is directed to a method of preventing or treating allergy and/or asthma in a subject, the method comprising administering a therapeutically-effective amount of a Tregitope-blood component conjugates and/or the modified polypeptides used to form the Tregitope-blood component conjugates as disclosed herein (and related compounds of the present disclosure), and preventing or treating allergy and/or asthma in a subject by said step of administering.

Application to Transplantation. The Tregitope-blood component conjugates and/or the modified polypeptides used to form the Tregitope-blood component conjugates as disclosed herein (and related compounds of the present disclosure) are useful to induce tolerance during the transplantation process, by promoting the development of cells that specifically down regulate immune responses against donor cells. Induction of Ag-specific TReg cells for treating organ-specific autoimmunity is an important therapeutic development, avoiding generalized immune suppression. In murine models of bone marrow transplantation, TRegs promote donor bone marrow engraftment and decrease the incidence and severity of graft versus host disease without abrogating the beneficial graft versus tumor immunologic effect. These findings, in concert with observations that TRegs in mice and humans share phenotypic and functional characteristics, have led to active investigations into the use of these cells to decrease complications associated with human hematopoietic cell transplantation. An imbalance of TRegs and effector T cells contributes to the development of graft versus host disease, however, the mechanisms of immunoregulation, in particular, the allorecognition properties of TRegs, their effects on and interaction with other immune cells, and their sites of suppressive activity, are not well understood.

Accumulating evidence from both humans and experimental animal models has implicated the involvement of TRegs in the development of graft versus host disease (GVHD). The demonstration that TRegs can separate GVHD from graft versus tumor (GVT) activity suggests that their immunosuppressive potential could be manipulated to reduce GVHD without detrimental consequence on GVT effect. Although a variety of T lymphocytes with suppressive capabilities have been reported, the two best-characterized subsets are the naturally arising, intrathymic-generated TRegs (natural TRegs) and the peripherally generated, inducible TRegs (inducible TRegs). Accordingly, Tregitope-blood component conjugates and/or the modified polypeptides used to form the Tregitope-blood component conjugates as disclosed herein (and related compounds of the present disclosure) are useful in methods for inducing tolerance during the transplantation process. As such, in aspects, the present disclosure is directed to a method of inducing tolerance during the transplantation process in a subject, the method comprising administering a therapeutically-effective amount of a Tregitope-blood component conjugates and/or the modified polypeptides used to form the Tregitope-blood component conjugates as disclosed herein (and related compounds of the present disclosure), and inducing tolerance during the transplantation process in a subject by said step of administering.

Application as a Tolerizing Agent and to Autoimmunity. In aspects, Tregitope-blood component conjugates and/or the modified polypeptides used to form the Tregitope-blood component conjugates as disclosed herein (and related compounds of the present disclosure) can be used as a tolerizing agents for immunogenic compounds (protein therapeutics) (Weber C A et al., (2009), Adv Drug Deliv, 61(11):965-76). This discovery has implications for the design of protein therapeutics. Thus, administration of an immunogenic compound (e.g., protein therapeutic, such as but not limited to antibody (e.g., monoclonal antibody), autologous cytokine, or foreign protein) in conjunction with a Tregitope-blood component conjugates and/or the modified polypeptides used to form the Tregitope-blood component conjugates as disclosed herein (and related compounds of the present disclosure) suppresses adverse T effector immune responses. In vivo, TRegs act through dendritic cells to limit autoreactive T cell activation, thus preventing their differentiation and acquisition of effector functions. By limiting the supply of activated pathogenic cells, TRegs prevent or slow down the progression of autoimmune diseases. This protective mechanism appears, however, insufficient in autoimmune individuals, likely because of a shortage of TRegs cells and/or the development and accumulation of TReg-resistant pathogenic T cells over the long disease course. Thus, restoration of self-tolerance in these patients may require purging of pathogenic T cells along with infusion of TRegs with increased ability to control ongoing tissue injury. Organ-specific autoimmune conditions, such as thyroiditis and insulin-dependent diabetes mellitus have been attributed to a breakdown of this tolerance mechanism (Mudd P A et al., (2006), Scand J Immunol, 64(3):211-8). Accordingly, Tregitope-blood component conjugates and/or the modified polypeptides used to form the Tregitope-blood component conjugates as disclosed herein (and related compounds of the present disclosure) are useful in methods for the prevention or treatment of autoimmunity. As such, in aspects, the present disclosure is directed to a method of preventing or treating autoimmunity in a subject, the method comprising administering a therapeutically-effective amount of a Tregitope-blood component conjugates and/or the modified polypeptides used to form the Tregitope-blood component conjugates as disclosed herein (and related compounds of the present disclosure), and preventing and/or treating autoimmunity in a subject by said step of administering.

Application to Diabetes. Type 1 (juvenile) diabetes is an organ-specific autoimmune disease resulting from destruction of insulin-producing pancreatic beta-cells. In non-diabetics, islet cell antigen-specific T cells are either deleted in thymic development or are converted to T regulatory cells that actively suppress effector responses to islet cell antigens. In juvenile diabetics and in the NOD mouse model of juvenile diabetes, these tolerance mechanisms are missing. In their absence, islet cell antigens are presented by human leukocyte antigen (HLA) class I and II molecules and are recognized by CD8(+) and CD4(+) auto-reactive T cells. Destruction of islet cells by these auto-reactive cells eventually leads to glucose intolerance. Co-administration of Tregitopes and islet cell antigens leads to the activation of naturally occurring T regulatory cells and the conversion of existing antigen specific effector T cell to a regulatory phenotype. In this way, deleterious autoimmune response is redirected leading to the induction of antigen-specific adaptive tolerance. Modulation of autoimmune responses to autologous epitopes by induction of antigen-specific tolerance can prevent ongoing beta-cell destruction. Accordingly, Tregitope-blood component conjugates and/or the modified polypeptides used to form the Tregitope-blood component conjugates as disclosed herein (and related compounds of the present disclosure) are useful in methods for the prevention or treatment of diabetes. As such, in aspects, the present disclosure is directed to a method of preventing or treating diabetes in a subject, the method comprising administering a therapeutically-effective amount of a Tregitope-blood component conjugate or the modified polypeptide used to form the Tregitope-blood component conjugates as disclosed herein (and related compounds of the present disclosure), and preventing or treating diabetes in a subject by said step of administering.

Application to Hepatitis B (HBV) infection. Chronic HBV is usually either acquired (by maternal fetal transmission) or can be a rare outcome of acute HBV infection in adults. Acute exacerbations of chronic hepatitis B (CH-B) are accompanied by increased cytotoxic T cell responses to hepatitis B core and e antigens (HBcAg/HBeAg). In a recent study, the SYFPEITHI T cell epitope mapping system was used to predict MHC class II-restricted epitope peptides from the HBcAg and HbeAg (Feng I C et al., (2007), J Biomed Sci, 14(1):43-57). MHC class II tetramers using the high scoring peptides were constructed and used to measure TReg and CTL frequencies. The results showed that TReg cells specific for HBcAg declined during exacerbations accompanied by an increase in HBcAg peptide-specific cytotoxic T cells. During the tolerance phase, FOXp3-expressing TReg cell clones were identified. These data suggest that the decline of HbcAg TReg T cells accounts for the spontaneous exacerbations on the natural history of chronic hepatitis B virus infection. Accordingly, Tregitope-blood component conjugate or the modified polypeptide used to form the Tregitope-blood component conjugates as disclosed herein (and related compounds of the present disclosure) are useful in methods for the prevention or treatment of chronic hepatitis B viral infection. As such, in aspects, the present disclosure is directed to a method of preventing or treating a viral infection (e.g., HBV infection) in a subject, the method comprising administering a therapeutically-effective amount of a Tregitope-blood component conjugate or the modified polypeptide used to form the Tregitope-blood component conjugates as disclosed herein (and related compounds of the present disclosure), and preventing and/or treating said viral infection in a subject by said step of administering.

Application to SLE. A TReg epitope that plays a role in Systemic Lupus Erythematosus (SLE) or Sjögren's syndrome has been defined. Binding assays with soluble HLA class II molecules and molecular modeling experiments indicated that the epitope behaves as promiscuous epitope and binds to a large panel of human DR molecules. In contrast to normal T cells and T cells from non-lupus autoimmune patients, PBMCs from 40% of randomly selected lupus patients contain T cells that proliferate in response to peptide 131-151. Alteration of the ligand modified the T cell response, suggesting that several populations of T cells responding to this peptide exist, among which may be TReg cells. T regulatory epitopes have also been defined in Sjögren's syndrome. Accordingly, Tregitope-blood component conjugate or the modified polypeptide used to form the Tregitope-blood component conjugates as disclosed herein (and related compounds of the present disclosure) administered in combination with particular SLE epitopes are useful in methods for the prevention or treatment of SLE. As such, in aspects, the present disclosure is directed to a method of preventing or treating SLE in a subject, the method comprising administering a therapeutically-effective amount of a Tregitope-blood component conjugate or the modified polypeptide used to form the Tregitope-blood component conjugates as disclosed herein (and related compounds of the present disclosure) in combination with SLE epitopes, and preventing and/or treating SLE in a subject by said step of administering.

Application to Autoimmune Thyroiditis. Autoimmune Thyroiditis is a condition that occurs when antibodies arise to self-thyroid peroxidase and/or thyroglobulin, which cause the gradual destruction of follicles in the thyroid gland. HLA DR5 is closely associated with the disease. Accordingly, Tregitope-blood component conjugate or the modified polypeptide used to form the Tregitope-blood component conjugates as disclosed herein (and related compounds of the present disclosure) administered in combination with thyroid peroxidase and/or thyroglobulin TSHR or portions thereof are useful in methods for the prevention or treatment of autoimmune thyroiditis. As such, in aspects, the present disclosure is directed to a method of preventing or treating autoimmune thyroiditis in a subject, the method comprising administering a therapeutically-effective amount of a Tregitope-blood component conjugate or the modified polypeptide used to form the Tregitope-blood component conjugates as disclosed herein (and related compounds of the present disclosure) in combination with thyroid peroxidase and/or thyroglobulin TSHR or portions thereof, and preventing and/or treating autoimmune thyroiditis in a subject by said step of administering. In further aspects, Tregitope compositions of the present disclosure administered in combination with TSHR or other Graves' disease antigens or portions thereof are useful in methods for the prevention or treatment of Grave's disease. Graves' disease is an autoimmune disorder that is characterized by antibodies to self-thyroid stimulating hormone receptor (TSHR) leading to leading to hyperthyroidism, or an abnormally strong release of hormones from the thyroid gland. Several genetic factors can influence susceptibility to Graves' disease. Females are much more likely to contract the disease than males; White and Asian populations are at higher risk than black populations and HLA DRB1 0301 is closely associated with the disease. As such, in aspects, the present disclosure is directed to a method of preventing or treating Grave's disease in a subject, the method comprising administering a therapeutically-effective amount of a Tregitope-blood component conjugate or the modified polypeptide used to form the Tregitope-blood component conjugates as disclosed herein (and related compounds of the present disclosure) in combination with TSHR or other Graves' disease antigens or portions thereof, and preventing and/or treating Grave's disease in a subject by said step of administering.

Kits. The methods described herein can be performed, e.g., by utilizing pre-packaged kits comprising a Tregitope-blood component conjugate or the modified polypeptide used to form the Tregitope-blood component conjugates as disclosed herein (and related compounds of the present disclosure), which can be conveniently used, e.g., in clinical settings to treat subjects exhibiting symptoms or family history of a medical condition described herein. In one embodiment, the kit further comprises instructions for use of Tregitope-blood component conjugate or the modified polypeptide used to form the Tregitope-blood component conjugates as disclosed herein (and related compounds of the present disclosure) to treat subjects exhibiting symptoms or family history of a medical condition described herein.

Aspects

A is 1^{st} aspect is directed to a Tregitope-blood component conjugate comprising: a blood component linked to a modified polypeptide, said modified polypeptide having a reactive moiety attached thereto and said modified polypeptide comprising one or more regulatory T cell epitopes.

A 2^{nd} aspect is directed to a Tregitope-blood component conjugate of aspect 1, wherein said one or more regulatory T cell epitopes consists of one or more amino acid sequences selected from the group consisting of SEQ ID NOS: 1-55, and/or fragments and variants thereof, and optionally 1 to 12 additional amino acids distributed in any ratio on the N terminus and/or C-terminus of the polypeptide of SEQ ID NOS. 1-55.

A 3^{rd} aspect is directed to a Tregitope-blood component conjugate of aspect 1, wherein said one or more regulatory T cell epitopes consists essentially of one or more amino acid sequences selected from the group consisting of SEQ ID NOS: 1-55, and/or fragments and variants thereof, and optionally 1 to 12 additional amino acids distributed in any ratio on the N terminus and/or C-terminus of the polypeptide of SEQ ID NOS. 1-55.

A 4^{th} aspect is directed to a Tregitope-blood component conjugate of aspect 1, wherein said one or more regulatory T cell epitopes comprises of one or more amino acid sequences selected from the group consisting of SEQ ID NOS: 1-55, and/or fragments and variants thereof, and optionally 1 to 12 additional amino acids distributed in any ratio on the N terminus and/or C-terminus of the polypeptide of SEQ ID NOS. 1-55.

A 5^{th} aspect is directed to a Tregitope-blood component conjugate of aspect 1, wherein said one or more regulatory T cell epitopes comprises SEQ ID NO: 1.

A 6^{th} aspect is directed to a Tregitope-blood component conjugate of aspect 1, wherein said one or more regulatory T cell epitopes comprises SEQ ID NO: 28.

A 7^{th} aspect is directed to a Tregitope-blood component conjugate of any one of aspects 1-6, wherein said blood component is albumin.

An 8^{th} aspect is directed to a Tregitope-blood component conjugate of any one of aspects 1-6, wherein said blood component is human serum albumin.

A 9^{th} aspect is directed to a Tregitope-blood component conjugate of any one of aspects 1-8, wherein said modified polypeptide further comprises a T1Dgen peptide.

A 1^{th} aspect is directed to a Tregitope-blood component conjugate of claim 9, wherein the T1Dgen peptide comprise one or more amino sequences selected from the group consisting of SEQ ID NOS: 56-63.

An 11^{th} aspect is directed to a Tregitope-blood component conjugate of any one of aspects 1-10, wherein the reactive moiety is attached to the amino terminal amino acid of the modified polypeptide.

A 12^{th} aspect is directed to a Tregitope-blood component conjugate of any one of aspects 1-10, wherein the reactive moiety is attached to the carboxy terminal amino acid of the modified polypeptide.

A 13^{th} aspect is directed to a Tregitope-blood component conjugate of any one of aspects 1-10, wherein the reactive moiety is attached to an amino acid positioned between the amino terminal amino acid and the carboxy terminal amino acid of the modified polypeptide.

A 14^{th} aspect is directed to a Tregitope-blood component conjugate of any one of aspects 1-13, wherein the reactive moiety is a succinimidyl or maleimido group.

A 15^{th} aspect is directed to a Tregitope-blood component conjugate of any one of aspects 1-14, wherein the reactive moiety is a 3-maleimidoproprionic acid moiety.

A 16^{th} aspect is directed to a Tregitope-blood component conjugate of any one of aspects 1-15, wherein the conjugation between the blood component and the modified polypeptide is a maleimide linkage.

A 17^{th} aspect is directed to a modified polypeptide, said modified polypeptide having a reactive moiety attached thereto and said modified polypeptide comprising one or more regulatory T cell epitopes.

An 18^{th} aspect is directed to a modified polypeptide of aspect 17, wherein said one or more regulatory T cell epitopes consists of one or more amino acid sequences selected from the group consisting of SEQ ID NOS: 1-55, and/or fragments and variants thereof, and optionally 1 to 12 additional amino acids distributed in any ratio on the N terminus and/or C-terminus of the polypeptide of SEQ ID NOS. 1-55.

A 19^{th} aspect is directed to the modified polypeptide of aspect 17, wherein said one or more regulatory T cell epitopes consists essentially of one or more amino acid sequences selected from the group consisting of SEQ ID NOS: 1-55, and/or fragments and variants thereof, and optionally 1 to 12 additional amino acids distributed in any ratio on the N terminus and/or C-terminus of the polypeptide of SEQ ID NOS. 1-55.

A 20^{th} aspect is directed to a modified polypeptide of aspect 17, wherein said one or more regulatory T cell epitopes comprises of one or more amino acid sequences selected from the group consisting of SEQ ID NOS: 1-55, and/or fragments and variants thereof, and optionally 1 to 12 additional amino acids distributed in any ratio on the N terminus and/or C-terminus of the polypeptide of SEQ ID NOS. 1-55.

A 21^{st} aspect is directed to a modified polypeptide of aspect 17, wherein said one or more regulatory T cell epitopes comprises SEQ ID NO: 1.

A 22^{nd} aspect is directed to a modified polypeptide of aspect 17, wherein said one or more regulatory T cell epitopes comprises SEQ ID NO: 28.

A 23^{rd} aspect is directed to a modified polypeptide of any one of aspects 17-22, wherein said modified polypeptide further comprises a T1Dgen peptide.

A 24^{th} aspect is directed to a modified polypeptide of aspect 23, wherein the T1Dgen peptide comprise one or more amino sequences selected from the group consisting of SEQ ID NOS: 56-63.

A 25^{th} aspect is directed to a modified polypeptide of any one of aspects 17-24, wherein the reactive moiety is attached to the amino terminal amino acid of the modified polypeptide.

A 26^{th} aspect is directed to a modified polypeptide of any one of aspects 17-24, wherein the reactive moiety is attached to the carboxy terminal amino acid of the modified polypeptide.

A 27^{th} aspect is directed to a modified polypeptide of any one of aspects 17-24, wherein the reactive moiety is attached to an amino acid positioned between the amino terminal amino acid and the carboxy terminal amino acid of the modified polypeptide.

A 28^{th} aspect is directed to a modified polypeptide of any one of aspects 17-27, wherein the reactive moiety is a succinimidyl or maleimido group.

A 29^{th} aspect is directed to a modified polypeptide of any one of aspects 17-28, wherein the reactive moiety is a 3-maleimidoproprionic acid moiety.

A 30^{th} aspect is directed to a method for suppressing an autoimmune response characteristic of T1D in a subject in need thereof, the method comprising administering to the subject a Tregitope-blood component conjugate of any one of aspects 1-16.

A 31^{st} aspect is directed to a method for suppressing an autoimmune response characteristic of T1D in a subject in need thereof, the method comprising administering to the subject a modified polypeptide of any one of aspects 17-29.

A 32^{nd} aspect is directed to a method according to any one of aspects 30-31, wherein the administration shifts one or more antigen presenting cells to a regulatory phenotype.

A 33^{rd} aspect is directed to a method according to any one of aspects 30-31, wherein the administration shifts one or more dendritic cells to a regulatory phenotype.

A 34^{th} aspect is directed to a method according to any one of aspects 30-31, wherein the regulatory phenotype is characterized by a decrease in CD1c and HLA-DR expression in the dendritic cells or other antigen presenting cells.

A 35^{th} aspect is directed to a method according to any one of aspects 30-31, wherein the administration of the regulatory T-cell epitope shifts one or more T cells to a regulatory phenotype.

A 36^{th} aspect is directed to a method according to any of aspects 30-31, wherein the administration of the one or more regulatory T-cell epitopes activates CD4+/CD25+/FoxP3+ regulatory T-cells.

A 37^{th} aspect is directed to a method according to any of aspects 30-31, wherein the administration suppresses an immune response selected from the group consisting of an innate immune response, an adaptive immune response, an effector T cell response, a memory T cell response, a helper T cell response, a B cell response, a ηκT cell response, or any combination thereof.

A 38^{th} aspect is directed to a pharmaceutical composition comprising a Tregitope-blood component conjugate according to any one of aspects 1-16 and a carrier, excipient, and/or adjuvant.

A 39^{th} aspect is directed to a pharmaceutical composition comprising a modified polypeptide capable of forming a Tregitope-blood component conjugate according to any one of claims 17-29 and a carrier, excipient, and/or adjuvant.

### EXEMPLIFICATION

The examples that follow are not to be construed as limiting the scope of the invention in any manner. In light of the present disclosure, numerous embodiments within the scope of the claims will be apparent to those of ordinary skill in the art.

(1) In-Silico Identification of a Tregitope Composition

T cells specifically recognize epitopes presented by antigen presenting cells (APCs) in the context of MHC (Major Histocompatibility Complex) Class II molecules. These T-helper epitopes can be represented as linear sequences comprising 7 to 30 contiguous amino acids that fit into the MHC Class II binding groove. A number of computer algorithms have been developed and used for detecting Class II epitopes within protein molecules of various origins (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A S et al., (2003), Vaccine, 21(27-30):4486-504). These “in silico” predictions of T-helper epitopes have been successfully applied to the design of vaccines and the de-immunization of therapeutic proteins, i.e. antibody-based drugs, Fc fusion proteins, anticoagulants, blood factors, bone morphogenetic proteins, engineered protein scaffolds, enzymes, growth factors, hormones, interferons, interleukins, and thrombolytics (Dimitrov D S, (2012), Methods Mol Biol, 899:1-26).

The EpiMatrix™ system (EpiVax, Providence, R.I.) is a set of predictive algorithms encoded into computer programs useful for predicting class I and class II HLA ligands and T cell epitopes. The EpiMatrix™ system uses 20×9 coefficient matrices in order to model the interaction between specific amino acids (20) and binding positions within the HLA molecule (9). In order to identify putative T cell epitopes resident within any given input protein, the EpiMatrix™ System first parses the input protein into a set of overlapping 9-mer frames where each frame overlaps the last by eight amino acids. Each frame is then scored for predicted affinity to one or more common alleles of the human HLA molecule; typically DRB1*0101, DRB1*0301, DRB1*0401, DRB1*0701, DRB1*0801, DRB1*1101, DRB1*1301, and DRB1*1501 (Mack et al., (2013), Tiss Antig, 81(4):194-203). Briefly, for any given 9-mer peptide specific amino acid codes (one for each of 20 naturally occurring amino acids) and relative binding positions (1-9) are used to select coefficients from the predictive matrix. Individual coefficients are derived using a proprietary method similar to, but not identical to, the pocket profile method first developed by Sturniolo (Sturniolo T et al., 1999, Nat Biotechnol, 17(6):555-61, herein incorporated by reference in its entirety). Individual coefficients are then summed to produce a raw score. EpiMatrix™ raw scores are then normalized with respect to a score distribution derived from a very large set of randomly generated peptide sequences. The resulting “Z” scores are normally distributed and directly comparable across alleles.

EpiMatrix™ peptide scoring. It was determined that any peptide scoring above 1.64 on the EpiMatrix™ “Z” scale (approximately the top 5% of any given peptide set) has a significant chance of binding to the MHC molecule for which it was predicted. Peptides scoring above 2.32 on the scale (the top 1%) are extremely likely to bind; most published T cell epitopes fall within this range of scores. Previous studies have also demonstrated that EpiMatrix™ accurately predicts published MHC ligands and T cell epitopes (De Groot A S, Martin W. Reducing risk, improving outcomes: bioengineering less immunogenic protein therapeutics. Clin Immunol. 2009 May; 131(2):189-201.doi: 10.1016/j.clim.2009.01.009. Epub 2009 Mar. 6, herein incorporated by reference in its entirety).

Identification of promiscuous T cell Epitope Clusters. Potential T cell epitopes are not randomly distributed throughout protein sequences but instead tend to “cluster.” T cell epitope “clusters” range from 9 to roughly 30 amino acids in length and, considering their affinity to multiple alleles and across multiple frames, contain anywhere from 4 to 40 binding motifs. Following epitope mapping, the result set produced by the EpiMatrix™ algorithm is screened for the presence of T cell epitope clusters and EpiBars™ by using a proprietary algorithm known as Clustimer™. Briefly, the EpiMatrix™ scores of each 9-mer peptide analyzed are aggregated and checked against a statistically derived threshold value. High scoring 9mers are then extended one amino acid at a time. The scores of the extended sequences are then re-aggregated and compared to a revised threshold value. The process is repeated until the proposed extension no longer improves the overall score of the cluster. In aspects, Tregitope(s) may be identified by the Clustimer-™ algorithm as T cell epitope clusters. In aspects, T cell epitope clusters may contain significant numbers of putative T cell epitopes and EpiBars™ indicating a high potential for MHC binding and T cell reactivity.

Testing for cross-reactivity with host. The JanusMatrix system (EpiVax, Providence, R.I.) is useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in the therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score above 3.0 indicate high tolerogenicity potential and may be useful for Tregitope compositions of the present disclosure. In aspects, peptides with a JanusMatrix Homology Score below 2.0, below 2.5, or below 3.0 indicate low tolerogenicity potential and may be excluded from the Tregitope compositions of the present disclosure.

Methods for the Assessment of Tregitope Binding to Soluble MHC.

Synthesis of peptides. The Tregitopes of the present disclosure are produced by direct chemical synthesis or by recombinant methods (J Sambrook et al., Molecular Cloning: A Laboratory Manual, (2^{ED}, 1989), Cold Spring Harbor Laboratory Press, Cold Springs Harbor, N.Y. (Publ), herein incorporated by reference in its entirety). Sample Tregitopes are prepared using Fmoc-chemical (9-fluoronylmethoxycarbonyl synthesis, under the guidance and direction of the Inventors of the present invention at 21^{st} Century Biochemicals (Marlborough, Mass.). In certain aspects, the Tregitopes are capped with an n-terminal acetyl and c-terminal amino group. HPLC, mass spectrometry and UV scan (ensuring purity, mass and spectrum, respectively) analysis of the selected Tregitopes typically indicate >80% purity.

An amino acid analysis is conducted by a third-party contractor (New England Peptide, Inc., Gardner, Mass.) to confirm the predicted composition.

Mass Spectrum and Analytical HPLC analysis is performed by a second independent contractor (21^{St} Century Biochemicals, Inc., Marlboro, Mass.), further confirming the composition of the Tregitope.

HLA Binding Assay. Binding activity is analyzed at EpiVax (Providence, R.I.). The binding assay used (Steere A C et al., (2006), J Exp Med, 2003(4):961-71) yields an indirect measure of peptide-MHC affinity. For example, soluble HLA molecules are loaded onto a 96-well plate with the unlabeled experimental Tregitopes and labeled control peptide. Once the binding mixture reaches steady equilibrium (at 24 hours), the HLA-Tregitope complexes are captured on an ELISA plate coated with anti-human DR antibody and are detected with a Europium-linked probe for the label (PerkinElmer, Waltham, Mass.). Time-resolved fluorescence measuring bound labeled control peptide is assessed by a SpectraMax® M5 unit (Spectramax, Radnor, Pa.). Binding of experimental Tregitopes is expressed as the percent inhibition of the labeled control peptide (experimental fluorescence/control fluorescence multiplied by 100). The percent inhibition values for each experimental Tregitope (across a range of molar concentrations) is used to calculate the concentration at which it inhibits 50% of the labeled control Tregitope's specific binding, i.e., the Tregitope's IC₅₀.

Tregitopes are solvated in DMSO. The diluted Tregitopes are then be mixed with binding reagents in aqueous buffering solution, yielding a range of final concentrations from 100,000 nM down to 100 nM. Tregitopes are then be assayed against a panel of five common Class II HLA alleles: HLA-DRB1*0101, HLA-DRB1*0301, HLA-DRB1*0701, HLA-DRB1*1101, and HLA-DRB1*1501. From the percent inhibition of labeled control peptide at each concentration, IC₅₀ values are derived for each Tregitope/allele combination using linear regression analysis.

In this assay, Tregitopes are considered to bind with very high affinity if they inhibit 50% of control peptide binding at a concentration of 100 nM or less, high affinity if they inhibit 50% of control peptide binding at a concentration between 100 nM and 1,000 nM, and moderate affinity if they inhibit 50% of control peptide binding at a concentration between 1,000 nM and 10,000 nM. Low affinity peptides inhibit 50% of control peptide binding at concentrations between 10,000 nM and 100,000 nM. Peptides that fail to inhibit at least 50% of control peptide binding at any concentration below 100,000 nM and do not show a dose response are considered non-binders (NB).

### Example 1. Peptide Characterization by Binding to HLA Class II Molecules

Soluble MHC binding assays are performed on the Tregitopes of the present disclosure according to the methods described previously. IC₅₀ values (nM) are derived from a six-point inhibition curve.

EpiMatrix™ Predictions, calculated IC₅₀ values, and results classifications are reported for each Tregitope and HLA allele. Tregitope-allele combinations predicted to be cross-reactive between the host proteome are of particular note. Of these Tregitope-allele interactions, 90% will bind HLA, indicating that these Tregitopes generate measurable responses in human PBMC assays. Based on these findings, Tregitopes are selected for further testing.

Methods for Assessing the Phenotype of Peptide-Exposed APC

Surface expression of Class II HLA (HLA-DR) and CD86 by professional antigen presenting cells (APCs) is one way APCs modulate T cell response. Expression of Class II HLA surface marker is down-regulated in response to Tregitopes, and in particular to, the control Tregitope 167 (21^{st} Century Biochemicals, Marlboro, Mass.). Additionally, reduced expression of surface marker CD86 correlates positively with enhanced Treg function (Zheng Y et al., J Immunol, 2004, 172(5):2778-84). In this assay, candidate Tregitopes, including the selected Tregitopes, are tested for their ability to down-regulate the expression of Class II HLA and the co-stimulatory molecule CD86 on the surface of professional APCs, specifically dendritic cells.

Tregitopes are individually tested for regulatory potential using a proprietary APC phenotyping assay previously developed at EpiVax (EpiVax, Providence, R.I.). Previously harvested and frozen PBMC are thawed and suspended in chRPMI by conventional means. HLA typing is conducted on small, extracted samples of cellular material, provided to EpiVax, by Hartford Hospital (Hartford, Conn.). On assay day 0, 0.5×10⁶ cells are extracted, screened for the presence of surface marker CD1c (a marker specific to dendritic cells) and analyzed for the presence of surface markers HLA-DR and CD86 by flow cytometry. The remaining cells are plated (4.0×10⁶ cell per ml in chRPMI plus 800 ul media) and stimulated (50 □g/mL) with one of the four selected peptides or positive and negative controls including buffer only (negative control), Tregitope 167 (positive control) (21^{st} Century Biochemicals, Marlboro, Mass.), Flu-HA 306-318 (negative control) (21^{ST} Century Biochemicals, Marlboro, Mass.) and Ova 323-339 (negative control) (21^{st} Century Biochemicals, Marlboro, Mass.). Plated cells are incubated for seven days at 37° C. On assay day 7, incubated cells are screened by flow cytometry for the presence of surface marker CD11c. CD11c positive cells is then analyzed for the presence of surface markers HLA-DR and CD86. The experimental peptides are tested in samples drawn from five different human donors.

All whole blood samples used in the experiments are either sourced from healthy donors under IRB 07115 protocol (Clinical Partners, Johnston, R.I.), and leukocytes are isolated using a conventional Ficoll™ (GE Healthcare) separation gradient (Noble P B and Cutts J H, Can Vet J, 1967, 8(5):110-11), or Leukocyte Reduction Filters are obtained from the Rhode Island Blood Center (Providence, R.I.) to filter the white blood cells from whole blood obtained from healthy donors. After the whole blood is run through the filters, the filters are flushed in the opposite direction to push collected white blood cells out of the filter. The white blood cells are then isolated using a conventional ficoll separation gradient. The collected white blood cells are thereafter frozen for future use. When needed for use in an assay, the frozen white blood cells are thawed using conventional methods.

Exposure to putative Tregitopes on the phenotypes of dendritic cells is measured by multiple means. First, for each experimental condition, dot plots, contrasting surface expression of CD11c and HLA-DR, are produced. Dot plots of cells exposed to all control and experimental peptides are overlaid onto dot plots produced from control cells exposed to only the culture media. The overlay provides an effective method to visually observe shifts in HLA-DR distribution between Tregitope stimulated and unstimulated CD11c-high cells. Observed shifts in the distribution of HLA-DR is reported as a qualitative measure. Next, the change in intensity of HLA-DR expression for the CD11c-high segment of each dot plot is calculated as follows: percent change in intensity of HLA-DR expression equals Mean Florescence Index (MFI) of HLA-DR expression for peptide exposed cells minus MFI of HLA-DR expression for media exposed cells divided by MFI of HLA-DR expression for media exposed cells, times 100 (^{HLA-DR}MFI_{peptide}−^{HLA-DR}MFI_{media}/^{HLA-DR}MFI_{media}*100). The percent change for each peptide stimulant is compared. Next, the percent change in the percentage of HLA-DR-low cells present among the CD11c high population is calculated for each peptide relative to media control. Percent change in the percentage of HLA-DR-low cells is calculated, and equals the percent of HLA-DR-low for peptide exposed cells minus the percent of HLA-DR-low for media exposed cells divided by percent of HLA-DR-low for media exposed cells times 100 (^{HLA-DR-low}%_{peptide}−^{HLA-DR-low}%_{media}/^{HLA-DR-low}%_{media}*100). In this assay, a negative change is observed HLA-DR MFI and a positive change in percentage of HLA-DR-low cells present in the CD11c-high population indicates reduced expression of HLA and a shift to a regulatory APC phenotype. Data is used to compare the % of HLA+ and HLA− each peptide stimulant where the vehicle is media.

A similar process is used to assess the impact Tregitope exposure on surface expression of CD86, which is a costimulatory molecule known to promote T cell activation. First, for each experimental condition, dot plots contrasting surface expression of CD11c and CD86 are produced. Dot plots of cells exposed to all control and experimental Tregitopes are overlaid onto dots plots produced from control cells exposed to only the culture media. The overlay provides an effective method to visually observe shifts in CD86 distribution between Tregitope stimulated and un-stimulated CD11c-high cells. Observed shifts in the distribution of CD86 are reported as a qualitative measure. Next, the change in intensity of CD86-high expression for the CD11c-high segment of each dot plot is calculated. Percent change in intensity of CD86-high expression equals Mean Florescence Index (MFI) of CD86 expression for peptide exposed cells minus MFI of CD86-high expression for media exposed cells divided by MFI of CD86 expression for media exposed cells, times 100 (^{CD86-high}MFI_{peptide}−^{CD86-high}MFI_{media}/^{CD86-high}MFI_{media}*100). Next, the percent change in the percentage of CD86-low cells present among the CD11c high population is calculated. Percent change in the percentage of CD86-low cells equals the percent of CD86-low for peptide exposed cells minus the percent of CD86-low for media exposed cells divided by percent of CD86-low for media exposed cells, times 100 (^{CD86-low}% I_{peptide}−^{CD86-low}%_{media}/^{CD86-low}%_{media}*100). In this assay, a negative change is observed CD86 MFI and a positive change in percentage of CD86-low cells present in the CD11c-high population indicates reduced expression of CD86 and a shift to a regulatory APC phenotype.

### Example 2. Characterization of Peptide Exposed APC

Dendritic cell phenotyping assays are performed on select Tregitopes according to the methods described previously. Dot plots corresponding to each experimental condition tested in each of five human donors are prepared.

A series of dot plots is generated, representing the surface expression of CD11 vs HLA-DR analyzed on assay day 7 across the five donors in the presence of various peptide stimulants. Downward movement of the CD11c+/HLA-DR+ population is apparent in the samples treated with a potent Tregitope as compared to media control indicating an acquired regulatory phenotype. Tregitope 167 (positive control) and some of the other peptides will respond similarly, but the observed shift is more prominent with other more potent Tregitopes.

A series of dot plots is generated, representing the surface expression of CD11c vs CD86 analyzed on assay day 7 across the five donors in the presence of various peptide stimulants. An increase in CD86-low cells is present in the samples treated with a potent Tregitope, when compared to media control, and indicates a shift to the acquired regulatory phenotype. The dendritic cell phenotypic assays demonstrate that exposure to a potent Tregitope decreases expression of HLA-DR in all five subjects tested. Further, in four out of five subjects, exposure to a potent Tregitope increases the percent of CD86-low present among the CD11c-high cohort. Both trends indicate a shift towards an acquired regulatory phenotype.

Methods for Assessing Peptide Effects on Proliferation of Regulatory T cells

Previous studies performed by EpiVax (Providence, R.I.) demonstrated increased proliferation of regulatory T cells following exposure to known Tregitope including positive control Tregitope 167 (21^{st} Century Biochemicals, Marlboro, Mass.). In this assay, Tregitopes, including the Tregitopes of the instant disclosure, are tested for their ability to induce proliferation among CD4+CD25+ FoxP3+ regulatory T cells. Previously harvested and frozen PBMC are thawed and suspended in conditioned chRPMI (3.3×10⁶ cells/mL) by conventional means. Cells are stained with CFSE (Cat #: 65-0850-84, Affymetrix, Santa Clara, Calif.) and plated at 300,000 cells per well. Plates are incubated overnight (37° C. in 5% CO₂). On assay day 1, a Tregitope and a control peptide is reconstituted in sterile DMSO yielding a final stock concentration of 20 mg/mL. Previous titration experiments performed at EpiVax (EpiVax, Providence, R.I.) have established that stimulation with 0.5 μg/ml Tetanus Toxoid (TT) (Astarte Biologics, Bothell, Wash.) elicits a measurable CD4+ effector memory T cells response in PBMC drawn from healthy control donors (Rhode Island Blood Center, Providence, R.I.). Tetanus Toxoid stock (100 μg/mL) (Astarte Biologics, Bothell, Wash.) is diluted in conditioned chRPMI yielding a working concentration of 1 μg/mL. Plated cells are stimulated with either 100 μL of conditioned chRPMI (negative control), 100 μL Tetanus Toxoid solution (positive control) (Astarte Biologics, Bothell, Wash.), 100 μL of a dilution of 2991 μL Tetanus Toxoid solution plus 9 μL Tregitope solution, 100 μL of a dilution of 2997 μL Tetanus Toxoid solution plus 3 μL Tregitope solution, or 100 μL of a dilution of 6998.2 μL Tetanus Toxoid solution plus 1.8 μL Tregitope solution. In parallel, control wells with identical number of the same cells are incubated with control peptide solutions prepared as described for the Tregitope solutions. All plates are then be incubated for six additional days. On assay day five, 100 μL of supernatant is removed from each well and replaced with freshly conditioned chRPMI.

Highly activated regulatory T cells displaying elevated levels of FoxP3, CD25, Granzyme B and proliferation are selected. The gating strategy for highly activated regulatory T cells involves CD4+ T cells being gated for elevated CD25, Granzyme B, FoxP3, and low CFSE (proliferation). The results of the representative assay with no added TT is compared with the results of the representative assay with 0.5 μg/ml TT.

### Example 3. Tregitopes Strongly Induce a Population of Highly Proliferative, Activated Regulatory T Cells

Regulatory T cell proliferation assays are performed on the Tregitopes of the present disclosure according to the methods described previously. Gating on highly activated Granzyme B positive CD4⁺ T cells (CD25⁺ Granzyme B⁺) show that a subset of them are composed of highly proliferative regulatory T cells (CFSE low FoxP3⁺). A potent Tregitope increases the relative proportion of this population, while a control peptide has no significant effect. The increase in this population of highly activated, Granzyme B positive regulatory T cells closely correlates with the degree of effector T cell inhibition shown by different Tregitopes across multiple donors, markedly increasing in relative numbers only in those cases where the Tregitopes have an inhibitory effect on effector CD4⁺ cells. This is suggestive of the involvement of cytotoxic regulatory T cells in the inhibitory mechanism of potent Tregitopes. In total, this data demonstrates that a potent Tregitope strongly induces a population of highly proliferative, activated regulatory T cells rich in Granzyme B.

Methods for Assessing Peptide Effects on Proliferation of CD4+ Effector T Cells

CD4+ effector memory T cells contained within PBMC cell populations are induced to proliferate in response to stimulation with known T cell epitopes. A Tregitope binds multiple HLA molecules and can induce a regulatory phenotype in exposed APC (Clinical Partners, Johnston, R.I.). Results of the competitive inhibition HLA binding assay provides an indirect measure of peptide-MHC affinity (Steere A C et al., J Exp Med, (2006), 203(4):961-71, herein incorporated by reference in its entirety). Binding of experimental Tregitopes is expressed as the percent inhibition of the labeled control peptide (experimental fluorescence/control fluorescence multiplied by 100). The percent inhibition values for each experimental Tregitope (across a range of molar concentrations) are used to calculate the concentration at which it inhibits 50% of the labeled control peptide's specific binding. This value is referred to as the IC₅₀. HLA binding results and IC₅₀ for a Tregitope across five HLA-DR1 types indicates a high affinity of binding for the Tregitope across multiple HLA Class II types.

The purpose of this experiment is to establish the ability of a Tregitope to suppress the proliferation of antigen stimulated CD4+ effector memory T cells by either direct (engagement and activation of Treg) or indirect (modulation of APC phenotype) means. For the initial study, a control peptide is used as a negative control. In subsequent studies, the performance of a Tregitope is compared to the homologous peptide from which the Tregitope is derived.

Previously harvested and frozen PBMC are thawed and suspended in conditioned chRPMI (3.3×10⁶ cells/mL) by conventional means. Cells are stained with CFSE (Cat #: 65-0850-84, Affymetrix, Santa Clara, Calif.) and plated at 300,000 cells per well. Plates are incubated overnight (37° C. in 5% CO₂). On assay day 1, the Tregitope peptide and the control peptide are reconstituted in sterile DMSO yielding a final stock concentration of 20 mg/mL. Previous titration experiments performed at EpiVax (EpiVax, Providence, R.I.) have established that stimulation with 0.5 μg/ml Tetanus Toxoid (TT) (Astarte Biologics, Bothell, Wash.) elicits a measurable CD4+ effector memory T cells response in PBMC drawn from healthy control donors (Rhode Island Blood Center, Providence, R.I.). Tetanus Toxoid stock (100 μg/mL) (Astarte Biologics, Bothell, Wash.) is diluted in conditioned chRPMI, yielding a working concentration of 1 μg/mL. Plated cells are then stimulated with either 100 μL of conditioned chRPMI (negative control), 100 μL Tetanus Toxoid solution (positive control) (Astarte Biologics, Bothell, Wash.), 100 μL of a dilution of 2991 μL Tetanus Toxoid solution plus 9 μL Tregitope solution, 100 μL of a dilution of 2997 μL Tetanus Toxoid solution plus 3 μL Tregitope solution, or 100 μL of a dilution of 6998.2 μL Tetanus Toxoid solution plus 1.8 μL Tregitope solution. In parallel, control wells with identical number of the same cells are incubated with control peptide solutions prepared as described for the Tregitope solutions. All plates are then be incubated for six additional days. On assay day five, 100 μL of supernatant is removed from each well and replaced with freshly conditioned chRPMI.

On assay day seven, cells are removed from incubation. Cells are labeled for live/dead discrimination, for surface markers CD127, CCR7, CD4, CD45RA, and CD25 and for intracellular FoxP3. Stained cells are further prepared for FACS analysis by conventional means. Cells are first be gated to eliminate aggregates and dead cells. Live cells are gated for CD4 T cells and all subsequent analysis is done on this population. The activated Teffector population is identified as the CD4+/CD25-high/FoxP3-intermediate (CD4+/CD25^{hi}/FoxP3^{low}). In a parallel analysis of this identified T effector cell population, the proliferation of this major population is shown to correspond to a CD45RA-low and CCR7-low effector memory T cells. The major proliferations population correspond to the T effector memory phenotype (CD45RA-low/CCR7-low).

Proliferation of CD4+/CD25-high (CD4+/CD25^{hi}) T cells is estimated from the dilution of the CFSE stain (Cat #: 65-0850-84, Affymetrix, Santa Clara, Calif.) and % proliferation is determined by the CFSE-low (CFSE^{lo}) population.

### Example 4A. Tregitope Peptide Suppresses Proliferation and Activation of CD4+ Effector T Cells

The change in activation and proliferation of CD4+ effector cells when the proliferation stimulant (Tetanus Toxoid) is co-delivered with the Tregitope may be measured and the proliferative response of CD4+ T cells, comprised mainly of T effector memory cells, may be characterized.

T cell proliferation assays are performed on the Tregitopes of the present disclosure according to the methods described previously. Dot plots corresponding to each experimental condition tested for activation and proliferation are generated. Dot plots of the experimental condition tested for activation show that a Tregitope peptide strongly suppresses a population of activated effector CD4+ T cells (CD4+/CD25-high/FoxP3-intermediate, shown as CD4⁺/CD25^{hi}/FoxP3^{int}) reacting to Tetanus Toxoid in a dose-dependent manner, while control peptide have no appreciable effect. In the same experiment, the proliferation of total CD4+ T cells activated by a Tregitope (CFSE low cells) is strongly suppressed by the Tregitope peptide in a dose-dependent manner, while control peptide has little to no effect. Tetanus Toxoid stimulate a population of activated (CD25 high) CD4 T cells to proliferate (CFSE low), with approximately 90% of the activated cells also proliferating. This population of highly activated cells is actively suppressed by the Tregitope peptide in a dose-dependent manner, while the control peptide has no significant inhibitory effect on activated CD4 cells.

Further, gating on CD4+ and CD4− live cells demonstrate that the inhibitory effect of the Tregitope on cell proliferation is more pronounced on the CD4+ population than on the CD4− population. The Tregitope suppress proliferation of CD4− T cells in a dose-dependent manner. Negative control peptide show little inhibitory effect on the CD4− populations.

### Example 4B. Tregitope Peptide and its Homologous Peptide Suppress Proliferation of CD4+ Effector T Cells

Additionally, the inhibitory effects of the Tregitope with the homologous peptide from which the Tregitope is derived on the CD4+ T cell effector memory response to TT in PBMCs in normal donors are compared, analyzed and evaluated.

A study designed to evaluate the effect of the Tregitope peptide and the homologous peptide on the recall response to TT of PBMCs derived from two normal donors is undertaken. The protocol used is previously described. Tregitope concentrations range from 5, 10, 15, and 20 μg/ml. The Tregitope inhibit CD4+ T cell activation and proliferation of Teff responding to TT in a concentration-dependent manner. CD4+ T cell activation is reduced by 80-90% at 20 μg/ml (11 mM). The homologous peptide, when added with TT, inhibits both CD4+ T cell memory responses (CD4+ T cell activation and proliferation of Teff) in a concentration-dependent manner. CD4+ T cell activation is reduced by 50-70% at 20 μg/ml (11 mM). T cell populations respond with a 5-20% stronger inhibitor effect (for both CD4+ proliferation and Teff activation) for a given peptide concentration of either Tregitope peptide or homologous peptide across the range of concentrations tested. The similarity in peptide sequence between a Tregitope and its homolog, along with their parallel T cell inhibitory function, evidences that antigen-specific tolerance to the protein from which the Tregitope is derived is a useful method to stimulate tolerance to a homologous replacement protein. In this assay, both the Tregitope peptide and the homologous peptide suppress proliferation of CD4+ T cells and activate T effector T cells in a dose-dependent manner.

Methods for Assessing Tregitope Peptide Effects on CD8+ Effector T Cells.

CD8+ effector memory T cells contained within PBMC cell populations are induced to proliferate in response to stimulation with known class I T cell epitopes. A Tregitope binds multiple HLA alleles and induces a regulatory phenotype in exposed APC (Clinical Partners, Johnston, R.I.) (the gating strategy employed allows for the identification of the APC fraction in PBMC collected from the whole blood donors). The results of this assay establish the ability of a Tregitope to suppress the proliferation of antigen stimulated CD8+T effector memory T cells by either direct (engagement and activation of T_{Reg}) or indirect (modulation of APC phenotype) means.

T cell proliferation assays are performed on the Tregitopes of the present disclosure according to the methods described previously. PBMCs from two healthy donors are thawed and suspended in conditioned chRPMI (3.3×10⁶ cells/mL) by conventional means. Cells are stained with CFSE (Cat #: 65-0850-84, Affymetrix, Santa Clara, Calif.) and plated at 300,000 cells per well. Plates are incubated overnight (37° C. in 5% CO₂). On assay day 1, the Tregitope are re-constituted in sterile DMSO yielding a final stock concentration of 20 mg/mL. Intermediate solutions of Tregitope at twice the final concentration in chRPMI are prepared as described previously. Final concentration of Tregitope are tested from 2.5, 5, 10 and 20 μg/ml. As a CD8+ stimulating antigen, the CEF peptide pool, which consists of 23 MHC class I restricted viral epitopes derived from human cytomegalovirus, Epstein-Barr virus, and influenza virus, is used. CEF peptides are added to the wells (data shown for 2 μg/mL) with cells and media (control) or Tregitope at 0, 1, 2 or 4 μg/ml. All plates are incubated for six additional days. On assay day 5, 100 μL of supernatant is removed from each well and is replaced with freshly conditioned chRPMI.

Conventional methods are used to stain cells for live/dead marker, extracellular markers CD4, CD8□ and CD25, CD127, CD45RA and CCR7, and intracellular marker FoxP3. After FACS analysis, cells are gated to eliminate aggregates and dead cells. On the live cells population, CD8 □ and CD4 cells are gated separately and each population is analyzed for proliferation (CFSE low population) or activation (CD25-high/FoxP3 low/intermediate) as explained previously.

### Example 5. Tregitope Peptide Suppresses Proliferation of CD8+ Effector T Cells

The potential inhibition of CD8+ T cell response by a Tregitope peptide when PBMC from healthy donors are stimulated with CEF peptides mixture is tested. The Tregitope strongly inhibit the CD8+ T cell proliferative response to CEF peptides, as well as activation of CD8+ cells. The percent of proliferating CD8+ T cells (CFSE low) and the percent of activated CD8+T effector cells (CD25^{hi} FoxP3^{int/low}), decreases with increasing concentrations of the Tregitope, demonstrating that Tregitope also have an inhibitory effect on the CD8+ T cell population. In both cases, the Tregitope strongly inhibit the response in a dose-dependent manner.

(7) Generation of Tregitope-Blood Component Conjugates

Linkage of a Tregitope with a blood component conjugate, such as albumin, is useful as a carrier protein for Tregitope payload. Tregitope-blood component conjugates as disclosed herein can extend the half-life of Tregitopes in vivo, protect Tregitopes from rapid proteolytic degradation, protect Tregitopes from rapid clearance from circulation and/or rapid kidney excretion, allow for wide distribution of Tregitope-blood component conjugates throughout the body of a subject, aid in delivery of Tregitopes to appropriate immune cells (such as macrophages and APCs), allow the Tregitopes to be processed by the endocytic pathway of certain immune cells (such as macrophages and APCs), and/or aid in the presentation of Tregitopes as an antigen by said immune cells.

Tregitope-blood component conjugates are formed by modifying a Tregitope peptide by attaching a reactive moiety to the Tregitope peptide to create a modified Tregitope peptide, then forming a bond between reactive moiety of the modified Tregitope peptide with a reactive functionality on a blood component, as disclosed in U.S. Pat. Nos. 6,849,714, 6,887,470, 7,256,253, and 7,307,148. Albumin is a preferred blood component because it contains an Fc neonatal binding domain that will carry the Tregitope-albumin conjugate into the appropriate cells, such as macrophages and APCs. Further, albumin contains a cysteine at amino acid 34 (Cys³⁴) (the location of the amino acid in the amino acid sequence of human serine albumin), containing a free thiol with a pKa of approximately 5, which may serve as a preferred reactive functionality of albumin. Cys³⁴ of albumin is capable of forming a stable thioester bond with maleimidopropionamido (MPA), which is a preferred reactive moiety of a modified Tregitope peptide. The stable thioester bond between albumin and the Tregitope peptide modified with MPA cannot be cleaved under physiological conditions.

The Tregitope peptide is preferably selected from SEQ. ID NOS: 1-55. One or more lysines may be present on the N-terminus of the Tregitope peptide, such as added onto to the N-terminus of peptides selected from SEQ. ID NOS: 1-55. A linker, such as a polyethylene glycol linker (e.g., PEG2 or PEG12), is present between the one or more lysines and the Tregitope sequence, or at the N-terminus of a Tregitope sequence. In aspects, a lysosomal cleavage site, such as a Cathepsin B site, optionally consisting (sequentially from N-terminus to C-terminus) of valine and citrulline, is present between the PEG2 moiety and the Tregitope sequence, and/or between one or more Tregitopes. In aspects, one or more Tregitopes may be present on the construct, optionally more proximate to the C-terminus than the linker. In aspects, one or more lysosomal cleavage sites are present between multiple Tregitopes (for example, such that a single lysosomal cleavage site separates two Tregitopes, or such that one lysosomal cleavage site is present between a first and second Tregitope, and another lysosomal cleavage site is present between a second and third Tregitope, and so on). In aspects, one or more antigen peptides associated with immunogenicity in diabetes (“T1Dgen” peptides; e.g., PPI-derived peptides) may be present on the construct, optionally separated from one or more Tregitopes by a lysosomal cleavage site. In aspects, one or more lysosomal cleavages sites are present between multiple T1Dgen peptides (for example, such that a single lysosomal cleavage site separates two T1Dgen peptides, or such that one lysosomal cleavage site is present between a first and second T1Dgen peptides, and another lysosomal cleavage site is present between a second and third T1Dgen peptides, and so on). A maleimide-based chemistry may be used to covalently link the modified Tregitope peptide to a blood component, preferably serum albumin, in a 1:1 molar ratio. Linking the modified Tregitope peptide to a blood component may be performed in vivo or ex vivo.

Cathepsin B is the first described member of the family of lysosomal cysteine proteases. Cathepsin B possesses both endopeptidase and exopeptidase activities, in the latter case acting as a peptidyldipeptidase. Cathepsin B may be included in the Tregitope peptide design to facilitate the proper cleavage of the Tregitope from Albumin once it is in the lysosomal compartment in the antigen presenting cells. The Valine-Citrulline is a Cathepsin B cleavage site that has been previously used successfully and has been FDA approved in Antibody Drug conjugate (e.g., monomethyl auristatin E (MMAE) conjugate in the drug brentuximab vedotin). Our interest in incorporating the site is to provide cleavage sites that would allow the proper cleavage of the Tregitope from the human serum albumin for efficient MHC class II presentation once it is in the APC.

### Example 6. Generation of a Tregitope-Albumin Conjugate by Ex Vivo Conjugation

Standard Fmoc (9-fluorenylmethoxycarbonyl) solid-phase peptide synthesis chemistry is used for peptide synthesis. Synthesis is performed on Intavis™ MultiPep™ automated peptide synthesizers. Amino acids are added stepwise to the growing peptide chain (C-terminus to N-terminus; right to left), while attached to an insoluble polystyrene resin support. Amino acid building blocks, protected at their amino terminus by an Fmoc group, are coupled to the growing chain after activation of the carboxylic acid terminus via one or more condensation reagents (e.g., Hexafluorophosphate Azabenzotriazole Tetramethyl Uronium (HATU), O-(1H-6-Chlorobenzotriazole-1-yl)-1,1,3,3-tetramethyluronium hexafluorophosphate (HCTU)). The reaction by-products at each addition are removed by solvent washing (6×, Dimethylformamide (DMF)). Following each coupling and capping step, the Fmoc is removed via piperidine deprotection of the peptide resin (performed 2×; 20% in DMF volume/volume with 0.1M HOBt to suppress Asp dehydration), the resin washed with DMF 6×, and the next amino acid added. A Cathepsin B cleavage site is incorporated at the N-terminus of the Tregitope sequence.

For a PEG2 construct (“PEG2” or “P2”), after the desired Tregitope peptide is completed a PEG2 moiety is added to the N-terminus, followed by the addition of 4 lysines to the N-terminus. The PEG2 and Lysines are incorporated to provide a potential docking area for the cathepsin B. Additionally, the PEG2 and lysines (via the primary amine on the lysine side-chain) will increase the solubility of the final construct. The composition of the PEG2 construct is shown in Table 1 (below).

TABLE 1

PEG2 construct composition
HSA (Cys 34)- Maleimide linkage- KKKK-Peg2-
Val-Cit (CatB cleavage site)_Tregitope_Nle

For a PEG12 construct (“PEG12” or “P12”), two additions of a PEG6 are added after the Tregitope peptide synthesis. In this case, no lysines are added. Increasing the PEG length also provides a docking region for Cathepsin B and improves the solubility of the Tregitope. The composition of the PEG2 construct is shown Table 2 (below).

TABLE 2
PEG12 construct composition
HSA (Cys 34)-Maleimide linkage-Peg12-Val-Cit
(CatB cleavage site)_Tregitope_Nle

Subsequently, a small amount of the peptide constructs are removed from the resin and the peptide sample cleaved and deprotected by treatment with trifluoroacetic acid (TFA. 92.5% v/v) in the presence of TIS (triisopropylsilane, 5%) and water (2.5%) to scavenge side-chain protecting groups. Each crude, linear, peptide (˜3-5 mg) is purified by preparative reversed phased HPLC (Gilson) using a 20 mm×50 mm YMC C18, 5 μm, Hydrosphere column. The peptides are purified to >90% purity (determined via analytical HPLC) and the mass verified utilizing an ABI-SCIEX QSTAR XL Pro Qo-TOF mass spectrometer prior to the Cathepsin B evaluation. The remaining peptides (PEG2-Tregitope and PEG12-Tregitope) are left on the resin for the addition of 3-maleimidoproprionic acid (MPA) at a later time.

Recombinant human Cathepsin B (catalog 953-CY of R&D Systems™) is used to evaluate the cleavage of the Val-Cit site engineered into the Tregitope peptide. The activity assay protocol is used according to the R&D Systems™'s recommendations with final assay conditions of 0.01 μg rhCathepsin B and 10 μM of peptide substrate. After incubation of Cathepsin B with purified peptides (at RT for 15 min, the peptide is evaluated by mass spec using the Qstar XL Pro™. It is determined that the PEG2 peptide did not have successful cleavage, and further modification of the Cathepsin B protocol did not produce successful cleavage. For the PEG12 product, successful cleavage is demonstrated.

After evaluation of the cleavage of the Val-Cit site by Cathepsin B, the reactive moiety of 3-maleimidoproprionic acid (MPA) is added to the N-terminus of the PEG2 and PEG12 peptides. Similar, to the amino acid building blocks, the MPA is protected by an Fmoc group, and coupled to the growing chain after activation of the carboxylic acid terminus. The final MPA-Tregitope constructs is removed from the resin and the peptide sample is cleaved and deprotected by treatment with trifluoroacetic acid (TFA. 92.5% v/v) in the presence of TIS (triisopropylsilane, 5%) and water (2.5%). Each crude, linear, peptide (˜20 mg) is purified by preparative reversed phased HPLC (Gilson™) using a 20 mm×50 mm YMC C18, 5 μm, Hydrosphere column. The MPA-peptides are purified to >90% purity (determined via analytical HPLC) and the mass verified utilizing an ABI-SCIEX QSTAR XL Pro™ Qo-TOF mass spectrometer (data not shown). A total of 15 mg of the MPA-P2 and MPA-P12 Tregitopes is used in the subsequent conjugation to rHSA (Albucult-Novozyme™) to construct the final preformed HSA-Tregitope conjugate.

Ellman's Reagent (5,5′-dithio-bis-[2-nitrobenzoic acid]) is used to estimate sulfhydryl groups in a sample by comparing to a standard curve of a sulfhydryl-containing compound such as cysteine. Ellman's test is performed on rHSA (Sigma™, Albucult®) at multiple concentrations to ensure the accuracy of the analysis. Ellman's reagent (Sigma™) rHSA from Sigma™ lot RF-009 is evaluated for free cysteine that would be available for conjugation with the maleimide. We estimate that 78% of the rHSA had free cysteine available, as shown in Table 3 (below).

TABLE 3
Estimation of free cysteine in rHSA samples

| | | | | | Moles | |
|---|---|---|---|---|---|---|
| | | | | | huHSA per | |
| Grams | Moles | O.D. | | | mole free | % free |
| rHSA | huHSA | 412 | Concentration | Moles | cysteine | cysteine |
| 0.001 | 1.50376E−08 | 0.059 | 4.1696E−06 | 1.16749E−08 | 1.29 | 77.64 |
| 0.002 | 3.00752E−08 | 0.12 | 8.4806E−06 | 2.37456E−08 | 1.27 | 78.95 |

Peptide is solubilized in dH20, rHSA added (15 mg/ml) and 100 mM Phosphate buffer added to give a final pH of 8. The peptide is added in a 10× molar excess to the HSA. Peptide/HSA is incubated at room temperature for 2 h followed by incubation at 4° C. for approximately 24-30 hours.

After the conjugation step, the HSA-conjugate is then dialyzed into PBS (pH 7.0) first at room temperature for 2 hours, followed by 2 changes to fresh PBS at 4° C. for 18-24 h. This process removes excess peptide from the HSA and HSA-Tregitope conjugate preparation.

The Ellman's test is performed on each conjugate to demonstrate conjugation of the peptide via the rHSA free Cysteine, and determine the efficiency of conjugation in the reaction. The HSA-conjugation preparation does note remove the reduced HSA (mercaptabumin), inherent in the preexisting preparation (˜22% of the HAS pre-conjugation). The remaining unreacted HSA is determined to be 14% for the HSA-MPA_P2-Tregitope construct, meaning after conjugation with the maleimide-Tregitope 14% of the free cysteine remains. Thus, ˜64% of total rHSA preparation is reacted with the MPA_P2-Tregitope peptide.

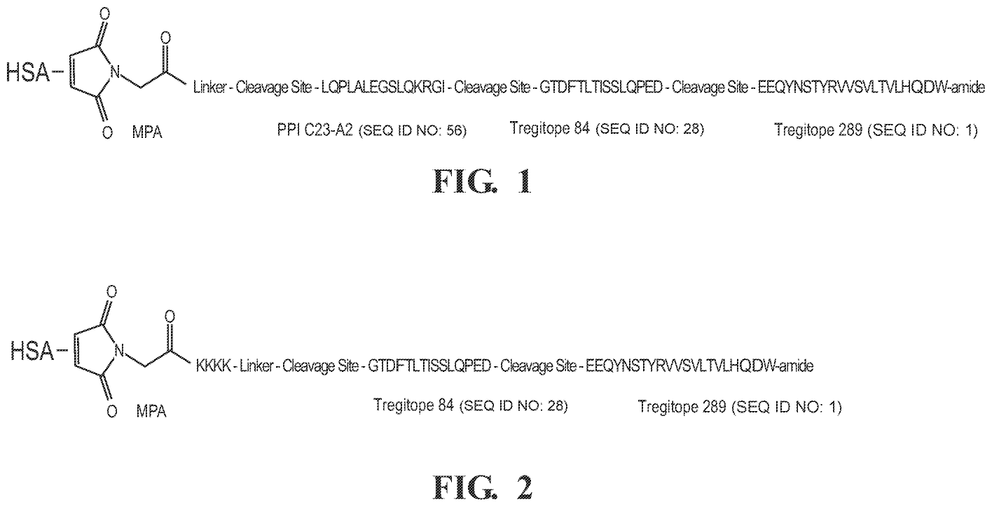

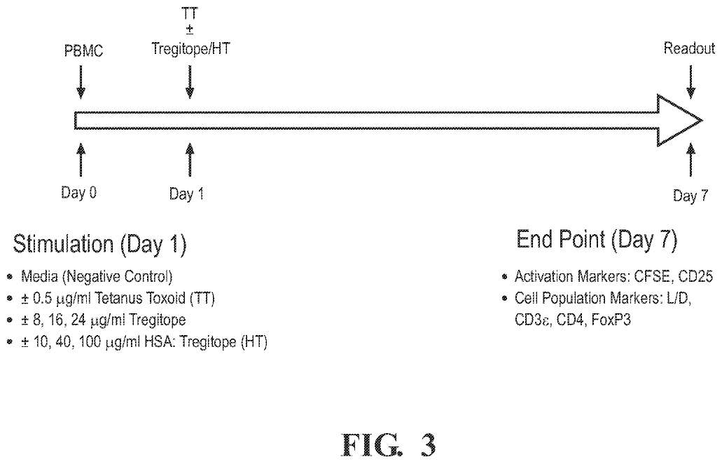

Additional Tregitope-albumin conjugates include those of FIGS. 1 and 2.

(8) Methods for Assessing Effect of Tregitope-Blood Component Conjugates on Immune Cells

A maleimide-based chemistry may be used to covalently link a Tregitope payload to recombinant HAS (rHSA) in a 1:1 stoichiometry. Maleimido-propionamido (MPA) forms a stable thiol ester conjugate with the available free Cys34 in HSA. HSA leverages the neonatal receptor (FcRn) recycling pathway, increasing the half-life of any conjugated payload, and potentially decreasing the need for repeat dosing. rHSA is also known to deliver conjugated payloads to the lymph nodes and is endocytosed by dendritic cells and other antigen presenting cells that express FcRn.

EpiVax designed an rHSA-Tregitope conjugate to contain cleavage sites between the Tregitopes. The cleavage sites are specific for an early endosomal protease, which enable the Tregitopes to be liberated from the rHSA molecule, increasing the efficiency of MHC class II presentation on the cell surface. The long and substantiated history of this FDA-Approved rHSA conjugation chemistry approach, as well as its successful manufacturing history support its selection for delivery of our T1D payload.

Once Tregitope-blood component conjugates are formed, for example as described in Example 6 and subsection 7 of the examples section, as well as the detailed description, the Tregitope-blood component conjugates are evaluated for their effectiveness in inhibiting effector T-cells and activating regulatory T-cells and their proliferation, for example in comparison with Tregitope peptides alone. Further, the Tregitope-blood component conjugates are evaluated for their capacity to induce immune tolerance against certain antigens

### Example 7. Evaluation of the Inhibitory Effect of Tregitope-Albumin Delivery Vehicle

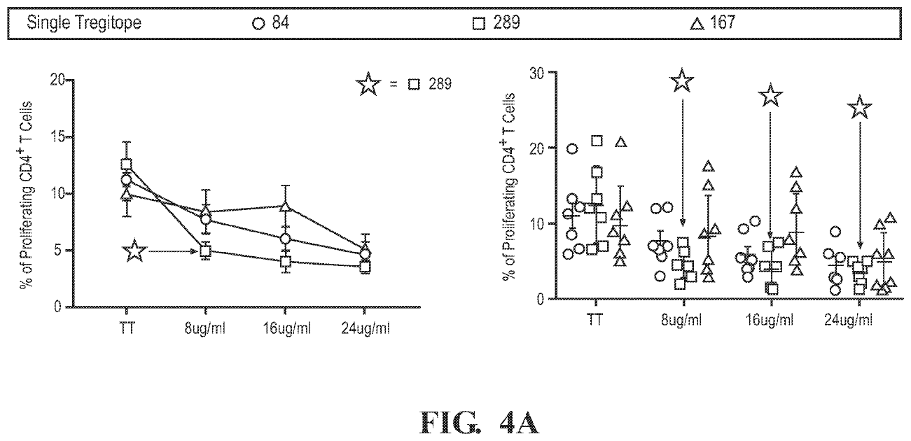

To determine the inhibitory effect of the Tregitope delivery vehicle, healthy donor PBMCs are used in a tetanus toxoid bystander suppression assay (TTBSA), and analysis is done on CD4 T-cell proliferation, activation of T cells, frequencies of T effector and T regulatory cells to determine the ratio of Treg/Teff, as is displayed in FIG. 3.

So as to optimize the best combination of Tregitopes for translation to the clinic, the effect of combinations of Tregitopes for their ability to synergistically suppress effector T-cell responses in vitro is analyzed. To facilitate these comparisons, a high throughput in vitro assay was developed using human donor peripheral blood mononuclear cells (PBMCs). This assay, referred to as the Tetanus Toxoid Bystander Suppression Assay (TTBSA), takes advantage of the ability of Tregs to suppress T memory cells specific to Tetanus that are elicited in individuals with a history of Tetanus toxoid (TT) vaccination.

At day 0, PBMCs are incubated and stained with Carboxyfluorescein succinimidyl ester (CFSE) dye. At day 1, cells are stimulated with by adding media, Tetanus Toxoid, and either: 8, 6, or 24 μg/mL of a Tregitope; or 10, 40, or 100 μg/mL of a Tregitope-albumin conjugate. Tetanus Toxoid is used at a final concentration of 0.5 μg/ml, where the concentration is methodically titrated and optimized to measure the inhibitory capacity of Tregitopes. Negative controls, including media-only, are included. At day 7, L/D cell population marker, extracellular stain, and intracellular stain are added to the cells. At day 8, a readout is taken. Cell sorting assays for analysis of activation markets (e.g., CFSE, CD25) and cell population markets (e.g., L/D, CD2c, CD4, and FoxP3) are performed.

Incubation of donor PBMCs with TT stimulates expansion of T effector cells. Tregitopes are added to PBMC in vitro with TT, and activate CD25^{hi}FoxP3^{hi} regulatory T cells suppressing expansion of TT-specific T effector cells. Tregitopes significantly inhibit the proliferation (as is measured by CFSE dilution) and activation (as is measured by CD25 expression) of CD4+T effector cells in a dose dependent manner, and also slightly expand Tregs (CD25⁺/FoxP3⁺/CD127^{lo}), which is suggested by an increase in the ratio of Treg/Teff cells. A reduction of effector T cell proliferation is a direct consequence of the activation of T regulatory cells and/or the conversion of TT-specific T effector to Treg, for example as is supported by the induction of Treg in vivo.

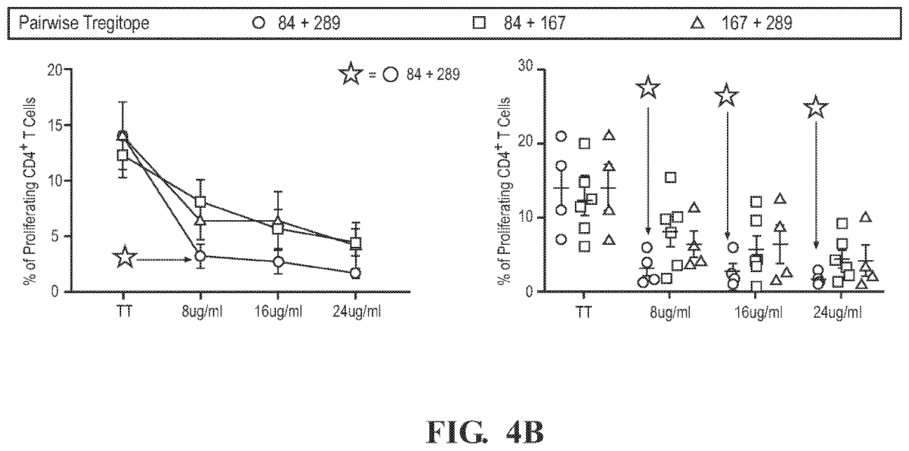

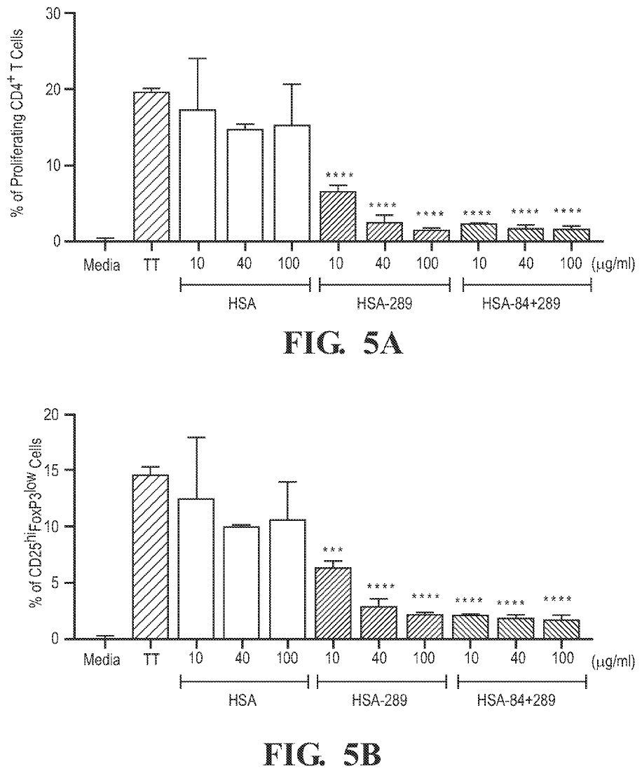

Using the TTBSA, each of a number of available Tregitopes individually and in pairwise combinations is examined for their potential to suppress CD4+ T cell proliferation. The most promising IgG-Tregitope peptides are selected for further testing. A certain Tregitope, Tregitope 289 (SEQ ID NO: 1), is the single Tregitope has the most suppressive activity in the TTBSA as compared to the other single Tregitopes. Combining Tregitope 289 with Tregitope 084 (SEQ ID NO: 28), an even greater suppressive effect on TT-specific T cell proliferation is observed (see FIGS. 4A and B). Conjugating 289+084 to rHSA improves their efficacy in vitro (see FIGS. 5A and B).

Thus, using TTBSA, it is shown that HSA-Tregitope conjugates inhibit CD 4 T-cell proliferation and activation, and increase the ratio of Treg cells to Teff cells.

### Example 8. Evaluation of the Effectiveness of Preformed Conjugate HSA-Tregitope Therapeutics and Maleimide-Tregitope Peptide Therapeutics

The effect on the response to OVA immunization of preformed conjugate HSA-Tregitope conjugates and a free-maleimide-Tregitope peptide is evaluated. The latter free-maleimide peptide forms a conjugation in vivo after injection via the reactive maleimide group to the free-Cys34 of the subject's endogenous HSA. 5 mgs of the MPA-P2 and MPA-P12 is used as free-MPA-Tregitope, with the unconjugated HSA in the sample being accounted for by calculating the molar ratio of conjugated to unconjugated HSA.

Mice (female C57BL/6) are immunized s.c. with 50 mg ovalbumin (OVA) on day 0 (CFA) and day 14 (IFA). The preformed HSA conjugate treatments is administered with the OVA in CFA on day 0. Test groups include OVA/HSA-P2-high and OCA/HSA-P2-low. Per injection OVA is 50 μg, and HSA at 800 μg, and HSA-P2H (high) conjugation is at 825 μg (˜20 μg Tregitope). HSA-P2L (low) conjugation is at 100 μg (˜3.7 μg Tregitope). Four control groups include PBS only, PBS/OVA, HSA/OVA, and Tregitope/OVA. A last arm is included to evaluate the utility of the free-maleimide Tregitope peptide and is administered by IV into tail vein. There are five mice per group.

Mice are sacrificed on Day 17. Upon sacrifice, cardiac bleeds and spleens are harvested for each animal. IFNγ/IL2 fluorospot assays, IFNγ/IL17 fluorospot assays, CD4 T cell proliferation, and T cell characterization are performed on the splenocytes stimulated with OVA. PHA is used as a positive control stimulation for spleen cell assays. All of the wells in PHA stimulation are confluent. An acceptance criteria is used wherein SFC (spot forming cells) after stimulation must be greater than 50 spots/10⁶ over negative control (media wells) and must also have a stimulation index greater than 2. According to both the IFNγ/IL2 fluorospot and IFNγ/IL17 fluorospot assays, IFNγ production is inhibited by treatment, and the HSA-only control group is inhibited less compared than the treatment groups.

For T-cell proliferation and characterization assays, splenocyte samples are evaluated for induction of FoxP3 expression in TCR Tg cells and for the suppression of OVA specific T cell proliferation (in response to OVA peptide in vitro) by CFSE dilution. To detect FoxP3⁺ Tregs, a single-cell suspension of draining lymph nodes is incubated with 2.4G2 mAb (anti-CD16/32, ATCC) for 15 minutes to block FcR then is stained with anti CD3, CD4, CD25 and anti-clonotypic KJ1-26 for 40 minutes at 4° C. KJ1-26 is specific for clonotypic TCR expressed by DO11.10 transgenic mice. Cells are then be permeabilized and stained for FoxP3 nuclear expression and acquired on a Thermo Attune NxT Autosampler™ for FACS analysis. The CD4⁺CD25⁺FoxP3⁺KJ1-26⁺ live cell gate population is established to determine the number and proportion of OVA-Specific T regulatory cells compared to PBS or HSA alone.

Antigen-specific T cell proliferation is evaluated by CFSE dilution. Draining lymph nodes are harvested, are stained with cell proliferation dye CFSE, and a single-cell suspension is prepared at 2×10⁶ cells/mL. Cells are added to 96-well plates at 100 μL per well in the presence of 10 μg/ml concentration of OVA 323-339 (New England Peptide, Gardner, Mass., USA). Cells are stimulated for 72 hours and harvested for staining with CD3a, CD4, CD8, CD54RA, CCR7, CD25, CD127, IFNγ HLA-II, CD69, CD154, IL-17, IL-21 for 40 minutes at 4° C. Cells are be fixed, permeabilized and stained for FoxP3 expression and analyzed by flow cytometry. An increase of OVA-specific KJ1-26⁺CD4⁺CD25⁺FoxP3⁺ adaptive (converted) T regulatory cells in mice treated with free maleimide-Tregitopes and HSA-Tregitope conjugates as compared to mice treated with rHSA is observed. Free maleimide-Tregitopes and HSA-Tregitope conjugates more effectively reduces OVA-specific proliferation of KJ1-26⁺ CD4⁺ T effector cells as compared to rHSA alone.

Anti-OVA antibodies in serum from the bleeds harvested on day 17 are evaluated in serum by ELISA, including a serial dilution plot and a standard ELISA to determine antibody concentrations. Mice treated with HSA-conjugates and free maleimide have lower serum antibody titers compared to no treatment, as indicated by absorbance at different dilutions, as well as comparison of absorbance over a standard curve.

(9) Methods for Assessing the Efficacy of Tregitope-Blood Component Conjugates in the Prevention and Treatment of Type 1 Diabetes

The demonstration that Tregitopes induce antigen-specific tolerance to islet antigens will have a radical impact on the field of diabetes therapy, potentially abrogating the need for insulin therapy in T1D; additional applications of HT-T1D may include preserving function after islet transplant.

Tregitopes, found in the Fc and framework of the variable domain of IgG, are a set of six natural Treg epitopes that bind to multiple MHC class II molecules and suppress inflammatory responses to co-administered antigens (Ag) in vitro and in vivo. Co-delivery of Tregitopes with target Ag is key to Ag-specificity. Tregitopes contained in high dose intravenous IgG therapy (IVIG) may activate CD4+CD25+FoxP3+ regulatory T cells, as seen in humans and in mice. Tregitopes may explain (in part) the effect of IVIG, which is a widely used therapy for treatment of autoimmune diseases. The mechanism of action of Tregitopes has been validated in eight independent laboratories and a wide range of models (including Scott et al. (AI disease models), Najafian et al. (Transplant), Khoury and Elyaman (Multiple Sclerosis), Mingozzi and Hui, UniQure (Inflammatory Bowel Disease), Moingeon et al. (Allergy)).

Early work by EpiVax on Tregitopes pointed to induction of antigen-specific tolerance to co-administered antigens. Published studies show that Tregitope-specific natural Tregs (nTregs) appear to modulate T effector responses by inhibiting the activity of autoreactive effectors and/or by changing the phenotype of Teff to aTregs. Based on this data, the inventors hypothesized that when Tregitopes are co-processed and co-presented with target Ag by the same APC, they activate a subset of circulating nTregs and induce aTregs. Most recently, Treg transfer experiments in allergy models have recently reconfirmed the antigen-specificity and efficacy of Tregitope treatment.

Tregitopes may provide a unifying mechanism for disparate IVIG effects in murine studies. The anti-inflammatory activity of IVIG has been attributed to Fc-gamma receptors, blockade of the neonatal Fc receptor (FcRn), or interaction with novel cell-surface receptors DC-SIGN and DCIR. However, Tregitope induction of regulatory T cells provides a better explanation for the transformation of T cell phenotype with IgG and the increase in aTregs after IVIG treatment in skin transplants and in EAE. Tregitopes also provide a new mechanism of action for IVIG in human autoimmune diseases. Engagement of Tregs by Tregitopes in IVIG may elucidate reports that IVIG induces Treg expansion in vivo and that IgG-derived peptides (similar to Tregitopes) have immunosuppressive effects.

Tregitopes have also significantly transformed the field of monoclonal antibody (mAb) development. The presence of Tregitopes in mAbs is associated with reduced immunogenicity in the clinic. Tregitopes have been eluted from mAb-pulsed Ag-presenting cells. EpiVax has developed risk-assessment tools that predict clinical immunogenicity of mAbs based on Tregitope-adjusted scores.

HSA-Tregitope (HT) may transform the treatment of numerous autoimmune diseases. Recombinant rHSA-Tregitope drug products can be produced in large scale under GMP at Ajinomoto (San Diego, Calif.).

The inventors hypothesize that conjugating two highly effective Tregitopes to a recombinant albumin delivery vehicle with a T1D target antigen (HT-T1D) will induce more effective T1D antigen-specific tolerance than PPI antigen alone and will have the potential to induce islet cell antigen-specific tolerance, arresting islet cell destruction in a murine model of T1D.

The HT-T1D therapy described in Example 9, below, Tregitopes SEQ ID NO: 1 and SEQ ID NO: 28 (as exemplary Tregitopes of the instant disclosure) are chemically linked to rHSA with pre-proinsulin peptide (PPI) SEQ ID NO: 56. The Tregitope-containing therapy is administered prior to onset of diabetes for dose ranging studies and administered after onset of diabetes for POC. Well-established methods are applied for validating the efficacy of HT-T1D treatments in vivo murine models, scale up production, and evaluate safety and toxicity of the drug product in GLP conditions under the guidance of experienced immunologists and drug development experts, to aid in moving to treatment and prevention to humans.

### Example 9. Antigen-Specific Tolerance Due to Co-Presentation with rHSA-Tregitope Fusion Protein In Vivo

Briefly, recombinant rHSA-Tregitope conjugate protein containing two Tregitopes is administered in conjunction with PPI peptides to NOD mice during the onset of diabetes, to test the impact of the combined therapy on the progression of diabetes. Diabetic animals will be treated after confirmation of blood glucose of 200-350 mg/dl is obtained. Mice treated with rHSA with the two Tregitopes+PPI are expected to significantly more able to control blood glucose over time in comparison to untreated or rHSA-treated control group. Between days 31 and 49, mean blood glucose is expected to be significantly lower in the rHSA with the two Tregitopes+PPI group as compared to rHSA alone. Severe diabetes (BG>600) or death is expected to occur at lower frequency or with a significant delay in the combined Tregitope-fusion-PPI drug-treatment group peptides. Statistical differences between the absolute BG levels between groups is expected to have a p-value of less than 0.05. The study demonstrates that rHSA is an effective delivery vehicle for Tregitopes and that, when co-administrated with PPI peptides, the HT-T1D combination reduces blood glucose out to >7 weeks in NODs.

### Example 10. Identification of an Optimal Dose and Treatment Regimen for Testing HT+T1D Target Antigen (PPI) in an In Vivo Model for T1D

The dose of HT-T1D (containing rHSA) that is tolerated by NOD mice is determined. Additionally, a determination is made of which dose is able to prevent the onset of diabetes in a two-dose regimen, in the well-characterized NOD murine model of spontaneous autoimmune diabetes. While NOD mice are intolerant to Human albumin, when Tregitope was fused with rHSA in a previous study, it was well tolerated in previous studies and significantly prevented diabetes development in both preventative and therapeutic studies of T1D.

First, testing is done for tolerance and initial efficacy of the HT and HT-T1D conjugates in a four arm pilot preventive study in NODs, lasting approximately 20-30 weeks. At 8 weeks, NOD mice (female; 8 mice/group) are divided into four treatment groups each of which receive two split day injections of the control rHSA, HT (including Tregitopes SEQ ID NOS: 1 and 28), and HT-T1D (including PPI peptide SEQ ID NO: 56) on Days 0/1 and 14/15. Tregitopes are delivered at 100 μg each (200 μg total Tregitopes) for both the HT and HT-T1D. rHSA is dosed at equivalent amounts for the rHSA control group. Mice are followed until 30 weeks for the development of diabetes. Cage-side observations are on a daily basis, and non-fasting blood glucose, clinical status and weights are assessed twice weekly. Mice are euthanized if their BG stays above 600 consistently (BG≥600 5 times), or if they lose excessive body weight (>20% of initial body weight), or if they appear moribund. Treatment groups include no treatment and rHSA controls. The repeat injection of HT and HT-T1D is expected to be tolerated without any significant side effect due to the presence of the Treg epitopes, and both products are expected to control diabetes development as determined by the number of mice that have BG level lower than 250 mg/dL at the end of 30 weeks.

Next, testing is done on multiple treatments of lower (25 μg/Tregitope; 50 μg total) and higher dose (100 μg/Tregitope; 200 μg total) of HT and HT-T1D in the same well-established preventative T1D model in NOD mice. However, the parameters of this study are modified in accordance with the results from tolerance and initial efficacy studies (e.g. adding additional doses, or the high dose may not be well tolerated in NODs, which are known to be reactive to rHSA). Starting at 8 weeks of age, NOD mice (female; 16 mice/group) are divided into eight treatment groups each of which receive four split-day injections on Days 0/1, 14/15, 28/29 and 42/43. Each group is treated as indicated below and followed over 20 weeks for diabetes development (or longer). Tregitope-containing conjugates are administered so that the lowest dose contains at least 25 μg of each Tregitope peptide. rHSA is given as a negative control treatment. Blood glucose (BG) levels are measured and recorded twice weekly for up to 20 weeks; the mice are considered to be diabetic if their BG level is ≥250 mg/dL for two consecutive measurements. rHSA-PPI A23-C2 is compared to HT-T1D. In the preventative study, without treatment at least 50% or up to 80% of NOD mice develop diabetes by 20 weeks. Treatment with HT-T1D using at 16 mice per group, there is expected to be a statistical 80% power to observe a 50% difference (from 60% to 30% penetrance) in the NOD T1D prevention model. Treatment with HT (rHSA-Tregitope) is expected to reduce the incidence of diabetes in NOD mice as compared to PBS or rHSA control injected mice. Combining the T1D-disease antigen PPI peptide to rHSA-Tregitope conjugate (HT-T1D) is expected to significantly prevent the progression of diabetes, controlling BG level and insulitis development.

Overall, the rHSA-conjugated Tregitopes with PPI peptide SEQ ID NO: 56 (HT-T1D) are expected to significantly prevent diabetes development in preventative study in NOD mice at a dose and regimen that is scalable.

### Example 11. Scaling Up of HT-T1D Conjugate and Characterization for Therapeutic Studies

Large-scale manufacturing and characterization of the research-grade HT-T1D will be performed. Characterization of the conjugate will include HPLC purification, evaluation of conjugation percentage, and in vitro peptide cleavage validation by HPLC. Demonstration will be done of HT-T1D efficacy in both the adaptive tolerance induction in DO11.10 mice, as well as the prevention of T1D in NOD mouse model. Successful production of 50 g of GMP grade HT-T1D will occur. There will be 50 g production of GMP grade HT-T1D.

### Example 12. Demonstration of the Effect of HT-T1D Conjugate in a Therapeutic Model of Human T1D

The HT-T1D is evaluated in therapeutic in vivo studies in diabetic NOD mice. The HT-T1D characterized, such as described above, is tested in a therapeutic T1D model in NOD mice. The NOD mouse strain is selected that spontaneously develops T1D as it reproduces many features of the clinical disease in humans, offering the possibility of testing the HT-T1D in vivo. At the onset of diabetes, NOD mice are assigned to a HT-T1D arm or several control arms. Clinical parameters indicative of T1D progression (glycemia, weight loss, mortality) are monitored over time as an indication of treatment effectiveness.

Beginning at 9 weeks of age, a total of 246 NOD/ShiltJ female mice are monitored for enrollment into the HT-T1D therapeutic study. Blood glucose is measured twice weekly and any mice with blood glucose (BG) levels between 200-350 mg/dL are retested the next day. If BG levels in that range are confirmed on two consecutive days, mice are enrolled into the study immediately, assigned to study groups on a rolling basis until each group contains 14 mice. Mice not used in the study are euthanized. Mice enrolled in the study receive split dose administration of test article (dose to be determined as described above) via sub-cutaneous injection on days 0/1, 14/15, 28/29 and 42/43. The 8 treatment groups include: untreated (PBS); rHSA; Tregitope peptides (SEQ ID NOS: 1 and 28); rHSA-Tregitope (“HT”; Tregitope peptides SEQ ID NOS: 1 and 28); rHSA-PPI (Tregitope peptides SEQ ID NOS: 1 and 28; PPI peptide SEQ ID NO: 56); rHSA-scrambled Tregitope-PPI (Tregitope peptides scrambled from SEQ ID NOS: 1 and 28; PPI peptide SEQ ID NO: 56); and rHSA-Tregitope-PPI (“HT-T1D”; Tregitope peptides SEQ ID NOS: 1 and 28; PPI peptide SEQ ID NO: 56).

Clinical observations and body weight measurements are performed on a weekly basis. For injections that occur after study Day 0, cage side observations are carried out at 1, 2 and 4 hours post injection to identify any signs of toxicity due to the treatments. Blood glucose and body weight are measured twice weekly. Mice that appear to be moribund, evidenced by hunched fur, ruffled fur, or have weight loss exceeding 20%, are euthanized. On the euthanized animals, blood is collected by cardiac puncture and PBMC separated. Cells are labeled with antibodies to phenotypic and activation status markers according to SOP (CD3, CD4, CD8, CD62L, CD45RA, CD25, CD127, FoxP3, IFNγ, CD69, IL-17 and IL-21), fixed and analyzed by flow cytometry to determine the status of CD8 and CD4 memory, effector and regulatory T cells. Levels of Peptide C are measured in serum by ELISA. Pancreas is collected and fixed for later histological examination of lymphocytic infiltration and Treg presence. The study continues for 60 days post dosing initiation. At the end of the study, the remaining animals are sacrificed, and tissue samples are collected for pathology.

In the therapeutic study, without treatment 100% of enrolled NOD mice develop diabetes. Treatment with HT-T1D at 14 mice per group, in aspects, there is expected to be a statistical 76.5% power to observe a 50% difference (from 60% to 30% penetrance) in NOD mouse model of therapeutic study. BG measurements across all available mice are compiled for a time-series analysis. Mice that reach a study endpoint that is not defined by BG (moribund) are assigned the maximum BG level (600). Differences between groups at each time point and differences in the temporal change in BG measurements are evaluated using the (i) t-test, (ii) Mann-Whitney U test and (iii) nonparametric difference test using 1,000 permutations. Comparisons of the absolute BG levels between two groups at each time point is considered statistically significant at a p-value<0.05. Treatment with HT-T1D is expected to delay onset or reverse diabetes development in NOD mice compared to PBS- or rHSA-treated control mice.

### Example 13. Evaluation of Biodistribution, Pharmacokinetics, and Immunotoxicity of HT-T1D

Immunotoxicity studies are performed to confirm the antigen-specificity and targeted immunomodulatory effect of the HT-T1D. hFCRn/alb−/−/scid trans-genic mice are used to assess Pharmacokinetic (PK) of HT-T1D.

The biodistribution is evaluated of rHSA-T1D labeled with IR or NIR dye after different routes of administration (subcutaneously (SQ), intravenously (IV), intraperitoneally (IP), or intramuscularly (IM)) into C57BL/6 mice. Mice are imaged with the LiCor Odyssey CLx Infrared Imaging System at different time points post-injection. There are 6 mice per group for different routes of injection. Labelled HT-T1D drug is injected into mice by SC, IP, IV and IM and are analyzed for distribution at time 0, 0.5 h, 1 h, 3 h, 8 h, 24 h and 48 h. HT-T1D are distributed mainly to lymph node by subcutaneous administration whereas it is distributed to liver by IP, IV and IM injections. SC administration is optimal.

Pharmacokinetic (PK) studies using human transgenic mice are performed to define the impact of human FcRn binding on HT-T1D plasma levels in anticipation of performing a human trial. hFCRn/alb−/−/scid (JAX cat. No. 031644) have a knockout allele of the FcRn α-chain and express a human FcRn α-chain transgene under control of the human FcRn promoter, are deficient for albumin, and are immunodeficient. These mice are a suitable model to test the binding of the HT-T1D conjugate to the FcRn without competition of endogenous mouse serum albumin. The immunodeficiency inhibits mice from producing any anti-drug antibodies, as these mice are not tolerant to mouse and human serum albumin.

8-10 week old mice are assigned to two groups of 15 male and 15 female mice. Each group will receive either a low (25 μg) or high dose (100 μg) of the HT-T1D SC in PBS in the inguinal fold. On each of days 1, 4, 10, 14, and 21, 0.4 mL whole blood is collected into EDTA and 0.4 mL whole blood is collected into citrate anticoagulant (a total of 0.8 mL whole blood via cardiac puncture) from 3 males and 3 females per group. Collected blood samples are centrifuged, plasma harvested and stored at −70° C. to determine evolution of HT-T1D levels. This ELISA is performed with an antibody raised to the rHSA. The HT-T1D is expected to have a prolonged half-life in the serum of hFCRn/alb−/−/scid mice, as measured after SQ injection. These mice are the best model for rHSA half-life in humans.

Potential deleterious immunomodulatory effects of the Tregitope induced adaptive tolerance on the systemic immune response to other antigens (immuno-toxicity) is evaluated by measuring the effect of HT on the T cell and antibody response to influenza vaccination. To show that the effect of HT on co-administered antigen in an in vivo mouse model, an OVA immunogenicity assay is developed in C57BL/6 mice in which the response to immunization with OVA antigen is down modulated by co-injection with HT. To address whether HT-T1D immunotherapy might decrease the immune response to other unrelated antigens, a test is done on its effect on the well characterized mouse memory T and B cell response to Fluzone influenza vaccine in C57BL/6 female mice in a three arm study: Arm A: adjuvant (Montanide); Arm B: TFP in adjuvant (starting Day 21); Arm C: OVA in adjuvant (starting Day 21); Arm D: OVA+TFP in adjuvant (starting Day 21); Arm E: Fluzone in adjuvant (immunization Day 1 and 14); Arm F: Fluzone+TFP in adjuvant (immunization Day 1 and 14); Arm G: Fluzone in adjuvant (immunization Day 1 and 14), TFP in adjuvant (immunization Day 21, opposite flanks); Arm H: Fluzone+ in adjuvant (immunization Day 1 and 14), OVA in adjuvant (immunization Day 21, opposite flanks); Arm I: Fluzone in adjuvant (immunization Day 1 and 14), OVA+TFP in adjuvant (immunization Day 21, opposite flanks).

Fluzone (5 μg pediatric) is injected S.C. with adjuvant in the inguinal fold at Days 1 and 14 (flu vaccination). At Day 21, OVA (50 μg) in adjuvant is injected with or without HT in the corresponding Arms. At Day 28, animals are sacrificed, and spleen and serum are collected. Antibodies to OVA and influenza (Fluzone) are determined by ELISA, and T cell response to OVA and flu HA are evaluated as in Example 8. HT-T1D co-administered with OVA is expected to specifically down-modulate the response to this Ag while having no effect on the memory immune response to Fluzone. Further, the HT-T1D conjugate are expected to exhibit improved stability, extended half-life, and improved immunospecificity for the HT conjugate when co-administered with antigen.

### Example 14. Evaluation of HT-T1D Conjugate in Standard Toxicity and Safety Studies

Standard toxicity and safety studies are performed, including Genetic Toxicology, Single Dose and Repeat Dose Toxicity studies and standard Safety studies. These studies are performed using the HT-T1D product to inform additional pre-clinical evaluation in preparation for human Phase I Clinical Trials.

For a pilot study, hFcRn/alb−/−/scid mice are assigned to five groups of 2 males and 2 females. Dosing is performed by a single administration of the HT-T1D S.C.; each group receiving an incremental dose based on the reaction of the previous group following 1 hr. of observation. Dosing is followed by a 5-day observation period.

For an acute toxicity study, hFcRn/alb−/−/scid mice are assigned to five groups (2 control+3 treated) of 6 males and 6 females. Treatment consists of a single administration S.C. in PBS followed by 14 days of observation. Clinical signs are monitored daily, body weights weekly. Hematology, coagulation, and blood chemistry are performed on all animals upon sacrifice. Full tissue is collected and cryopreserved for each animal at end of study or at any unexpected death or abnormality.

For a regulatory toxicology study, hFcRn/alb−/−/scid mice are assigned of five groups of 6 males and 6 females (2 control+3 treated). Treatment is by S.C. administration in PBS once weekly for two consecutive weeks (Days 1 and 8). Clinical signs are monitored daily. Body weight will be assessed at randomization, pre-treatment and at termination. Macroscopic examination of all mice is on Day 15. After euthanasia, selected organ weights (pancreas, liver, lungs, heart, kidneys) are recorded for all mice.

For a 28-day toxicity study, hFcRn/alb−/−/scid mice are assigned to four groups of 9 males and 9 females and are treated with HT-T1D by S.C. administration in PBS once weekly for 4 weeks (Days 1, 8, 15, and 21). Main study animals (6 males and 6 females per group) are observed for a further 7 days out to 28 days. Recovery phase animals (3 males and 3 females per group) are expected to survive to Day 49. Clinical signs are assessed daily. Body weight is measured pre-treatment, then weekly during treatment and at termination. Food consumption is assessed weekly. Blood samples are centrifuged and plasma/serum harvested and stored at −70° C. Toxicokinetic animals are euthanized without further investigation after the last bleed. Hematology, coagulation, blood chemistry and urinalysis of all main and recovery phase mice is performed at termination. Bone marrow, pancreas, liver, lungs, heart, and kidneys of all main study mice (control and high dose) are collected, weighed and are preserved in fixative for histological examination.

There is expected to be minimal or no toxicity of HT-T1D at the highest dose that significantly prevents diabetes in the POC study of therapeutic T1D. Neither Tregitope nor PPI are be expected to be toxic in mice nor in humans, as they are peptide sequences that are present in endogenous proteins. The safety and absence of acute or sustained toxicity of HT-T1D therapeutic will be demonstrated.

### EQUIVALENTS

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

1. A Tregitope-blood component conjugate comprising: a modified polypeptide, said modified polypeptide having a reactive moiety attached thereto and said modified polypeptide comprising one or more regulatory T cell epitopes or
wherein said modified polypeptide is further linked to a blood component.

2-3. (canceled)

4. The Tregitope-blood component conjugate of claim 1, wherein said one or more regulatory T cell epitopes comprises of one or more amino acid sequences selected from the group consisting of SEQ ID NOS: 1-55, and/or fragments and variants thereof, and optionally 1 to 12 additional amino acids distributed in any ratio on the N terminus and/or C-terminus of the polypeptide of SEQ ID NOS. 1-55.

5. The Tregitope-blood component conjugate of claim 1, wherein said one or more regulatory T cell epitopes comprises SEQ ID NO: 1.

6. The Tregitope-blood component conjugate of claim 1, wherein said one or more regulatory T cell epitopes comprises SEQ ID NO: 28.

7. The Tregitope-blood component conjugate of claim 1, wherein said blood component is albumin.

8. The Tregitope-blood component conjugate of claim 1, wherein said blood component is human serum albumin.

9. The Tregitope-blood component conjugate of claim 1, wherein said modified polypeptide further comprises a T1Dgen peptide.

10. The Tregitope-blood component conjugate of claim 9, wherein the T1Dgen peptide comprise one or more amino sequences selected from the group consisting of SEQ ID NOS: 56-63.

11. The Tregitope-blood component conjugate of claim 1, wherein the reactive moiety is attached to the amino terminal amino acid of the modified polypeptide.

12. The Tregitope-blood component conjugate of claim 1, wherein the reactive moiety is attached to the carboxy terminal amino acid of the modified polypeptide.

13. The Tregitope-blood component conjugate of claim 1, wherein the reactive moiety is attached to an amino acid positioned between the amino terminal amino acid and the carboxy terminal amino acid of the modified polypeptide.

14. The Tregitope-blood component conjugate of claim 1, wherein the reactive moiety is a succinimidyl or maleimido group.

15. The Tregitope-blood component conjugate of claim 1, wherein the reactive moiety is a 3-maleimidoproprionic acid moiety.

16. The Tregitope-blood component conjugate of claim 1, wherein a conjugation between the blood component and the modified polypeptide is a maleimide linkage.

17-29. (canceled)

30. A method for suppressing an autoimmune response characteristic of T1D in a subject in need thereof, the method comprising administering to the subject a Tregitope-blood component conjugate of claim 1.

31. (canceled)

32. The method according to claim 30, wherein the administration shifts one or more antigen presenting cells, dendritic cells, or T cells to a regulatory phenotype.

33. (canceled)

34. The method according to claim 30, wherein the regulatory phenotype is characterized by a decrease in CD11c and HLA-DR expression in the dendritic cells or other antigen presenting cells.

35. (canceled)

36. The method according to claim 30, wherein the administration of the one or more regulatory T-cell epitopes activates CD4⁺/CD25⁺/FoxP3⁺ regulatory T-cells.

37. The method according to claim 30, wherein the administration suppresses an immune response selected from the group consisting of an innate immune response, an adaptive immune response, an effector T cell response, a memory T cell response, a helper T cell response, a B cell response, a ηκT cell response, or any combination thereof.

41. A pharmaceutical composition comprising a Tregitope-blood component conjugate according to claim 1 and a carrier, excipient, and/or adjuvant.

42. (canceled)
